SEQUENCE LISTING (1368 sequences)

SEQ ID NO: 1 - DNA, 51 bp, Homo sapiens
ctctaatttcagcaaactgaaaatayggtccactaaccagcagcatcagca

SEQ ID NO: 2 - DNA, 51 bp, Homo sapiens
atgtcaaatgcctgtagtgtggcaayagaatgcttcatttcagtgggataa

SEQ ID NO: 3 - DNA, 51 bp, Homo sapiens
aaatgcatttaaagactacttacacsaagggcattatcattatacccaaat

SEQ ID NO: 4 - DNA, 51 bp, Homo sapiens
catataggcaatcaggtattggaaakgaatttgcatatagatgcaaaacaa

SEQ ID NO: 5 - DNA, 51 bp, Homo sapiens
tcataacccttttataactagtaacmactcttactccttgagaatagctaa

SEQ ID NO: 6 - DNA, 51 bp, Homo sapiens
atatagcacaagcttcagttttaaartaatctgtaatatacactcaactct

SEQ ID NO: 7 - DNA, 51 bp, Homo sapiens
atttttaaaatgactttcaaagacarctaatgaacaagacaatatgtaagg

SEQ ID NO: 8 - DNA, 51 bp, Homo sapiens
ggattcaaggatatattttgtctacyggccctcatgtttgtatgtacttga

SEQ ID NO: 9 - DNA, 51 bp, Homo sapiens
ctgcatgagccaattccttgaggtasatatctttatatataaatagactgt

SEQ ID NO: 10 - DNA, 51 bp, Homo sapiens
ggttaaatagattttctaagttgaarcagtgtcttaatggcttcaatattt

SEQ ID NO: 11 - DNA, 51 bp, Homo sapiens
gtgtttgacaaatttttgcttaccawctttaatatttaagtgaggtaaaat

SEQ ID NO: 12 - DNA, 51 bp, Homo sapiens
ctgagatttagattaaaggctatgartacgccaaacagcaattatttcctt

SEQ ID NO: 13 - DNA, 51 bp, Homo sapiens
agagtctagcaagaaaggacctaccyaagtacaagggattgtcatcaaggt

SEQ ID NO: 14 - DNA, 51 bp, Homo sapiens
tcacgtagactaacctcagtacagtstaggagagaactatgcaagggtcta

SEQ ID NO: 15 - DNA, 51 bp, Homo sapiens
tatatgcaaacatatttatcagggayccatcaaagttcagcttcagctaca

SEQ ID NO: 16 - DNA, 51 bp, Homo sapiens
caaaaacaacaaacattgtccctacrcctttacatctattcacctttttac

SEQ ID NO: 17 - DNA, 51 bp, Homo sapiens
gaagagaggaataatgagacaactarggaaaccagacaagaccatcttagc

SEQ ID NO: 18 - DNA, 51 bp, Homo sapiens
gaaacaccaagaatttcagtaaataraggtagctgcggtgctaaatgctat

SEQ ID NO: 19 - DNA, 51 bp, Homo sapiens
tcaagaaaaaggcttagtttgtaaartaagctctatctgcatactggaagg

SEQ ID NO: 20 - DNA, 51 bp, Homo sapiens
gaatacttcttctctccatactctaygcatgtctgggaaaggctccaaagg

SEQ ID NO: 21 - DNA, 51 bp, Homo sapiens
ttattcaagataaaagaggaattggmaacctatcccaggcttgtttttgca

SEQ ID NO: 22 - DNA, 51 bp, Homo sapiens
aaatgtagtctagaaagtaattgtgragttttctcatgtttgaattaatgt

SEQ ID NO: 23 - DNA, 51 bp, Homo sapiens
gaattctcaaagtttggtcctgccartaagtagtacatccagatatatgca

SEQ ID NO: 24 - DNA, 51 bp, Homo sapiens
tgggactcaggctaggtcatcctcgyagtagctgtaaagttttctgaattt

SEQ ID NO: 25 - DNA, 51 bp, Homo sapiens
tataactaatacaaaatgtactttgkaacttgtcgccagatatttttttcc

SEQ ID NO: 26 - DNA, 51 bp, Homo sapiens
gcaatgtggtcatatgttcctaagawgcacaattattgaaaactttaatta

SEQ ID NO: 27 - DNA, 51 bp, Homo sapiens
cttttgagtgactttctcacttcacrctcaatgtcagtcactcacagagat

SEQ ID NO: 28 - DNA, 51 bp, Homo sapiens
caaaagcaacttgaaaatgctttgcrcccataattaaactctttatttcat

SEQ ID NO: 29 - DNA, 51 bp, Homo sapiens
agagcatctcttgctacctccattcraagagactgatgttttctgtgtaat

SEQ ID NO: 30 - DNA, 51 bp, Homo sapiens
gcaacattatatttcataaagaccayggtgtagagtaaatcaagttttccc

SEQ ID NO: 31 - DNA, 51 bp, Homo sapiens
gggcaggtatctggaaaccaggcaayatacgccttgggcatctgattcttt

SEQ ID NO: 32 - DNA, 51 bp, Homo sapiens
aaacataatgttggcttcagcatccracaggtataaattctatgctcataa

SEQ ID NO: 33 - DNA, 51 bp, Homo sapiens
gttcattgttaatcggtaagacaaarctaagaacataaataccaatgatga

SEQ ID NO: 34 - DNA, 51 bp, Homo sapiens
cactgcaatcttaaaggcaacgaacycgctttttagtattttgaaaggttg

SEQ ID NO: 35 - DNA, 51 bp, Homo sapiens
caggaaaatgagacacttcctctgaycttagactaggctgggtttccagct

SEQ ID NO: 36 - DNA, 51 bp, Homo sapiens
cagaatatctaaattgaaacaatggraaactcaattaaaaatatgtttagc

SEQ ID NO: 37 - DNA, 51 bp, Homo sapiens
tgagctgaggagtgtaatcaagtcamccttttacactggagatccaaaaat

SEQ ID NO: 38 - DNA, 51 bp, Homo sapiens
gatatcaggcatctccataattacargtggctatagaaatcaggaactgcc

SEQ ID NO: 39 - DNA, 51 bp, Homo sapiens
ctaacctttacagtacactttcccartggaatatacagtctgtgtaccaag

SEQ ID NO: 40 - DNA, 51 bp, Homo sapiens
tgcctagctgctaggtacctcaagaytaagatctctccttagtagttatag

SEQ ID NO: 41 - DNA, 51 bp, Homo sapiens
ctaccaatgtattagtccactttccyaaatgtgtaagtgaacataattttg

SEQ ID NO: 42 - DNA, 51 bp, Homo sapiens
cctgtctgtccagctcagcactctayctaagaaatcctagaagctgggaat

SEQ ID NO: 43 - DNA, 51 bp, Homo sapiens
tggcccccttcttgttggcctaacayagagctttggaattgcttggttgcc

SEQ ID NO: 44 - DNA, 51 bp, Homo sapiens
ccaaatgcctccatttcctagaataraaggtcacattgttcattacactgg

SEQ ID NO: 45 - DNA, 51 bp, Homo sapiens
agtgtccttctatgctctggcagaayctctggctcaggcacctgtgctgta

SEQ ID NO: 46 - DNA, 51 bp, Homo sapiens
tgtttaagactttgatgtgcctaaarttaatctagatgaaatgaatgaggg

SEQ ID NO: 47 - DNA, 51 bp, Homo sapiens
gcttgggtccagattcagataccccratggttctgactgggttctggttcc

SEQ ID NO: 48 - DNA, 51 bp, Homo sapiens
atgagcttagaaacactggaaccacygatatacaaaagtatttttatcata

SEQ ID NO: 49 - DNA, 51 bp, Homo sapiens
ttttcctcttaagtagtaaaaaggtsaatcattggaaagatctcggagaga

SEQ ID NO: 50 - DNA, 51 bp, Homo sapiens
ttaaacgtctattggttaaatgatcrgagaatcagagaggagaaagcagtc

SEQ ID NO: 51 - DNA, 51 bp, Homo sapiens
atgcagaaatgtcaggaacgctcaaygcggggacaatctacaaaacaaccg

SEQ ID NO: 52 - DNA, 51 bp, Homo sapiens
ggatacagtctgtatttctttaaaaytgtaagcagtctcctaaaatccttt

SEQ ID NO: 53 - DNA, 51 bp, Homo sapiens
acactttgaaatgataagatgctaamgaacgtcaccctgagctgcagtgtt

SEQ ID NO: 54 - DNA, 51 bp, Homo sapiens
acttgtgggttctgtgtcagtggaaygtttcagtactttttggcctggctc

SEQ ID NO: 55 - DNA, 51 bp, Homo sapiens
aggtcctcatagttgatggtttaccyactgatttgaacaaacgaactgtta

SEQ ID NO: 56 - DNA, 51 bp, Homo sapiens
agctaataaaagtaaattctttcccracttgttcctacttcaaaagaaatg

SEQ ID NO: 57 - DNA, 51 bp, Homo sapiens
ctgcaatgacagtcaacatagaaaasaagggtacacttggctgtaaaaagc

SEQ ID NO: 58 - DNA, 51 bp, Homo sapiens
agcactgcagtctgttattggaccayaaaatatagaggtaagtccttccct

SEQ ID NO: 59 - DNA, 51 bp, Homo sapiens
atccaaaaaatatggtctctttaacrccattacaggggtggctttacagat

SEQ ID NO: 60 - DNA, 51 bp, Homo sapiens
ggtgcctgtagcctggatgatcagcrcattccgaggtttagggatggggcc

SEQ ID NO: 61 - DNA, 51 bp, Homo sapiens
aaatttaatttctgtagtagttagcyatgtctcatcatattatttaaaata

SEQ ID NO: 62 - DNA, 51 bp, Homo sapiens
taaaaacagaatatttggggatgaarcattattcttttcagttgtcatttt

SEQ ID NO: 63 - DNA, 51 bp, Homo sapiens
tcttagacaaggcattatgcaaccaygggataagtgcggttctagaagcca

SEQ ID NO: 64 - DNA, 51 bp, Homo sapiens
acataagaaaaattatagaaaagagmaccaatgcataccgacaaaagtgag

SEQ ID NO: 65 - DNA, 51 bp, Homo sapiens
aactactattatttaaacaacatgaygaatgagaatgcatatataaatatt

SEQ ID NO: 66 - DNA, 51 bp, Homo sapiens
tttctcttccctgatgccataactaycttccaaaatgtaaacataatgatg

SEQ ID NO: 67 - DNA, 51 bp, Homo sapiens
tgcttctcaaaagggatctaaggcaycttaaaataaaattcatgtatataa

SEQ ID NO: 68 - DNA, 51 bp, Homo sapiens
aggctgcttccatagctagtctagcygaaccatttccgagctacaaggcag

SEQ ID NO: 69 - DNA, 51 bp, Homo sapiens
cagagaagagataaacagaattcagsaacagctaactccaagtcagataat

SEQ ID NO: 70 - DNA, 51 bp, Homo sapiens
taagactcttgataacatctaaacargtatttttcaagtctagactgtttt

SEQ ID NO: 71 - DNA, 51 bp, Homo sapiens
gaaaactagtagaaatatcacttcayggatggcttttgctgggtgttgaag

SEQ ID NO: 72 - DNA, 51 bp, Homo sapiens
ggattcaatcaggtgggaacctgaamgtctgtgacatctgaagtgtgtagt

SEQ ID NO: 73 - DNA, 51 bp, Homo sapiens
agacaggagcacgtaagagggagatyacaacacagtgtgagaagtacaaag

SEQ ID NO: 74 - DNA, 51 bp, Homo sapiens
aaaaagaaaagctaattcaaaaccayggatgtaattactaagattaaaaga

SEQ ID NO: 75 - DNA, 51 bp, Homo sapiens
aattcagtgaaaacatttgggtgaargttcctatgccacaaagttaaatac

SEQ ID NO: 76 - DNA, 51 bp, Homo sapiens
tatcaaagtttacaaagtgctattcragggagattaacctgacagtagtat

SEQ ID NO: 77 - DNA, 51 bp, Homo sapiens
gagcagtttactagtttctggataamccgggcctttattcttccctcccta

SEQ ID NO: 78 - DNA, 51 bp, Homo sapiens
actcaggcagaatcctagaaaggaastgaaatttgatctgggcaatttttc

SEQ ID NO: 79 - DNA, 51 bp, Homo sapiens
aaaaaattccttaagggtgtataacrgcatcctaaagagagcttagatgga

SEQ ID NO: 80 - DNA, 51 bp, Homo sapiens
acgggcaagcctccccatgaagagaygatccaggtgcgacacaggaggtgc

SEQ ID NO: 81 - DNA, 51 bp, Homo sapiens
gcaaggtgatccatgcttgcaaccasgacaaaacaagcatgcagtaggcac

SEQ ID NO: 82 - DNA, 51 bp, Homo sapiens
gggaataataaaataacagatttctracacttaggcatttcacaacttatt

SEQ ID NO: 83 - DNA, 51 bp, Homo sapiens
aagcagttatctctccttactcccartggaaacccttgactccaagttgac

SEQ ID NO: 84 - DNA, 51 bp, Homo sapiens
aaaaaaccacatagtgaagttgacastcaggaactgctgaaattttatcaa

SEQ ID NO: 85 - DNA, 51 bp, Homo sapiens
tgatcacgctagatgctcgcatggtsaaacggcaagacgcagcttcttcct

SEQ ID NO: 86 - DNA, 51 bp, Homo sapiens
gcgtctttcccaagcactccacggarcgtccttaggcacctccctctctcg

SEQ ID NO: 87 - DNA, 51 bp, Homo sapiens
ctcagagggtttagcaaataaagaaygatgtaggtcacaaagcaaaggaaa

SEQ ID NO: 88 - DNA, 51 bp, Homo sapiens
aagtttccccacaacatatttgcaaytctgtgatggagtaagtcttgaaaa

SEQ ID NO: 89 - DNA, 51 bp, Homo sapiens
gccatcatgaactttaatctgattayggtcctaaaactacttcttcacgaa

SEQ ID NO: 90 - DNA, 51 bp, Homo sapiens
ctagcctgctaaacttgaaaactaaygagaagtataccaaaaattgcttcc

SEQ ID NO: 91 - DNA, 51 bp, Homo sapiens
taaagagaaatgaagaaccacagtakggagtacttttctgcattgggtagg

SEQ ID NO: 92 - DNA, 51 bp, Homo sapiens
aatatgaaaaatattttacagagatyacatttgacgcagctgcttcctgaa

SEQ ID NO: 93 - DNA, 51 bp, Homo sapiens
aacattaacacagaagttggggaacsgaccgagaagaggttgatctagaat

SEQ ID NO: 94 - DNA, 51 bp, Homo sapiens
tctttgctcacattaaagtcccttargtgagtataattcttctctgatgac

SEQ ID NO: 95 - DNA, 51 bp, Homo sapiens
tcaggggcacgcacagtaggctgaaytgcaacgagagcacttggcaatccg

SEQ ID NO: 96 - DNA, 51 bp, Homo sapiens
tcatcaaagaaaacagcaacacaaaygagacacaatacctaccactcatgg

SEQ ID NO: 97 - DNA, 51 bp, Homo sapiens
ttctagacattcagacaacattttcrctctcacatgaaatttgtaaacaat

SEQ ID NO: 98 - DNA, 51 bp, Homo sapiens
tggatgatgttagtttcgaaacctayagcccataatctggactcagctaaa

SEQ ID NO: 99 - DNA, 51 bp, Homo sapiens
tgtgaactcagctagcgtcaatcaartaaataacctgttaacaatgtcctt

SEQ ID NO: 100 - DNA, 51 bp, Homo sapiens
cagtatgtttactatgcatatatttyaaatagttgtaatcctaacatagat

SEQ ID NO: 101 - DNA, 51 bp, Homo sapiens
tccattgttccaatactaactgagckctatcaacttcaaatactgctgctg

SEQ ID NO: 102 - DNA, 51 bp, Homo sapiens
aggaatcctaggtagctttcatagayggagactccctgcacaccccaaatg

SEQ ID NO: 103 - DNA, 51 bp, Homo sapiens
aacacttcggcctagataacagatgracctttccactatgccagcacaggt

SEQ ID NO: 104 - DNA, 51 bp, Homo sapiens
atgcatcccagaactcacttctatayctaatactacccgtttcttgataca

SEQ ID NO: 105 - DNA, 51 bp, Homo sapiens
ctgaagaattacaagtaaggaacgaygattattagagattagaaagcattg

SEQ ID NO: 106 - DNA, 51 bp, Homo sapiens
caatcatcttagcttggttcattagyagagcatttttagtaactgtaacac

SEQ ID NO: 107 - DNA, 51 bp, Homo sapiens
aaatgtttcaggaaataatttctatragttgattctgaaaataaatcagta

SEQ ID NO: 108 - DNA, 51 bp, Homo sapiens
aggcaacagaagcaaaagagtctcaygcgttgattatacaaatacagccca

SEQ ID NO: 109 - DNA, 51 bp, Homo sapiens
ttcttgccattttcaccagatggacyctatcgtaacaatcaattttttaga

SEQ ID NO: 110 - DNA, 51 bp, Homo sapiens
accgcattaatttaggaaaatgtaayctgtaagtccagttgacttaattgc

SEQ ID NO: 111 - DNA, 51 bp, Homo sapiens
tctcaatgacctgaaaaatgaccaamtgcattaaaagttcttggggtgcat

SEQ ID NO: 112 - DNA, 51 bp, Homo sapiens
gaataccaaggccttgatgtgtccartatttactcaggagcttcactacag

SEQ ID NO: 113 - DNA, 51 bp, Homo sapiens
ttattttctgtcagattttgggtacygaataagccaagagggcatactgct

SEQ ID NO: 114 - DNA, 51 bp, Homo sapiens
ggcataccagatcctacctcatggarccctaccgtaactctatattcttac

SEQ ID NO: 115 - DNA, 51 bp, Homo sapiens
ggaagagagggcaagggttagagaaragcttgggccgcagcattgcctctg

SEQ ID NO: 116 - DNA, 51 bp, Homo sapiens
gctatgttagatactccagttcattsaaagtgatatcaccctaacattctc

SEQ ID NO: 117 - DNA, 51 bp, Homo sapiens
ttcctaaggaaacccactgccacgakatctgttttccaggccttgaatgct

SEQ ID NO: 118 - DNA, 51 bp, Homo sapiens
aaatactgtaagggagaaagcaagaygcactattttgaaaatgaaaaatag

SEQ ID NO: 119 - DNA, 51 bp, Homo sapiens
aataactattagggcaattggtaaayaatgtatcaaaaaatgaaataaatg

SEQ ID NO: 120 - DNA, 51 bp, Homo sapiens
cttgctttaaaactttctcttcttayaggttgtgaatatttccatgcaaat

SEQ ID NO: 121 - DNA, 51 bp, Homo sapiens
tagccatatgtttttggctttcatartatactgtaggctgatagctggctt

SEQ ID NO: 122 - DNA, 51 bp, Homo sapiens
taatatacattcactcaagtgttaastgatacaaacaaattaatgagtcaa

SEQ ID NO: 123 - DNA, 51 bp, Homo sapiens
tcagatttgaataaatgaggtatttsaaaaggtattactctgtgggaagtt

SEQ ID NO: 124 - DNA, 51 bp, Homo sapiens
ttacccttcctttgtagatgaataawttaaaatatccagtaaattgatttt

SEQ ID NO: 125 - DNA, 51 bp, Homo sapiens
ggctggctcacctgtattattctcayagatttccaataatatgaaaaaagt

SEQ ID NO: 126 - DNA, 51 bp, Homo sapiens
ctgtaccagaagataaatgttgaaaytaagcaagaactgtattttactcaa

SEQ ID NO: 127 - DNA, 51 bp, Homo sapiens
aaacaaatgagtacataattcacaaytggagcttaggaaaaattcccagta

SEQ ID NO: 128 - DNA, 51 bp, Homo sapiens
agaccatgatgtaaaatgatatgaaygaaggagaaagaacaaattttgata

SEQ ID NO: 129 - DNA, 51 bp, Homo sapiens
gactatgcatacttgcttagaatccmaaaggcaaagagcagatgggttgaa

SEQ ID NO: 130 - DNA, 51 bp, Homo sapiens
agttagctttgccccagtagtggccygaaaacacctacctaatgcctgttg

SEQ ID NO: 131 - DNA, 51 bp, Homo sapiens
tgatgccgagtttttgaaattttgaygtatctgggcattaattgaaaaagt

SEQ ID NO: 132 - DNA, 51 bp, Homo sapiens
aattaatcagctctgatatcaatcawccagttacacattaatatgtaattc

SEQ ID NO: 133 - DNA, 51 bp, Homo sapiens
agagggttcatttatactattcaaaycatacatatatacataaaaatgaaa

SEQ ID NO: 134 - DNA, 51 bp, Homo sapiens
ttgcaccacaaacaccttgagatgarcttataataaaggttgacttgtttc

SEQ ID NO: 135 - DNA, 51 bp, Homo sapiens
gaacagcattagaagagtcaggtaamcaataaaagtaagtacaaaatagaa

SEQ ID NO: 136 - DNA, 51 bp, Homo sapiens
ttaatacataagacatgcatgcctaygatctcttatttcaggacaaccaga

SEQ ID NO: 137 - DNA, 51 bp, Homo sapiens
tggcagatacagtcacttaccagtayattgagttgagtttatagttctcag

SEQ ID NO: 138 - DNA, 51 bp, Homo sapiens
cgttccaccttagccagatctgctcrcacaggaaagaaaaccgaaggggct

SEQ ID NO: 139 - DNA, 51 bp, Homo sapiens
aaaagagagtttggatcactagaaamcttccaagaagaaaagaatcctgac

SEQ ID NO: 140 - DNA, 51 bp, Homo sapiens
tttttgtatttttcagtagttttatsagtagcagtcttttgtaatttttga

SEQ ID NO: 141 - DNA, 51 bp, Homo sapiens
aagtctcactgtggcaatctccttargattcaaacccagagttaaggctgg

SEQ ID NO: 142 - DNA, 51 bp, Homo sapiens
aacataggcccacctacacacgttaygaggtgcttaagtgaaagaactgtg

SEQ ID NO: 143 - DNA, 51 bp, Homo sapiens
ttggaaagtagaacagttatatgtakggaaagttgcatgtagtgaagtggt

SEQ ID NO: 144 - DNA, 51 bp, Homo sapiens
tcagaatcccctctgtagacctttgraatgcaatttcctccactctactaa

SEQ ID NO: 145 - DNA, 51 bp, Homo sapiens
tcctggaatgaacggctaaatgaatmataaattagattaataaaatactca

SEQ ID NO: 146 - DNA, 51 bp, Homo sapiens
tctttttattacaccggcatctaacmaaatgaatttgatagaatatgaatt

SEQ ID NO: 147 - DNA, 51 bp, Homo sapiens
aaaagcgtattgcttaaatttgaccragcttttcctaaatcctatcagttt

SEQ ID NO: 148 - DNA, 51 bp, Homo sapiens
gttttgaagactcaagaggttgatayattttggaagggatcacaattgact

SEQ ID NO: 149 - DNA, 51 bp, Homo sapiens
ttcaactgacaggctttaacctttakactgctgaagttataaaatgtatag

SEQ ID NO: 150 - DNA, 51 bp, Homo sapiens
tctttattaagaataactaatgtccragaacacattttgatgcatattgtg

SEQ ID NO: 151 - DNA, 51 bp, Homo sapiens
tagatcacaagcaaattagtctgacrccatgaactctccccctcactccac

SEQ ID NO: 152 - DNA, 51 bp, Homo sapiens
tgaaattgttgcttatgatgatgtargagagattgcctataaatgggaagg

SEQ ID NO: 153 - DNA, 51 bp, Homo sapiens
acgtataaaaccgtggatactgttaycatgtgtgtaaaagaaaaattgtaa

SEQ ID NO: 154 - DNA, 51 bp, Homo sapiens
cctgttcctagtttcacaattacccraaaaataaatagattgtattagtct

SEQ ID NO: 155 - DNA, 51 bp, Homo sapiens
cccttgggccaaatatgaaaagcctmacattgcacatggagcccttgggta

SEQ ID NO: 156 - DNA, 51 bp, Homo sapiens
cgaggagaaggcaagttcatcctcamgtttcacatttcacaccaatctaaa

SEQ ID NO: 157 - DNA, 51 bp, Homo sapiens
atttatcacaattccaatgaaaataycaccaaactttcctatggagccaaa

SEQ ID NO: 158 - DNA, 51 bp, Homo sapiens
agcttgcatatttgctgaagtactcycgtatcaaactagaacagattcata

SEQ ID NO: 159 - DNA, 51 bp, Homo sapiens
gtttttgttatagctaacagagaaaygctagccaaggattttattgctggc

SEQ ID NO: 160 - DNA, 51 bp, Homo sapiens
aaaaaataattcttagctctttggawgtaattttaacaggtacattctagc

SEQ ID NO: 161 - DNA, 51 bp, Homo sapiens
tacaatggtagaaatcttagggtaartgttctgaattaactgacccatgag

SEQ ID NO: 162 - DNA, 51 bp, Homo sapiens
acatgttctattgcctcatgcagtastacattttagagcgcaagaatgttg

SEQ ID NO: 163 - DNA, 51 bp, Homo sapiens
cctttcctcgggtcatcttttcaaartcatatgccttcccctagtagtttc

SEQ ID NO: 164 - DNA, 51 bp, Homo sapiens
ttgtaggatattaatcagctccttgmagtaatgtatatcttgtttgatgag

SEQ ID NO: 165 - DNA, 51 bp, Homo sapiens
gtccatgattctctgatttggcaaaragaacttggtcactgcttttgctgc

SEQ ID NO: 166 - DNA, 51 bp, Homo sapiens
aggaagactagtcacagaagtatcaraagaagttttgcgggccgtgcacgg

SEQ ID NO: 167 - DNA, 51 bp, Homo sapiens
aatgaatttttctctatgccaaatawcttgggaaattgattctcctacttt

SEQ ID NO: 168 - DNA, 51 bp, Homo sapiens
atgggatttggatgctaatatctaaracgcccttctaagaggtccttctag

SEQ ID NO: 169 - DNA, 51 bp, Homo sapiens
cctaagtctatcttgcagtactgatyaaaagcaattttcaaatccaaaaca

SEQ ID NO: 170 - DNA, 51 bp, Homo sapiens
attattattcagttatcttaaaaggwcctaagtctatcttgcagtactgat

SEQ ID NO: 171 - DNA, 51 bp, Homo sapiens
catttctcagctttgaaggcaaggargttctagaccaagattcccctctgg

SEQ ID NO: 172 - DNA, 51 bp, Homo sapiens
atgcagtcaaagcaattgatgaaaarctccttccaatgtgtataacaaaac

SEQ ID NO: 173 - DNA, 51 bp, Homo sapiens
ggcaagttgtagtcttctctgttcaytctatcagttaaatttccctccatt

SEQ ID NO: 174 - DNA, 51 bp, Homo sapiens
taaatctgatctataaatctcatacraatctggaaattagacccagttttt

SEQ ID NO: 175 - DNA, 51 bp, Homo sapiens
acaaaagcttcttagattgtcattayagactgtaaccaaatattgttgtgt

SEQ ID NO: 176 - DNA, 51 bp, Homo sapiens
atcatgatggccttgtgtccaatgarccatttaaggggacactgtttagaa

SEQ ID NO: 177 - DNA, 51 bp, Homo sapiens
ttgctttatcaccactatcttctcckacactgatgcctttcctggggccag

SEQ ID NO: 178 - DNA, 51 bp, Homo sapiens
cattggtgtgaaaatattagtagcamaaaacttcaaaagtctagttttcat

SEQ ID NO: 179 - DNA, 51 bp, Homo sapiens
catagaagcaaaggcaaaaactgacyacagtactaaacagtcagaaacaat

SEQ ID NO: 180 - DNA, 51 bp, Homo sapiens
ttctctcggggaaagggccttggtgsaattaacagcttttaagccagagaa

SEQ ID NO: 181 - DNA, 51 bp, Homo sapiens
taaaaattacctggaggatgattacractgaactgcaggatgattaagaat

SEQ ID NO: 182 - DNA, 51 bp, Homo sapiens
caaagagatgtgcatgagtgctttaracacaagagcaagttagcgaagaga

SEQ ID NO: 183 - DNA, 51 bp, Homo sapiens
cttatccagagggcttaaccaaaccragagaatgtgtgtgtgtatctacct

SEQ ID NO: 184 - DNA, 51 bp, Homo sapiens
cagccaccacttatgaagtcagtaawtaatattcaatttgcctagagcttt

SEQ ID NO: 185 - DNA, 51 bp, Homo sapiens
ttgacataattgaataatggaatgamaaaaatcttacatggcaaattggat

SEQ ID NO: 186 - DNA, 51 bp, Homo sapiens
agcttccccaaaattttgaaggaaarttgagaaattgagaggggagattaa

SEQ ID NO: 187 - DNA, 51 bp, Homo sapiens
caccaccctattcccaggagctagamaataatttatggaatatcggctgaa

SEQ ID NO: 188 - DNA, 51 bp, Homo sapiens
cttagataggactatagtttacaaakcatctccacgtgcactgtctcatct

SEQ ID NO: 189 - DNA, 51 bp, Homo sapiens
aagtaaacacacttgctggaattgartatgttccttgattaactgcaattt

SEQ ID NO: 190 - DNA, 51 bp, Homo sapiens
acaaataaaaagtcaaattttctgcrcctttcaaaaatgatgtgtaatgat

SEQ ID NO: 191 - DNA, 51 bp, Homo sapiens
aattgggtagcccaacataatcacawgggtttttattgaatgaagaagatt

SEQ ID NO: 192 - DNA, 51 bp, Homo sapiens
catgtcactgctgggcaagttccaayaaatgtctcaagaagattagatcta

SEQ ID NO: 193 - DNA, 51 bp, Homo sapiens
tttttttccccaaaaaccacgttaaragacagtccagaaatgagattgctt

SEQ ID NO: 194 - DNA, 51 bp, Homo sapiens
ttgggactctgttaaaacttcactgyagtaatgcctttctctagactataa

SEQ ID NO: 195 - DNA, 51 bp, Homo sapiens
aacaactgtcgaagctaagtaaagaratattctagtattcagtccattttt

SEQ ID NO: 196 - DNA, 51 bp, Homo sapiens
caaacttggctgtgtgttggaatcaycttatcactattgttaattttgtaa

SEQ ID NO: 197 - DNA, 51 bp, Homo sapiens
ttcacacataagaaagcataattgaygataggtaagagagggagaatatgg

SEQ ID NO: 198 - DNA, 51 bp, Homo sapiens
ttccctatttaactgtaagataattwaaaaaaaacaaatctagatagagta

SEQ ID NO: 199 - DNA, 51 bp, Homo sapiens
cttctcataaaaaatctgtactctayggcaggggactcagatctttggtag

SEQ ID NO: 200 - DNA, 51 bp, Homo sapiens
gccagtgacctttatatgtcagttayggatttcatcatctgatctacagta

SEQ ID NO: 201 - DNA, 51 bp, Homo sapiens
gatgaattcggttaccagctgaagayagtttctttgaaataaatatcttga

SEQ ID NO: 202 - DNA, 51 bp, Homo sapiens
catgatctctacagtgtaatattgaycaactttcaaacatttgaaaagctg

SEQ ID NO: 203 - DNA, 51 bp, Homo sapiens
tcactgattccactgaattacactcwattatactgggtcagtaagcttaaa

SEQ ID NO: 204 - DNA, 51 bp, Homo sapiens
ccatgctttttaatgaaataagcacrgtcctttacttttcacatcttgact

SEQ ID NO: 205 - DNA, 51 bp, Homo sapiens
gaggatctatgctgatgttcaaatayggacaaggacatgggataaaagaaa

SEQ ID NO: 206 - DNA, 51 bp, Homo sapiens
cttaatagtttttccatttcacaatwacttattctaaagcaatactaggtc

SEQ ID NO: 207 - DNA, 51 bp, Homo sapiens
aacctcatagtgagaaatatctacaktagtgcaaaggattgtgtatgataa

SEQ ID NO: 208 - DNA, 51 bp, Homo sapiens
agtcacgaaggaatcgtaggtaacaygtaaaaggaaggagctgtaatcctt

SEQ ID NO: 209 - DNA, 51 bp, Homo sapiens
tctctgttgctgtaacactttaccascttcaagacagcaaaggaggtgtgt

SEQ ID NO: 210 - DNA, 51 bp, Homo sapiens
agtgtcttttttcccttctcaaaacyattaacccagatttccttcttattt

SEQ ID NO: 211 - DNA, 51 bp, Homo sapiens
tagaagtgtctgagatggcctagtaragtaagagaaacaaagaaaaatcct

SEQ ID NO: 212 - DNA, 51 bp, Homo sapiens
attttttaatgactaatcctcctgayatgagttcaagaagaaatagggtag

SEQ ID NO: 213 - DNA, 51 bp, Homo sapiens
gcttaccacacttttcttagtatackcattgccaaagtgggattcctgtca

SEQ ID NO: 214 - DNA, 51 bp, Homo sapiens
ttgacttattttaaagaaataccaasaatcaatgattagaaaggaaagata

SEQ ID NO: 215 - DNA, 51 bp, Homo sapiens
aatggacaacacattacagatttatyaactgcatgaccgtgaaaatatcta

SEQ ID NO: 216 - DNA, 51 bp, Homo sapiens
ctgctatgcctgtggaaattgtgacmactccttctagcctaagactaagac

SEQ ID NO: 217 - DNA, 51 bp, Homo sapiens
tttatttcattatattcctacacaayaaggacaaagaggtgtataggttga

SEQ ID NO: 218 - DNA, 51 bp, Homo sapiens
taatttaagaaaatttaacaggtgtraaaatcaggcaatgataaaaatgat

SEQ ID NO: 219 - DNA, 51 bp, Homo sapiens
gggaaataagttttcaatagcactakacttaggcttaactcacgttattaa

SEQ ID NO: 220 - DNA, 51 bp, Homo sapiens
ttacagttcatacccatgtaaatcaygtagttttctttctctggaatatat

SEQ ID NO: 221 - DNA, 51 bp, Homo sapiens
atcaccaaacacatgatcctgaataygacagagattcactgttattgataa

SEQ ID NO: 222 - DNA, 51 bp, Homo sapiens
ttcaagagcaccttctgtgagccaayttgagcctccagaatgactaaccac

SEQ ID NO: 223 - DNA, 51 bp, Homo sapiens
tgaatggctcttgcaaaccttaacaygcaaagctaatgacagattgtatca

SEQ ID NO: 224 - DNA, 51 bp, Homo sapiens
tgatgacgtggaaaaatgactctcasgagtttacgctgggatcttagttcc

SEQ ID NO: 225 - DNA, 51 bp, Homo sapiens
agagatttaactctctaagcgaccaycatttctgccaatgttttccaaaat

SEQ ID NO: 226 - DNA, 51 bp, Homo sapiens
ttttattaccaagtcctcatttcacrattatgactgacaaatgtgtggcaa

SEQ ID NO: 227 - DNA, 51 bp, Homo sapiens
agcatgtggaacttcttaacagtgtraaatatgagaggggtgggcaaatac

SEQ ID NO: 228 - DNA, 51 bp, Homo sapiens
tccaccaaatatttcagaatcagaaytcacactatcctttatattcttaga

SEQ ID NO: 229 - DNA, 51 bp, Homo sapiens
ttatttaatggacaaatacatgcackatcaggtggttacatgaatctccat

SEQ ID NO: 230 - DNA, 51 bp, Homo sapiens
ctttgacaatatgtaccaataagacraaaaggatagaatttgtagaactct

SEQ ID NO: 231 - DNA, 51 bp, Homo sapiens
tgcaccaaccctaggactcctattcrgaataaaataaggaacttcaaattt

SEQ ID NO: 232 - DNA, 51 bp, Homo sapiens
tctctcatttaccctacacagcttamaaactaatgataacgtttgaattaa

SEQ ID NO: 233 - DNA, 51 bp, Homo sapiens
aaaatgttatccaaggaatacaagaygagatttatctctcaagcaatgaca

SEQ ID NO: 234 - DNA, 51 bp, Homo sapiens
ttttatctagctctatgaaaacacaygagtagcagacacatcttaagagga

SEQ ID NO: 235 - DNA, 51 bp, Homo sapiens
gtttttggggtttccaaacacaaagycacccaaactacatgtaaatcccac

SEQ ID NO: 236 - DNA, 51 bp, Homo sapiens
ttaaaaacatgaggagctgaaaatawgtgtaactgattgcatattgtcctg

SEQ ID NO: 237 - DNA, 51 bp, Homo sapiens
ggtaattatatttactaaatatttaygttaagagttatctgtttgagctta

SEQ ID NO: 238 - DNA, 51 bp, Homo sapiens
attaccccaacttatctgaccataaragccaacatgccatcacagatggcc

SEQ ID NO: 239 - DNA, 51 bp, Homo sapiens
tggcttccttgtccttgcatagggargaacacaggcagacgtggtaggaga

SEQ ID NO: 240 - DNA, 51 bp, Homo sapiens
ctctctggctgtgtgctccactaagrctgtctacagtagaatagatgctat

SEQ ID NO: 241 - DNA, 51 bp, Homo sapiens
aggaacaaggagatgagtccagaaargtagattgtgagctttgtattctgg

SEQ ID NO: 242 - DNA, 51 bp, Homo sapiens
cattgtatagattgatattggcttawcactgaatttttaggggaaaaaaac

SEQ ID NO: 243 - DNA, 51 bp, Homo sapiens
cagcattttggcaaaaccagcaggasaagtttagaagaacatatcagtcaa

SEQ ID NO: 244 - DNA, 51 bp, Homo sapiens
aaacagcatagtagtactcatgttgraatcgcaaccagactagagatgtca

SEQ ID NO: 245 - DNA, 51 bp, Homo sapiens
tggtgaaacaagggctctacacttgsagtctgctaacaaggctgatccatg

SEQ ID NO: 246 - DNA, 51 bp, Homo sapiens
gcaaattctactggacattaagaaaycgtgtagacaaaagagatatttaag

SEQ ID NO: 247 - DNA, 51 bp, Homo sapiens
aatgaatgcataccatgtataagaakagactctggagaatttggcagcaaa

SEQ ID NO: 248 - DNA, 51 bp, Homo sapiens
aggagagttgcttaacattattggaygtcactgtttcttacattttccgtt

SEQ ID NO: 249 - DNA, 51 bp, Homo sapiens
cttctaaaacatatcatgttcgattyaaggaataccattaagtaatatttt

SEQ ID NO: 250 - DNA, 51 bp, Homo sapiens
tgcatttgattcagttacaagtccartagttctaagccgggagtggaccct

SEQ ID NO: 251 - DNA, 51 bp, Homo sapiens
tttgttacttacagctgaattaagaygtttataaattatggctaaaaatta

SEQ ID NO: 252 - DNA, 51 bp, Homo sapiens
atcaaccaccaaagtcattattcaarcatagtggttctcaacggggtgatt

SEQ ID NO: 253 - DNA, 51 bp, Homo sapiens
aaacaatgagacatccagtaaaaccraatgcgataacaggaagagatcctc

SEQ ID NO: 254 - DNA, 51 bp, Homo sapiens
ccacctactaacaattccttgaacamagttaaatagtttttaaaagcagcc

SEQ ID NO: 255 - DNA, 51 bp, Homo sapiens
tactccacgacccaggctcattctayctaactacatagccagcttttctgg

SEQ ID NO: 256 - DNA, 51 bp, Homo sapiens
atccagctgttgtccatttccagaaraggagtgaatagtggcttaacagag

SEQ ID NO: 257 - DNA, 51 bp, Homo sapiens
ttaaatgagattggtgaatatcaaastgtttccccactgtaagaggctgta

SEQ ID NO: 258 - DNA, 51 bp, Homo sapiens
tttcagctttagtgttaatcatgtaractgtaccaaaattgttctcttcga

SEQ ID NO: 259 - DNA, 51 bp, Homo sapiens
cgtttttagtcttttaaggaagagayaaagccgtggtctacattttcatca

SEQ ID NO: 260 - DNA, 51 bp, Homo sapiens
attggaaaaagtttgaaaacaatccyggataataaatcacacttactcagc

SEQ ID NO: 261 - DNA, 51 bp, Homo sapiens
ggttccctggagagcataccttgtascaaagcatggcagtgagatgcagat

SEQ ID NO: 262 - DNA, 51 bp, Homo sapiens
aagagcccttctactgctatcacacyattctgagatttaggaaaccccatt

SEQ ID NO: 263 - DNA, 51 bp, Homo sapiens
tgctctctctcatatcttaaagccasgcaagtcaaatccaaattctttatc

SEQ ID NO: 264 - DNA, 51 bp, Homo sapiens
aatattttaagctgaaatcatgacamggttttacagttcattatattttac

SEQ ID NO: 265 - DNA, 51 bp, Homo sapiens
tcacattgtatcttaatcctagatcyaattttgcacccttgcccagcctag

SEQ ID NO: 266 - DNA, 51 bp, Homo sapiens
ggtaaatctagaaaaactggcgtaaytaacctcagctgatattctacagtg

SEQ ID NO: 267 - DNA, 51 bp, Homo sapiens
caatgaacgtatgcattttatttcakgtatttgtgataaataccttgaaat

SEQ ID NO: 268 - DNA, 51 bp, Homo sapiens
tgttctaacagaatataatgtgtccrgcaataattatatccaggtcaaata

SEQ ID NO: 269 - DNA, 51 bp, Homo sapiens
ttactgcctagcttcaattaattcaytctcaaggatctaaataaaaataaa

SEQ ID NO: 270 - DNA, 51 bp, Homo sapiens
taagaaattttccaaaaaaaattagycaatccttgccataaaaattatctg

SEQ ID NO: 271 - DNA, 51 bp, Homo sapiens
tctattgtcaaaaatgtcactgtgargctcttggttgcaccatcatctgca

SEQ ID NO: 272 - DNA, 51 bp, Homo sapiens
agcacattctatcaaaatccagtatracaaagtacaccttcttcttaacag

SEQ ID NO: 273 - DNA, 51 bp, Homo sapiens
actactttggatactggtagttgcaytaggataatcagaatgggtttggag

SEQ ID NO: 274 - DNA, 51 bp, Homo sapiens
ttggatgcaggcttccatttcattaracaagaatagggcccaaatattttt

SEQ ID NO: 275 - DNA, 51 bp, Homo sapiens
aaaactcaatcaatacattcgaaacrcagatattttgcttactgaggcttg

SEQ ID NO: 276 - DNA, 51 bp, Homo sapiens
tctagagacatgaggatactgataasatttttttgactttgcctttcaggg

SEQ ID NO: 277 - DNA, 51 bp, Homo sapiens
tgtcagagagattgttattcctgaartatctcagtttatgtaacccaaaaa

SEQ ID NO: 278 - DNA, 51 bp, Homo sapiens
gctgtgaacattcattcaaacgtacygatcatgttttattcctatgagtaa

SEQ ID NO: 279 - DNA, 51 bp, Homo sapiens
agaatccttgaattcatgaacctagrctaggacttgacaaactgtagctca

SEQ ID NO: 280 - DNA, 51 bp, Homo sapiens
ggtttctggtactaatagattctcawgttgcatagctctcgtgacctggct

SEQ ID NO: 281 - DNA, 51 bp, Homo sapiens
ccactcttgactgagctctctaaggrcacagactatgtactaatcagctca

SEQ ID NO: 282 - DNA, 51 bp, Homo sapiens
tatacaacctctgactgagcattagkccccatcatcagattctaggcctga

SEQ ID NO: 283 - DNA, 51 bp, Homo sapiens
ataccacctgggatcatactgaaaaygtttagaagccaagtcctgataact

SEQ ID NO: 284 - DNA, 51 bp, Homo sapiens
gctgtgtacaaatttaacatttttamccatggcaagacatctagagtaact

SEQ ID NO: 285 - DNA, 51 bp, Homo sapiens
gtaatccttttattggcttctattaygagtacaaaaaagtactgaaaatta

SEQ ID NO: 286 - DNA, 51 bp, Homo sapiens
ttaagccaacaagattttgcttacawccctagtaggaaagtaaccttgaaa

SEQ ID NO: 287 - DNA, 51 bp, Homo sapiens
ggacccaatagagttatgtcaaaaaytatggatgtgtaagaaccaggcagc

SEQ ID NO: 288 - DNA, 51 bp, Homo sapiens
ttggccagaaaggtttacagtgcaakgtactgtcttctatgtatttgactc

SEQ ID NO: 289 - DNA, 51 bp, Homo sapiens
tgcaggctcaaggtaaaattcaggasgtgatatccactaacagagaggtca

SEQ ID NO: 290 - DNA, 51 bp, Homo sapiens
ataaaaataaatcaagtaaaagagakgaacaaaattttgagacttgcatgg

SEQ ID NO: 291 - DNA, 51 bp, Homo sapiens
gctcttggccttatttcatgttcccrctttctaagcacattaggttttcct

SEQ ID NO: 292 - DNA, 51 bp, Homo sapiens
tgtaggaggtgacagttaaatgctayagtctttgatagattggtgtcaaga

SEQ ID NO: 293 - DNA, 51 bp, Homo sapiens
gtgatgatgatgatttccaaacagaktaggctgaattcatacacataagtg

SEQ ID NO: 294 - DNA, 51 bp, Homo sapiens
actttaacttccaatctcggttacaragagtgttaaaactggaagaaataa

SEQ ID NO: 295 - DNA, 51 bp, Homo sapiens
tataaatatggagcaaagatatcaarcataccagtaatgcttgctgaatac

SEQ ID NO: 296 - DNA, 51 bp, Homo sapiens
cgggttactataagaagttccagtamcacagtcttcctgcatgtgggctca

SEQ ID NO: 297 - DNA, 51 bp, Homo sapiens
agcaacttcagctaccaatgattatyaactctccttttaaaaaactgaaat

SEQ ID NO: 298 - DNA, 51 bp, Homo sapiens
atcgtgttcaaggtttgctgttatayggacccgatgtcaagcttggagaaa

SEQ ID NO: 299 - DNA, 51 bp, Homo sapiens
gcccaaccatcaaaataaagatgcaytcttaactgcagtcatgcaagttat

SEQ ID NO: 300 - DNA, 51 bp, Homo sapiens
tcatatttacaaaaaggatcgtgtcsaaaacatctccttttacttgtactt

SEQ ID NO: 301 - DNA, 51 bp, Homo sapiens
gaaataatttagtccaacctgctaamgtaatgccggaaatctttcgataaa

SEQ ID NO: 302 - DNA, 51 bp, Homo sapiens
tgttggttgggcaggtgtaataccarggtgtgtcatcgtcatcatggtttg

SEQ ID NO: 303 - DNA, 51 bp, Homo sapiens
aaaacacctaagtggatatgaaaaasggcatagaaattgaaatcagaagaa

SEQ ID NO: 304 - DNA, 51 bp, Homo sapiens
atcatgagattgtggaactactaacrgaaagctgtgatttgaacttggctc

SEQ ID NO: 305 - DNA, 51 bp, Homo sapiens
aagtcattttctccctgtaagttagragtagagaatacagagggggcttag

SEQ ID NO: 306 - DNA, 51 bp, Homo sapiens
agaataacttccatatataagagtaygctagaaatatatactcaaaaagta

SEQ ID NO: 307 - DNA, 51 bp, Homo sapiens
attcctctgtgttcagaactttacawaataataacagtctgagacaaacaa

SEQ ID NO: 308 - DNA, 51 bp, Homo sapiens
ttgaaggaagtagtgatggtggaaamttatgattcaactcatttctagaca

SEQ ID NO: 309 - DNA, 51 bp, Homo sapiens
cagctgagctttgcctcatagaatckctcttaggtgtcatccagtcccatc

SEQ ID NO: 310 - DNA, 51 bp, Homo sapiens
catttctttatatctaacccacagaytaagctctattctgggtgctgtgaa

SEQ ID NO: 311 - DNA, 51 bp, Homo sapiens
ttcaagggttttctatatattctcastcagtatgtgttaactggatgctca

SEQ ID NO: 312 - DNA, 51 bp, Homo sapiens
gaatacaaagtggttagatccaaaaygtgagctaggaactaaattcaataa

SEQ ID NO: 313 - DNA, 51 bp, Homo sapiens
atctattgaacttccagaaatataaractagcccaaggtctccagtttttt

SEQ ID NO: 314 - DNA, 51 bp, Homo sapiens
ttctgactttgaagtaatggatgaaytgtcatgtgcagtatatggaaataa

SEQ ID NO: 315 - DNA, 51 bp, Homo sapiens
ttcatttaaatgctcttgactatcasggaagataaggtgttccataagggc

SEQ ID NO: 316 - DNA, 51 bp, Homo sapiens
tatgacactataaattgttcattcaygctaaactctagtgaatgttgtcaa

SEQ ID NO: 317 - DNA, 51 bp, Homo sapiens
ccaatttttgtgcctcctactgttaygcccatttgattcttcaaactaagc

SEQ ID NO: 318 - DNA, 51 bp, Homo sapiens
cttggtaactgttaacaatagtcctsaatttgatgatcctaagttttggtt

SEQ ID NO: 319 - DNA, 51 bp, Homo sapiens
gttcctggttactgagctagaagatratcagaatgacaggcatgtggtgtc

SEQ ID NO: 320 - DNA, 51 bp, Homo sapiens
tcaaggaaactggcagatgtatataraaagataatgactcatctaataatt

SEQ ID NO: 321 - DNA, 51 bp, Homo sapiens
tttttatgtaagcctatgatagagakaaaacattttgagaaattagactga

SEQ ID NO: 322 - DNA, 51 bp, Homo sapiens
ctaacttatatgagatttttctgcartaagtctctatgtacttcctctggt

SEQ ID NO: 323 - DNA, 51 bp, Homo sapiens
ctaggagtatatatcacatttaagcratttaagaaatatctgtgacatgaa

SEQ ID NO: 324 - DNA, 51 bp, Homo sapiens
ttagtacaaaatcattacattaccgsaggctcatcattgctgaggaagcta

SEQ ID NO: 325 - DNA, 51 bp, Homo sapiens
gtaatgatgcaatatgatcctaagaygcggaaatattactaacatagatta

SEQ ID NO: 326 - DNA, 51 bp, Homo sapiens
cagtagcaatccagtgaataccaggwagataacttcaagtgatttaatata

SEQ ID NO: 327 - DNA, 51 bp, Homo sapiens
ggaaagttgaataaacaaggatatcrgaggctcagacaaacgggaaagatg

SEQ ID NO: 328 - DNA, 51 bp, Homo sapiens
acctcagctctctggacaggactacygggtttgtagatccaggttggactt

SEQ ID NO: 329 - DNA, 51 bp, Homo sapiens
gtgcctgcacagaatgggccctcaaygttgatttattgaatgaacacatga

SEQ ID NO: 330 - DNA, 51 bp, Homo sapiens
ccccatgactaatgactcttccacaytggacagcctttttagcagcctcca

SEQ ID NO: 331 - DNA, 51 bp, Homo sapiens
ttgattaagtctatatcacaatacartacagtcaaatttctcttctttttt

SEQ ID NO: 332 - DNA, 51 bp, Homo sapiens
tttgataaatagaagttcaaatacaygcagaaggattgtagctgagtagcc

SEQ ID NO: 333 - DNA, 51 bp, Homo sapiens
ctctgtgctatgttctcctcccggtyacagtttgcccatctcaaagttctt

SEQ ID NO: 334 - DNA, 51 bp, Homo sapiens
tgttctatcttaaaaagtcgtggcawcctgttacatgatctccttctattt

SEQ ID NO: 335 - DNA, 51 bp, Homo sapiens
gcacgactaaatcctaaacacatackatgaggcaaagagaaatatccagtg

SEQ ID NO: 336 - DNA, 51 bp, Homo sapiens
gctctcccagtcagagaaggcaggasagataatccactcaaccagaccaag

SEQ ID NO: 337 - DNA, 51 bp, Homo sapiens
cctcggaactcaaagttaggagatcraggccagaagttcatgtcattcccg

SEQ ID NO: 338 - DNA, 51 bp, Homo sapiens
tttgccagggtgtatgcataaaaaastttcataaaccttggatgccaggcc

SEQ ID NO: 339 - DNA, 51 bp, Homo sapiens
caaagtcttctagagctttggctaayaagagcagaaatctgaatccttgcc

SEQ ID NO: 340 - DNA, 51 bp, Homo sapiens
ttgtttctgtcaccactcgttccacrgagaatttaaggaaaattacaacaa

SEQ ID NO: 341 - DNA, 51 bp, Homo sapiens
tggaattttctttcccttccactcasgatatacaactaaaaacattgtcaa

SEQ ID NO: 342 - DNA, 51 bp, Homo sapiens
actgcatataactggttatctgaaayctcaaagcagtgccatctctctccc

SEQ ID NO: 343 - DNA, 51 bp, Homo sapiens
cctatagctaaactatcttacagccyactttgagtttgctaagctgctact

SEQ ID NO: 344 - DNA, 51 bp, Homo sapiens
tgtttagcctttgggtgctaagtaaygatagctctaatgagtcttatgctg

SEQ ID NO: 345 - DNA, 51 bp, Homo sapiens
agtccataaaataatttaccagtacrcagtctatatatactacctcagaga

SEQ ID NO: 346 - DNA, 51 bp, Homo sapiens
agagcttctgtcctgtagaatccaawccaaccaaagtggtgccatattccc

SEQ ID NO: 347 - DNA, 51 bp, Homo sapiens
taggatattcatgcctttcctataayaatgttttctgaagttatgtaatat

SEQ ID NO: 348 - DNA, 51 bp, Homo sapiens
caatcaaaggacttctaattcatgcwcttatcgtctgaccgaaataaagcc

SEQ ID NO: 349 - DNA, 51 bp, Homo sapiens
gggacaacttcaagtagttttgtgayggaagtaaattaaatgctgaggtga

SEQ ID NO: 350 - DNA, 51 bp, Homo sapiens
ctgttcctaggttggtgtcctcacaratgctgctgtttcaggcaatgtcag

SEQ ID NO: 351 - DNA, 51 bp, Homo sapiens
caaaacaagctgtgagaggctctgasaacagtgcattgtgggagaagtctg

SEQ ID NO: 352 - DNA, 51 bp, Homo sapiens
taccttggtcattataccatatgaartcatttctggtgagttttttatcta

SEQ ID NO: 353 - DNA, 51 bp, Homo sapiens
ctgagagatgtagtttatgcccaggwcactttgcttatggtcttctagctg

SEQ ID NO: 354 - DNA, 51 bp, Homo sapiens
agttctctctaggttcaggaggttawgattccagttctacttctggcccat

SEQ ID NO: 355 - DNA, 51 bp, Homo sapiens
cctcaatttgtagaaaaagattgatyaggaaggtccctgtccctgtaggtg

SEQ ID NO: 356 - DNA, 51 bp, Homo sapiens
agaaatctagaccaagaacctgtaaygttataactaaacacaccattccat

SEQ ID NO: 357 - DNA, 51 bp, Homo sapiens
agatccattctctacccaaatctacycatttctactctttttttctcagag

SEQ ID NO: 358 - DNA, 51 bp, Homo sapiens
aacataagcttggtggattatcgaamgacctcaaatgggtcaggagttcga

SEQ ID NO: 359 - DNA, 51 bp, Homo sapiens
ttagtaagtgtagactggaatcccarcttgaccattgtccgacttcagggc

SEQ ID NO: 360 - DNA, 51 bp, Homo sapiens
aaataaggagttatgattgaatcacragtttatttaattagagatttttga

SEQ ID NO: 361 - DNA, 51 bp, Homo sapiens
gaatggcttagagtggtacctggcaygtggtatgtttcttttttagacatg

SEQ ID NO: 362 - DNA, 51 bp, Homo sapiens
atcaagtccagcaaagctgctagacrctttctggaaggaaatatggccagg

SEQ ID NO: 363 - DNA, 51 bp, Homo sapiens
tcccttaagagtacatacataacaaytgaaccaaaagacattcttagatga

SEQ ID NO: 364 - DNA, 51 bp, Homo sapiens
actcttctttcatgatagggattaakgcaaaaatgaaatataaggcaacat

SEQ ID NO: 365 - DNA, 51 bp, Homo sapiens
tggataataaaattgtaatggatgcraagaagtgattactaggaaagccag

SEQ ID NO: 366 - DNA, 51 bp, Homo sapiens
gaaggctccgactatagaaggaagartatcatgtcaagcattctaaggatg

SEQ ID NO: 367 - DNA, 51 bp, Homo sapiens
attaccaacctaacaataaagaagcyaagcctagaacagtggttatgttga

SEQ ID NO: 368 - DNA, 51 bp, Homo sapiens
acatctcagaaaacttctaccaaaaygacagaaaagataaataatatgcag

SEQ ID NO: 369 - DNA, 51 bp, Homo sapiens
ctgcgatatcaggactaagtaccggsaaccaggatgctagacaaagtggtc

SEQ ID NO: 370 - DNA, 51 bp, Homo sapiens
gcctgaatgacccaccatgaaataartctaactgttgctaagctatctata

SEQ ID NO: 371 - DNA, 51 bp, Homo sapiens
tacgtgaaaaccaccctcagagtaaraacttcatctagggttgagatgtat

SEQ ID NO: 372 - DNA, 51 bp, Homo sapiens
gctgtcatggagaaagaggaggtcartatttttgttaactttgttataata

SEQ ID NO: 373 - DNA, 51 bp, Homo sapiens
tatttgccttcaaagtgaaggtccartagaagttttgtttatcacaaatca

SEQ ID NO: 374 - DNA, 51 bp, Homo sapiens
tgggaaaaattataatagcatttacygaaggatccaaatttttttaaataa

SEQ ID NO: 375 - DNA, 51 bp, Homo sapiens
aagggcccagaatttactgaatgccrctcacatgccaggactgtgctgggc

SEQ ID NO: 376 - DNA, 51 bp, Homo sapiens
ctgagactaatctgattttagtttayatggaagctggtttatcctattagc

SEQ ID NO: 377 - DNA, 51 bp, Homo sapiens
tacttgatgagctctttaaagagaartgggtgatttgcagcaaaattctta

SEQ ID NO: 378 - DNA, 51 bp, Homo sapiens
catgtcaccaattttttttgccaccrcctcacaaatttagtaagtttttaa

SEQ ID NO: 379 - DNA, 51 bp, Homo sapiens
gaagactgtctctccatgccaagtamaagactgtctctccatgctagagtg

SEQ ID NO: 380 - DNA, 51 bp, Homo sapiens
cagtccctgaaatataacaatgagayggaactcctgtgtccctgagtttat

SEQ ID NO: 381 - DNA, 51 bp, Homo sapiens
aataaggttggggaccactgtattasgcacttaaattgtgaaatgtgatgt

SEQ ID NO: 382 - DNA, 51 bp, Homo sapiens
gacactagtcttggcccactctatasaagtaagatcttgctaggcttgtat

SEQ ID NO: 383 - DNA, 51 bp, Homo sapiens
gacatggtacgttaacattcctgtayagttatcatgatttgcactcatcat

SEQ ID NO: 384 - DNA, 51 bp, Homo sapiens
tctgccttcttcatctcctctcttgwattaatgtcccacaccttcattctg

SEQ ID NO: 385 - DNA, 51 bp, Homo sapiens
ctgaatgtaactcagtttcgatatamgtttttaaaattctgatttagcaaa

SEQ ID NO: 386 - DNA, 51 bp, Homo sapiens
gtaatttttctagcagggctgaatgkataggatgttcacgagcatattttg

SEQ ID NO: 387 - DNA, 51 bp, Homo sapiens
caacccgtcttcccacaaactatccragctttcttattaatttatggtttt

SEQ ID NO: 388 - DNA, 51 bp, Homo sapiens
cctcaagtatgttaaacttctgaacmaactacttaaaaaccaaagttacgt

SEQ ID NO: 389 - DNA, 51 bp, Homo sapiens
cagagtgtaacagcagcaggataaaygaagaagaaggtccagataaaagta

SEQ ID NO: 390 - DNA, 51 bp, Homo sapiens
aagaaggtccagataaaagtaagaayagattacaaagtgagggaatctcag

SEQ ID NO: 391 - DNA, 51 bp, Homo sapiens
aatacgtaaaatcaatgctaactaakgtgcacaaataattatctgaatgta

SEQ ID NO: 392 - DNA, 51 bp, Homo sapiens
ctcagggaaggctgatagatggagayccaaaggcttaagaaaggaaaactg

SEQ ID NO: 393 - DNA, 51 bp, Homo sapiens
atttcacatctttatcatgaatgtcrgaaactagcttcagagatactgaaa

SEQ ID NO: 394 - DNA, 51 bp, Homo sapiens
acatgatgcagttagtaattttaaayaggcatggttaggtcctgtacagat

SEQ ID NO: 395 - DNA, 51 bp, Homo sapiens
agcagagttccaacttccaaccttarccattatcccctactacattaatca

SEQ ID NO: 396 - DNA, 51 bp, Homo sapiens
agactccagttttcttctgtgtagayccttgtgtgaagccctgcactttga

SEQ ID NO: 397 - DNA, 51 bp, Homo sapiens
aaacattgcccagtagtgaggaagamaactactcccgataaagtgattttg

SEQ ID NO: 398 - DNA, 51 bp, Homo sapiens
gctaaaaactacgatgtagagttgaygtacagattactattttatggtatt

SEQ ID NO: 399 - DNA, 51 bp, Homo sapiens
attatgcagatacctggcacacagcratacaccaaaggtagataaaactaa

SEQ ID NO: 400 - DNA, 51 bp, Homo sapiens
atagagaagatctgtaagtaatccaytgagaaacatgttttagagtcacaa

SEQ ID NO: 401 - DNA, 51 bp, Homo sapiens
accagttataaaagtagaaaaacgasaagagctacagaaattatttcactg

SEQ ID NO: 402 - DNA, 51 bp, Homo sapiens
tgcttgtgccaaggcagtggcattcrcttaaggacataggtcagcccacta

SEQ ID NO: 403 - DNA, 51 bp, Homo sapiens
tctttgtagatttcacacttaatcaragagtactggttcaaagcctggtat

SEQ ID NO: 404 - DNA, 51 bp, Homo sapiens
tttcttcccatagtcaatgtatttamgagtgttgattttagaaagaaacat

SEQ ID NO: 405 - DNA, 51 bp, Homo sapiens
atgtatagaagcttgtaatagtaaayaatttggtaaacagcaaagagtaaa

SEQ ID NO: 406 - DNA, 51 bp, Homo sapiens
gcttttgaagcataagtgattacaayttggcaatggtgtcaggcagaactg

SEQ ID NO: 407 - DNA, 51 bp, Homo sapiens
ccactgtctttcaccagagatgaaaktaacaatgtactcaatgtcatctag

SEQ ID NO: 408 - DNA, 51 bp, Homo sapiens
aaagttcataatataaaaacttcaaycattttatgcacttgtagtaaagac

SEQ ID NO: 409 - DNA, 51 bp, Homo sapiens
ttttaaaaaatcctggcctgctgaaygaatagtttcttctgcaacatttgt

SEQ ID NO: 410 - DNA, 33 bp, Homo sapiens
atctttttggccattaygttattgtccagtttt

SEQ ID NO: 411 - DNA, 51 bp, Homo sapiens
gatagacatctttttggccattatgktattgtccagtttttggctattatg

SEQ ID NO: 412 - DNA, 51 bp, Homo sapiens
gaacaaagaaattatggcacattccracaattaaatactacctagcaataa

SEQ ID NO: 413 - DNA, 51 bp, Homo sapiens
ttacagagaataaacatttagattgktgtaagcctttgactaatttctaga

SEQ ID NO: 414 - DNA, 51 bp, Homo sapiens
attgaatggtaaatttagacctagartacaatgactggttgtttaagaaag

SEQ ID NO: 415 - DNA, 51 bp, Homo sapiens
ctgaagtacaggaaatgggctacacmactgggacactgaattctgaaatgt

SEQ ID NO: 416 - DNA, 51 bp, Homo sapiens
ttcaataaaattcaacttgacttcayggtaaaaatccttaacaaattatat

SEQ ID NO: 417 - DNA, 51 bp, Homo sapiens
gaagcttatagtctaaactgaggatyattcacctgaagagattcctatggt

SEQ ID NO: 418 - DNA, 51 bp, Homo sapiens
ggtgcagggtatcagtggtaataggmggtgcatctgagtcatcgtggcttg

SEQ ID NO: 419 - DNA, 51 bp, Homo sapiens
ttttcttattaaattgtttgatctcraatctggaagcacgcccaagatata

SEQ ID NO: 420 - DNA, 51 bp, Homo sapiens
atctccaaaccttacagacaccctgmagtataggtatgctcatcctcatag

SEQ ID NO: 421 - DNA, 51 bp, Homo sapiens
atgaattctggcagagtatataccaytgagatacaatagatgcaggaatct

SEQ ID NO: 422 - DNA, 51 bp, Homo sapiens
aggacacatagctatttcttataaamggaatcatttaagtgtatgttgcct

SEQ ID NO: 423 - DNA, 51 bp, Homo sapiens
ttgcaaagctagggaaacataaatarggaatcatggcttgagcttttcaat

SEQ ID NO: 424 - DNA, 51 bp, Homo sapiens
ggatacagagccacaccatatcaaakgtcttcttcatatattttaaaatca

SEQ ID NO: 425 - DNA, 51 bp, Homo sapiens
tttgtctttcccaccttgttaggctkcttctttgcctttctgcctggcaat

SEQ ID NO: 426 - DNA, 51 bp, Homo sapiens
tgattaattgaatgacttcaggagarcattttttctcaatttcttacactg

SEQ ID NO: 427 - DNA, 51 bp, Homo sapiens
cctgggacaaagataggatcttacarctttccagagaggaacaaccaaccc

SEQ ID NO: 428 - DNA, 51 bp, Homo sapiens
gatgacagactttcagttaaactaayaagtaagtcaactcatggtcaacag

SEQ ID NO: 429 - DNA, 51 bp, Homo sapiens
gaaacatgtttatttttattatttarggttccaaagactggggtaggtaaa

SEQ ID NO: 430 - DNA, 51 bp, Homo sapiens
aaagcagcatgccaaaaagcctccartaagcaggtgaagtgattaagttgt

SEQ ID NO: 431 - DNA, 51 bp, Homo sapiens
tgtgccacaatcatcaaagatagttwaaagtgccagagttgggagtttcag

SEQ ID NO: 432 - DNA, 51 bp, Homo sapiens
ctacagggaggggagtagaatcttayggcccagcattggcattgaggcatc

SEQ ID NO: 433 - DNA, 51 bp, Homo sapiens
tttatggatgtatgtgtttaataacrataaaatgcatgcatagaagtgata

SEQ ID NO: 434 - DNA, 51 bp, Homo sapiens
aggtataagacagtaaacactgcaartgcctaaagatagtgaggaaatgtg

SEQ ID NO: 435 - DNA, 51 bp, Homo sapiens
ccaagcacaaggttcactatgactaygcgactattgtaacaaagctaaaga

SEQ ID NO: 436 - DNA, 51 bp, Homo sapiens
ctgtgtggggatatttcacaaagagrcatggtgaagtgcggcactgcctac

SEQ ID NO: 437 - DNA, 51 bp, Homo sapiens
gaaggtgatggaataattttaagaarcaagaatctgaatgaagtctggatt

SEQ ID NO: 438 - DNA, 51 bp, Homo sapiens
ggatctatgatctggaaataggtcartacatatgtctgttataaagaagag

SEQ ID NO: 439 - DNA, 51 bp, Homo sapiens
gaaatctaattatgtgccaagcaccrgactaagtacttttcacgttatttc

SEQ ID NO: 440 - DNA, 51 bp, Homo sapiens
aacaaatccagaaggagtgcaattaycaacaccactctccttctgctattg

SEQ ID NO: 441 - DNA, 51 bp, Homo sapiens
tttcttgattgtccattataattccrtatgttgtatcttctgctgtagtat

SEQ ID NO: 442 - DNA, 51 bp, Homo sapiens
tgtttcgtttatgaaacaaacaatgraatatttagttaaaccaggcaacag

SEQ ID NO: 443 - DNA, 51 bp, Homo sapiens
tattttcctcaaaataaccctaggcrataactactactatttttcccattt

SEQ ID NO: 444 - DNA, 51 bp, Homo sapiens
taaaggagttgtattagcttggaatsaaagggacagttgctatataaaagg

SEQ ID NO: 445 - DNA, 51 bp, Homo sapiens
gttgaggattctcctgataggtactraaaggttatactaaatgaaagtaaa

SEQ ID NO: 446 - DNA, 51 bp, Homo sapiens
taaatatgctcatcttggggcaggaygttctccttctgtaagtctctagag

SEQ ID NO: 447 - DNA, 51 bp, Homo sapiens
aagtattctgattctgcggggattasagttttgaagtgttattctgtcatc

SEQ ID NO: 448 - DNA, 51 bp, Homo sapiens
aaaatctcaagcccttgtatgaaacraagaagtgtggcctgtccctacgta

SEQ ID NO: 449 - DNA, 51 bp, Homo sapiens
taccaaatggagctcactgttcagaytcgttgcaaaatctggctaaaattt

SEQ ID NO: 450 - DNA, 51 bp, Homo sapiens
ctaggcatgaaggtaggaaatagcayggtgagtaagaaatatgtttgcagt

SEQ ID NO: 451 - DNA, 51 bp, Homo sapiens
tctcttatgatgaggaaagtttgaasccagtgacaatcatgcttcagatga

SEQ ID NO: 452 - DNA, 51 bp, Homo sapiens
atctggggagaattttgaaaataggkactcctcatcttcaccatcagttcc

SEQ ID NO: 453 - DNA, 51 bp, Homo sapiens
caaatacacctcttaacaatcaaaartcatttaatacaatttttcccaaaa

SEQ ID NO: 454 - DNA, 51 bp, Homo sapiens
gtggagagggcaccaaggtcaaagtwtgtctcctcatagctttgcagaggg

SEQ ID NO: 455 - DNA, 51 bp, Homo sapiens
acagatattgactacctcttctctgwgcttagaattgcatttaagtggcag

SEQ ID NO: 456 - DNA, 51 bp, Homo sapiens
tttttcaagatactcagcatgtatamagtatatctgcaatccattttctgt

SEQ ID NO: 457 - DNA, 51 bp, Homo sapiens
agggttgtatcacccggctgacagayaaatataaagtaaaacctcagatgc

SEQ ID NO: 458 - DNA, 51 bp, Homo sapiens
acattaattaaactgtttgagaagastcaccctctaatcttttttgaaaac

SEQ ID NO: 459 - DNA, 51 bp, Homo sapiens
gaaatctaccatcattaccaagctcratgaataaatgatatagatatggca

SEQ ID NO: 460 - DNA, 51 bp, Homo sapiens
gatgagcctggatctccagaagataytaggaatatagagtcagctggtagg

SEQ ID NO: 461 - DNA, 51 bp, Homo sapiens
atttaagggtgggcaacttcataacraacccgagccaggaccacttagcta

SEQ ID NO: 462 - DNA, 51 bp, Homo sapiens
ggtctttccccaaagcatctctctaygtcaataatagaccctatccttgga

SEQ ID NO: 463 - DNA, 51 bp, Homo sapiens
catttaacctttgtgtggtgctaacmattagtgatcctataactaacaaaa

SEQ ID NO: 464 - DNA, 51 bp, Homo sapiens
atactcaaaagtctgagaataagaargagttttgggaagttaaaaaatgtt

SEQ ID NO: 465 - DNA, 51 bp, Homo sapiens
ttgactcagtacaagtgtgctttaamtagtctctccagaactggttcacct

SEQ ID NO: 466 - DNA, 51 bp, Homo sapiens
ggtatggaaaatacgtataaggtccmaaatcacacttttgatcatagaatt

SEQ ID NO: 467 - DNA, 51 bp, Homo sapiens
gattaagagaacattctgtacctgaygtccaaaaaacaaagaaccctgtag

SEQ ID NO: 468 - DNA, 51 bp, Homo sapiens
cctgccaaggtaaactagttcatacrgcttatgtgacatcctttgggatct

SEQ ID NO: 469 - DNA, 51 bp, Homo sapiens
taatttataaaaggctctcaagtcartatcaagaaaaactaacccaaatac

SEQ ID NO: 470 - DNA, 51 bp, Homo sapiens
cctttggctgattttcctaatttatrttgcacttacaacacctgacacgaa

SEQ ID NO: 471 - DNA, 51 bp, Homo sapiens
acacgttcacagccagctacaggagmgtttgggtcagcctattaagcacat

SEQ ID NO: 472 - DNA, 51 bp, Homo sapiens
gtgtgccatagacacagtcttcacayagatttggcaaaccatcctagttta

SEQ ID NO: 473 - DNA, 51 bp, Homo sapiens
tatgaggtattatgttgatgcaaacratgactaacccttcaaggacattcc

SEQ ID NO: 474 - DNA, 51 bp, Homo sapiens
cattttgcaatgttaaatcatgtacyacttctcaaccctttaaggtaagtg

SEQ ID NO: 475 - DNA, 51 bp, Homo sapiens
agtcccctagtcttcaaccaaatccratgaataaccaagccctgtggattt

SEQ ID NO: 476 - DNA, 51 bp, Homo sapiens
cacctcccccgattcaaacttgaaaytaagtatgagctaaattccctgtta

SEQ ID NO: 477 - DNA, 51 bp, Homo sapiens
ctatctcagattttcaagaatcctcrtacaaattgtccagggtttccacct

SEQ ID NO: 478 - DNA, 51 bp, Homo sapiens
tcacttaacattgggttatcaagtayatgtcagatatctttccctggatct

SEQ ID NO: 479 - DNA, 51 bp, Homo sapiens
gcgacaaagtgtggttatggtaagtyttgagagaaatgagcatccattttc

SEQ ID NO: 480 - DNA, 51 bp, Homo sapiens
catccttctaaaatacacaccttaamacctgctgatgacttcctatggcct

SEQ ID NO: 481 - DNA, 51 bp, Homo sapiens
ggtggatgggtgcagcaaacaattaygacacttgtctacctatgtaacaaa

SEQ ID NO: 482 - DNA, 51 bp, Homo sapiens
cagccatccccacaactgctttgaargacataggattaattttacagtcag

SEQ ID NO: 483 - DNA, 51 bp, Homo sapiens
tcgataacacaccattgagtgaattsattacccaatcctggatctgcccta

SEQ ID NO: 484 - DNA, 51 bp, Homo sapiens
agagacaatcttaaagcaactagaargaatacatcaaaacagaccaacaat

SEQ ID NO: 485 - DNA, 51 bp, Homo sapiens
ttccttggcctttcaggaacatctcracacaggtgaacaccttctctttct

SEQ ID NO: 486 - DNA, 51 bp, Homo sapiens
aatctagcctgatttttgtggtcaaratcacccttacttatctctgaaaat

SEQ ID NO: 487 - DNA, 51 bp, Homo sapiens
tcactgtaggcctgccttgcactcaycaagtcctggagaggactaaatact

SEQ ID NO: 488 - DNA, 51 bp, Homo sapiens
gggtgggtcctctggaggaaaaatcrtctttccaaatggaaagatgaatga

SEQ ID NO: 489 - DNA, 51 bp, Homo sapiens
agcttgtctataaaagctgagtatayctgagagtgatttcctgagtctaga

SEQ ID NO: 490 - DNA, 51 bp, Homo sapiens
tgacttctgagagaggaaacacctaygcaatccgctgacagtcttggttgg

SEQ ID NO: 491 - DNA, 51 bp, Homo sapiens
gtgttttcactctcttgcattttctycaatgaaaagctaagtacaggagtg

SEQ ID NO: 492 - DNA, 51 bp, Homo sapiens
agtatttatttattagatcaatttasagatatgcatctgtgtggccagaac

SEQ ID NO: 493 - DNA, 51 bp, Homo sapiens
atagctgtgcaagacacccagaatasgagatgatctgactctgggtatgaa

SEQ ID NO: 494 - DNA, 51 bp, Homo sapiens
gttctctatatggtgattattcccaygctaaacccattcatttatagcttg

SEQ ID NO: 495 - DNA, 51 bp, Homo sapiens
agtagttgtggtagcacctacaatgkatcatgtatgtgaagaatcagaatt

SEQ ID NO: 496 - DNA, 51 bp, Homo sapiens
tggacagggacctgacagaggatatmaccaagctcagatcccagaggcttg

SEQ ID NO: 497 - DNA, 51 bp, Homo sapiens
ctttaaaaaaacatcatttacttttsgtcatgctggatttggggcattcct

SEQ ID NO: 498 - DNA, 51 bp, Homo sapiens
caaaagtatatcattagatttagaayaaacgtactgtacatcctggtttca

SEQ ID NO: 499 - DNA, 51 bp, Homo sapiens
ataatgctgtgactttgtattagccrggagtcctgaattatcaaaagattt

SEQ ID NO: 500 - DNA, 51 bp, Homo sapiens
ataaaacggctttcttatattgaaartactgggtgatgtagttgaaatcag

SEQ ID NO: 501 - DNA, 51 bp, Homo sapiens
tcattgccctgattggttgcctgatraaacagttcagtcacaatggctgag

SEQ ID NO: 502 - DNA, 51 bp, Homo sapiens
tctctaacccttacgtttatggcaayttcttctacatttcccatgcaagca

SEQ ID NO: 503 - DNA, 51 bp, Homo sapiens
tatgattattaagctgaaaattataraagggtatatacatactttacctgt

SEQ ID NO: 504 - DNA, 51 bp, Homo sapiens
cccacactgattcttggtttagtcaygtaacttgctttaacctccaacaga

SEQ ID NO: 505 - DNA, 51 bp, Homo sapiens
caagacagcttaaaacataaagaaargttgacactgaaaaaggtaatagat

SEQ ID NO: 506 - DNA, 51 bp, Homo sapiens
tgattgctttagtgcaccttatatamctttgagtctctatatttcaggctg

SEQ ID NO: 507 - DNA, 51 bp, Homo sapiens
aaaatatgtcaactcctgactgttgsagaaaatgaagggtcaaaaataaag

SEQ ID NO: 508 - DNA, 51 bp, Homo sapiens
gtctggaatacgaggcatttcagatmataagggtcatacttctacttttat

SEQ ID NO: 509 - DNA, 51 bp, Homo sapiens
atttcttgctagtattgctgaaatasacctctaataggttcctaaccttac

SEQ ID NO: 510 - DNA, 51 bp, Homo sapiens
tgaagattggattcataaactattayacatttgccaaggtatggaaagaag

SEQ ID NO: 511 - DNA, 51 bp, Homo sapiens
atgtctgatgttagatcacaaagaartgaaggtatgttatctaagaaattt

SEQ ID NO: 512 - DNA, 51 bp, Homo sapiens
gccaaattgataacttcctgggtagratatatactataggtcaattcctct

SEQ ID NO: 513 - DNA, 51 bp, Homo sapiens
aactaggttctcaacacatgttcaaytaactccagatatggaactcttcca

SEQ ID NO: 514 - DNA, 51 bp, Homo sapiens
attgactgggaatgagctttgcgtaygattgtgtttcatctaagttctgtt

SEQ ID NO: 515 - DNA, 51 bp, Homo sapiens
agagataaaaagaagatgagaataarataatatacctgtctaagaatagaa

SEQ ID NO: 516 - DNA, 51 bp, Homo sapiens
agttgtgaccttgaaatcccgcagartatctaccatcccttccagcttctg

SEQ ID NO: 517 - DNA, 51 bp, Homo sapiens
tttattaacttatttaattatcacarctattctgtatggagcactctgttt

SEQ ID NO: 518 - DNA, 51 bp, Homo sapiens
tgatttattctttcctcctctgaaaytcaacacggtagtaaacactgaacc

SEQ ID NO: 519 - DNA, 51 bp, Homo sapiens
aataagctctatatttttgaaatacsaagactaagatgtaaacttcgatct

SEQ ID NO: 520 - DNA, 51 bp, Homo sapiens
tttcactgctctactgtctactgcamgaaagagaaaataggtgtatgaaaa

SEQ ID NO: 521 - DNA, 51 bp, Homo sapiens
actattcttctttgtcaggcatttcrcagctttgtgtctcgggaatacaaa

SEQ ID NO: 522 - DNA, 51 bp, Homo sapiens
attgctgctacaaccttaaatataayggtgtgaatggctctgctattattt

SEQ ID NO: 523 - DNA, 51 bp, Homo sapiens
tgtctgatgttgtactgagcagataygaaggagtaggaaggagaaagaatg

SEQ ID NO: 524 - DNA, 51 bp, Homo sapiens
tgttttttgagattctgaatgcccaygttaagtaacaaacattcatctaga

SEQ ID NO: 525 - DNA, 51 bp, Homo sapiens
ctgctcatggataaaatgtcttcaayaaaatcggtccttggcaaccgctgc

SEQ ID NO: 526 - DNA, 51 bp, Homo sapiens
caagaaattgaaaatgacctaaaggracatcaataatggaatggaaaaata

SEQ ID NO: 527 - DNA, 51 bp, Homo sapiens
ccttgttattttctctagacattacyggactcactaacgcagtggcccgaa

SEQ ID NO: 528 - DNA, 51 bp, Homo sapiens
tttgacctcctttcccagtccatacrcttaacaattgttttccacccaact

SEQ ID NO: 529 - DNA, 51 bp, Homo sapiens
ggaccatatgtaatgcacattagaartattgtttaaggaatcattaaatca

SEQ ID NO: 530 - DNA, 51 bp, Homo sapiens
tttctagaatctgttcccttcaagcraccctgtaattgtgcattttgtttt

SEQ ID NO: 531 - DNA, 51 bp, Homo sapiens
gagtatcttataagtgaagtacaccmaatttagcctataactttttgttat

SEQ ID NO: 532 - DNA, 51 bp, Homo sapiens
aaaatgcatgttctgtatcagtctargtctaatcaagaaagagaaaccact

SEQ ID NO: 533 - DNA, 51 bp, Homo sapiens
gattaaatccaacaaagatttcccastctactacatgcttgttccatgctg

SEQ ID NO: 534 - DNA, 51 bp, Homo sapiens
tatcaagcaagtcatattagtgatgrttcaatgcattttttcattatatac

SEQ ID NO: 535 - DNA, 51 bp, Homo sapiens
ttaagcttgttgaagatagtggttaygtatttttatcaaagcactcgcctg

SEQ ID NO: 536 - DNA, 51 bp, Homo sapiens
atataaggtgatactaaggctcggcrtcctttaacctgtcattagtcaagg

SEQ ID NO: 537 - DNA, 51 bp, Homo sapiens
aagtgaagtttttctgattttaatcygtcaggtttccatgttctataatcg

SEQ ID NO: 538 - DNA, 51 bp, Homo sapiens
tctgtccattctaataattcaccaamgatagccattccccatagagagaga

SEQ ID NO: 539 - DNA, 51 bp, Homo sapiens
ctgttcagttcttggaaacacatgcyctttggaaaaatacctaaagagatg

SEQ ID NO: 540 - DNA, 51 bp, Homo sapiens
tgttttttgcctaatttaatatatargcaactgactaactacttctttggg

SEQ ID NO: 541 - DNA, 51 bp, Homo sapiens
acaaatatttttggagaatttcttaygtgttaggtgcagtgcaaggctctt

SEQ ID NO: 542 - DNA, 51 bp, Homo sapiens
atctattgacaaataaactgatttamattaagtgagtgctgcttcgtgtta

SEQ ID NO: 543 - DNA, 51 bp, Homo sapiens
ctatatgagatgttggtgatactcaygatatttccaattttttaattatta

SEQ ID NO: 544 - DNA, 51 bp, Homo sapiens
gctattagaaataaaattgataagcrcattcatgtacaattatttatgtgg

SEQ ID NO: 545 - DNA, 51 bp, Homo sapiens
ttctgatctggtttaaatttgttgayggcaattatgttatgtcatattgaa

SEQ ID NO: 546 - DNA, 51 bp, Homo sapiens
caatacatgctattttcactccacartatatttataaataatttctaatga

SEQ ID NO: 547 - DNA, 51 bp, Homo sapiens
ttcattcattcaacataaatgaaacyacatattcccttggcgtgaaattta

SEQ ID NO: 548 - DNA, 51 bp, Homo sapiens
gttctttgcacatactggatacctaygttgcaagtaacttctactattttg

SEQ ID NO: 549 - DNA, 51 bp, Homo sapiens
ggtgtttgaccaagcaattgtgcacygtaacccagggagattgatacataa

SEQ ID NO: 550 - DNA, 51 bp, Homo sapiens
aaagtccacagtggcctgtggacatraacataactctcagagacaacactc

SEQ ID NO: 551 - DNA, 33 bp, Homo sapiens
ctcttgaatggaaagcrtttaattgttcacacc

SEQ ID NO: 552 - DNA, 51 bp, Homo sapiens
aaaaactagatcaatcaacccaaagratatgtctcaatccctgaagggtct

SEQ ID NO: 553 - DNA, 51 bp, Homo sapiens
taaacatattcaactgacaagaaaartatttaaaagtaataaaccccccct

SEQ ID NO: 554 - DNA, 51 bp, Homo sapiens
tagtaaaacaaaaaacaaaagtagakagtccagtttagaggaggaaaagta

SEQ ID NO: 555 - DNA, 51 bp, Homo sapiens
catgtgcctcctggcctatccgtccyagagttggaggaggaggagccccaa

SEQ ID NO: 556 - DNA, 51 bp, Homo sapiens
agaggacactaaggagaaataatgastaaatgcaatttgatatcctggatt

SEQ ID NO: 557 - DNA, 51 bp, Homo sapiens
gaaaagttatggttatctctcataaytgttgaaaagaataaaccagataag

SEQ ID NO: 558 - DNA, 51 bp, Homo sapiens
gcttttagagtatgtgttcttttaaytgataagatggtactattctcatga

SEQ ID NO: 559 - DNA, 51 bp, Homo sapiens
gattgggactggaactctaaggagakaatttcagcagagaagcgtgcccag

SEQ ID NO: 560 - DNA, 51 bp, Homo sapiens
tatctgttttcacttttcaagtaaawccctacatggtgatcataaaaacaa

SEQ ID NO: 561 - DNA, 51 bp, Homo sapiens
ttagtagtgattctaatttgttccayagataaaactaggggaatttcaaga

SEQ ID NO: 562 - DNA, 51 bp, Homo sapiens
actgggtgggcttctgtcaaaaaccrggcctctatcagaactacaatgcag

SEQ ID NO: 563 - DNA, 51 bp, Homo sapiens
ggagggtggactctgctgtgaagaaytccctgatttctgtagacaatgaat

SEQ ID NO: 564 - DNA, 51 bp, Homo sapiens
tcattggttttgaagaagcctttggractagataagaattcagggaaaggt

SEQ ID NO: 565 - DNA, 51 bp, Homo sapiens
gccctgggtaacactgtaacctccarggtagattgaataggaaaaacttaa

SEQ ID NO: 566 - DNA, 51 bp, Homo sapiens
ctgtaaacatagtgtaaagcaatgcraacgtgtatgtgaaatttggctcca

SEQ ID NO: 567 - DNA, 51 bp, Homo sapiens
ggtttcatcttctgttcaggccattraaactttctccctatcagcaataca

SEQ ID NO: 568 - DNA, 51 bp, Homo sapiens
acatcagtcctgccacattatggggratataaactgttaagttattccctg

SEQ ID NO: 569 - DNA, 51 bp, Homo sapiens
gctgtcctgtctctgagtagttgccrcagtagccttgctggtgccggaggc

SEQ ID NO: 570 - DNA, 51 bp, Homo sapiens
ggtgagtgtcctttgtgggccctgayaacaagatctgctcctctaaatttg

SEQ ID NO: 571 - DNA, 51 bp, Homo sapiens
gggaaatgaagaataattgcttaacrgatgtgggggttccatttagggtga

SEQ ID NO: 572 - DNA, 51 bp, Homo sapiens
ggctgaggagcaaccacttgatacasagatttgcatgactaaaaaacaacc

SEQ ID NO: 573 - DNA, 33 bp, Homo sapiens
gggaggttttgcaaaakctttgggagaggatta

SEQ ID NO: 574 - DNA, 51 bp, Homo sapiens
tattagatctcttaaaatcgtcctayagatcactaatgcttttctccattt

SEQ ID NO: 575 - DNA, 51 bp, Homo sapiens
attttcttgattctccatatgcctgracatttgattggatgcccaaaattg

SEQ ID NO: 576 - DNA, 51 bp, Homo sapiens
agtccaaaagctggaccttttctgawtgataaaaaagtgacacggacttat

SEQ ID NO: 577 - DNA, 51 bp, Homo sapiens
cccacttccttggaaacccgtgtgayacaacagaggcagccatgatccccc

SEQ ID NO: 578 - DNA, 33 bp, Homo sapiens
acctgccatcaatagcytagaaataagtttttg

SEQ ID NO: 579 - DNA, 51 bp, Homo sapiens
acggctgcaactgtgaggaaacatamaagagccacgcaactgagcaagagt

SEQ ID NO: 580 - DNA, 51 bp, Homo sapiens
caaagctggaaaacgagttataacckctgcaccttacaaacctgttcctgc

SEQ ID NO: 581 - DNA, 51 bp, Homo sapiens
aggttgcttcatgatcattgcccagractaaacatttttcaagatggattt

SEQ ID NO: 582 - DNA, 51 bp, Homo sapiens
tgagtaatcagctgggtgaatagaamcctgttaggcctaagaggcagccac

SEQ ID NO: 583 - DNA, 51 bp, Homo sapiens
taacttactttccctctttctatgarcaatggcaaataatctgtatggtta

SEQ ID NO: 584 - DNA, 51 bp, Homo sapiens
cccattccttcccatggaaattacartaaaagctctcgcccccattttccc

SEQ ID NO: 585 - DNA, 51 bp, Homo sapiens
ttaaaactctttgtacaggaatgaawgttaatcatcaatttaaacacaaca

SEQ ID NO: 586 - DNA, 51 bp, Homo sapiens
caaaataagatattatttgccaaaakacattgaaataacacaatataatcc

SEQ ID NO: 587 - DNA, 51 bp, Homo sapiens
ccagggaatgtgtcagatgcctaagraccttcacagcaattctagcaattc

SEQ ID NO: 588 - DNA, 51 bp, Homo sapiens
ctataaacattattaaccatctttcyaagaaaattcaatgatgataatgta

SEQ ID NO: 589 - DNA, 51 bp, Homo sapiens
gacaggcaagtggttgagggagcaamaatgagaccagacagagatgaaaaa

SEQ ID NO: 590 - DNA, 51 bp, Homo sapiens
catgttcattctagctcaccaagaartgcttgaaattataggtaaatataa

SEQ ID NO: 591 - DNA, 51 bp, Homo sapiens
gttaaagaagactgactattttacawccacaggtatgaaaatttaagttgg

SEQ ID NO: 592 - DNA, 51 bp, Homo sapiens
gaagcctatgcagaatgggtatagaktcaggagtttggagtgcagagggag

SEQ ID NO: 593 - DNA, 51 bp, Homo sapiens
ctcacagtatacccagataacaaccygcatattgtatacctcctaaatata

SEQ ID NO: 594 - DNA, 51 bp, Homo sapiens
ctttgtgcatttgctgtacatacacygtgaaaagccctgtgccagcccaaa

SEQ ID NO: 595 - DNA, 33 bp, Homo sapiens
ctagtgctattagattmcaacacgtgggctcta

SEQ ID NO: 596 - DNA, 51 bp, Homo sapiens
tattagtatcccatgggaaagagaaytgggacaacctcaacaatgagttgg

SEQ ID NO: 597 - DNA, 51 bp, Homo sapiens
gacaaaggatttagaaacactagaakaagaactctccttgaattcctaatg

SEQ ID NO: 598 - DNA, 51 bp, Homo sapiens
tgtcctttatgttgataaaattgcasaaactgctgtagtgcaatgtccttg

SEQ ID NO: 599 - DNA, 51 bp, Homo sapiens
aaaataacatcctgattaattttccyaacataaaaacactcatcattttag

SEQ ID NO: 600 - DNA, 51 bp, Homo sapiens
tgaaactaattgcactctaatcatgrgaataattgtaaagtctatcttcag

SEQ ID NO: 601 - DNA, 51 bp, Homo sapiens
agtagagtgagcccttaatctaatayaacatggatctttataagaaaagga

SEQ ID NO: 602 - DNA, 51 bp, Homo sapiens
tgtatctttcctataatttttctcaycaccctgaccaatctcctacatctc

SEQ ID NO: 603 - DNA, 51 bp, Homo sapiens
cacagatagaatagatttcacataaytcttaagggccctaggattttcgga

SEQ ID NO: 604 - DNA, 51 bp, Homo sapiens
tcctctggccactcagagagtcctaraaggaggaagtctgaaacagtggtt

SEQ ID NO: 605 - DNA, 51 bp, Homo sapiens
gaaacataaggttctaaggctcttgytggaaaccataatcagaactggagg

SEQ ID NO: 606 - DNA, 51 bp, Homo sapiens
agcccgagggaaaatgcaaagctggwcacagcgcagatggggagacctctg

SEQ ID NO: 607 - DNA, 51 bp, Homo sapiens
atataaatttatcttaaattcagtcrgagcggaaaaaacagattccctata

SEQ ID NO: 608 - DNA, 51 bp, Homo sapiens
cccggctaatttttgtatttttaatsaagttggagtgtcactatgttggcc

SEQ ID NO: 609 - DNA, 51 bp, Homo sapiens
tttttaaaatactgttcttttatcayagtcacctgagaactgctttagacc

SEQ ID NO: 610 - DNA, 51 bp, Homo sapiens
aaatgcattcattttaactcttccayctgttactactaccacaatcaagat

SEQ ID NO: 611 - DNA, 51 bp, Homo sapiens
ctactaagcctggctttctacaagarcttcattttatgcatcttttgtctt

SEQ ID NO: 612 - DNA, 51 bp, Homo sapiens
tgtttgtgaactttgaagacagttgsattgaatgagagtcacagatctgac

SEQ ID NO: 613 - DNA, 51 bp, Homo sapiens
accaaaattatgactgataacattgyaccagatttctaggaatagcatgtg

SEQ ID NO: 614 - DNA, 51 bp, Homo sapiens
ctttggtgccttgtaagtgatatcargttcgtagctcttgcagacatctca

SEQ ID NO: 615 - DNA, 51 bp, Homo sapiens
agtccctaccactatttagaaggatwacctgactcacatgaacaaagcaag

SEQ ID NO: 616 - DNA, 51 bp, Homo sapiens
cttcctttcctcccatttctgaatayggtggccctgttgtagaaaaagtgc

SEQ ID NO: 617 - DNA, 51 bp, Homo sapiens
tactatttgtggtatcaattcattcwcattgggtttactaaattattattt

SEQ ID NO: 618 - DNA, 51 bp, Homo sapiens
tgggatacccagatctatggtaaaaygttatttcagagtgtgtctgtgagg

SEQ ID NO: 619 - DNA, 51 bp, Homo sapiens
gctcttggttctcaggcttttggacyctaaggcttatatcaatagcctccc

SEQ ID NO: 620 - DNA, 51 bp, Homo sapiens
agagctttttagcataactcggagcrcataggcaatcaagaaaacattggg

SEQ ID NO: 621 - DNA, 51 bp, Homo sapiens
gttcccctctgacagccctgttggayggaactttgctttatagaggcatca

SEQ ID NO: 622 - DNA, 51 bp, Homo sapiens
ttcttgtttccaatattatgggacaygaaatctttgagattctagaaagtt

SEQ ID NO: 623 - DNA, 51 bp, Homo sapiens
tgcattttaaaaactataactccaawgctcatttgattgtggccctagtca

SEQ ID NO: 624 - DNA, 51 bp, Homo sapiens
gagaaaggaaatcatttatccactcrctttcacattttcattgatcaaata

SEQ ID NO: 625 - DNA, 51 bp, Homo sapiens
acacagctttctatgaaaaatgaaarataaaagttgtagagaccaagggaa

SEQ ID NO: 626 - DNA, 51 bp, Homo sapiens
agcggaatgaaaatagctaacatcamaatcagtgaccagaaaagaaagcca

SEQ ID NO: 627 - DNA, 51 bp, Homo sapiens
gcatgttcataattgcccattgaaasattttttacatggctactctaaaat

SEQ ID NO: 628 - DNA, 51 bp, Homo sapiens
aacagctgcctaataaatatatggtraattagccaaggttagcagttcttt

SEQ ID NO: 629 - DNA, 51 bp, Homo sapiens
tattttaaaaccacatctaaaattgyatttattgtgtgcaacatgatgttt

SEQ ID NO: 630 - DNA, 51 bp, Homo sapiens
ccaattattcttaggtttagttgttsaacatcatctcagacttcttggaga

SEQ ID NO: 631 - DNA, 51 bp, Homo sapiens
ttcataaccatgatacagatttcaamcatataactcttttaaaaacacaaa

SEQ ID NO: 632 - DNA, 51 bp, Homo sapiens
gcttactggtttccactgttaaaatyaatggttggtgaaatatgtggtaga

SEQ ID NO: 633 - DNA, 51 bp, Homo sapiens
ttgatttctataatttggctttcaasatgacaatcagatctactattgtta

SEQ ID NO: 634 - DNA, 51 bp, Homo sapiens
agatcattagctacaagtcactatarcagatctaataaagcttgaaaaaac

SEQ ID NO: 635 - DNA, 51 bp, Homo sapiens
cacccaagtctcaatggtttaattaygtagaagatttattccttactcatg

SEQ ID NO: 636 - DNA, 51 bp, Homo sapiens
taatattttgtatgaattgtgacaayagaacataagtcttttattatgttt

SEQ ID NO: 637 - DNA, 51 bp, Homo sapiens
aatatatatatatatcgaaatttcartcctatatgagaatgtaaaaatgtt

SEQ ID NO: 638 - DNA, 51 bp, Homo sapiens
ttctggcagtctggacggaattaaayaactgtctacacaaatcaaagcttc

SEQ ID NO: 639 - DNA, 51 bp, Homo sapiens
gatatatataatgtataaagaataayttaggccaccaccagctttagaaat

SEQ ID NO: 640 - DNA, 51 bp, Homo sapiens
tagggtattctgatgctactgctccrgaactacactttgagtagcaaccct

SEQ ID NO: 641 - DNA, 51 bp, Homo sapiens
tctgcaggtctgttcgctcagcatcygatgagtgattctggctgtctgctg

SEQ ID NO: 642 - DNA, 51 bp, Homo sapiens
gctgatcactttccgttataggctamggtcattgtgataatgttggtcaaa

SEQ ID NO: 643 - DNA, 51 bp, Homo sapiens
atttcatcaaccagtgtgctggtcarctaccaagtcatattagatgctttt

SEQ ID NO: 644 - DNA, 51 bp, Homo sapiens
ctgattttactgtaataacatcaaaytttatattctaaccatttgcaaggg

SEQ ID NO: 645 - DNA, 51 bp, Homo sapiens
gcagctgccaaacagtagggcaacartatttccaaagccaagcaaggccaa

SEQ ID NO: 646 - DNA, 51 bp, Homo sapiens
ttgtgtacatcatgagaaatagaacygtatgatgtaagatttgtgctttat

SEQ ID NO: 647 - DNA, 51 bp, Homo sapiens
aggtgatcctcctgtctcagtgttayaaagtttccgtgccgcaaaagaaat

SEQ ID NO: 648 - DNA, 51 bp, Homo sapiens
gaggaagagtactatccaaggaaccsgctatcatcacaaattctgcagact

SEQ ID NO: 649 - DNA, 51 bp, Homo sapiens
tcttccaaatctcatggtctattacrgcttaggtgtccagagattcgtttg

SEQ ID NO: 650 - DNA, 51 bp, Homo sapiens
gagctgcaagttacaaacgactttakattgtacaaagaaaaatcggggagg

SEQ ID NO: 651 - DNA, 51 bp, Homo sapiens
caaatgccactgtgtatgcaaatcayctggaatcttgtgaaaatgtagatt

SEQ ID NO: 652 - DNA, 51 bp, Homo sapiens
ctacattaagtactaagaataccaamcatttggctgattgcagggagggag

SEQ ID NO: 653 - DNA, 51 bp, Homo sapiens
aagaaaacaattgagaaatataaaaraataggagcatttcataatgccaaa

SEQ ID NO: 654 - DNA, 51 bp, Homo sapiens
atcacaaaatcactcactcagtatcraactgaaaaatcagagagacgccta

SEQ ID NO: 655 - DNA, 51 bp, Homo sapiens
aactgaatccaaactcactattttcrcagtggttgttatcggtcctgccca

SEQ ID NO: 656 - DNA, 51 bp, Homo sapiens
atgcagtagcatctctgtggtaagaratttaatggaagtgtcattaaggaa

SEQ ID NO: 657 - DNA, 51 bp, Homo sapiens
tatataggaggagtccttaacctggracccatgagcttcttaaaatatatg

SEQ ID NO: 658 - DNA, 51 bp, Homo sapiens
gattaaaaccagcaaacctttttcarctcctggaaatcccagaaccttagg

SEQ ID NO: 659 - DNA, 51 bp, Homo sapiens
tagacagaagtctcaactcctagagraaatggatccagagacagtaaatat

SEQ ID NO: 660 - DNA, 51 bp, Homo sapiens
aatggtgaaagacttcataaattacyagttgaagaaaaactttctgagtgc

SEQ ID NO: 661 - DNA, 51 bp, Homo sapiens
ttctgtcacaaaggaatttggaatcragatagtttgggattgagccactcg

SEQ ID NO: 662 - DNA, 51 bp, Homo sapiens
tcaggatgtggtcaggtctccttacyggcaagtgtttgtagcaagatttga

SEQ ID NO: 663 - DNA, 51 bp, Homo sapiens
aaatatcaagaaaacacaatgaagarattaagagtagagaggttaggagcc

SEQ ID NO: 664 - DNA, 51 bp, Homo sapiens
aaagaagcttcatacccattagcggycactttccattccttcttccccaca

SEQ ID NO: 665 - DNA, 51 bp, Homo sapiens
tgatttctcctgtgcagcttcatagrcaccgcacaattttagagatatttg

SEQ ID NO: 666 - DNA, 51 bp, Homo sapiens
agagttatgcctcttagaattactayaatctataacgtctgagctctgttg

SEQ ID NO: 667 - DNA, 51 bp, Homo sapiens
caatcacaaaatatatgtgaatacastcttaattatgggccaggtactgag

SEQ ID NO: 668 - DNA, 51 bp, Homo sapiens
ctggtttttttaaatatttcaaggamctgacctgtaaaatgccactggaat

SEQ ID NO: 669 - DNA, 51 bp, Homo sapiens
taaaaagtttcactctattggtactrttaactgccaaccactttgaaggcc

SEQ ID NO: 670 - DNA, 51 bp, Homo sapiens
caaaatagctgatgcagagacctaartattcatagaacaaaaagtgaaaat

SEQ ID NO: 671 - DNA, 51 bp, Homo sapiens
ataattctacaggtggtgagagtgaygaaatagaatacaattttgttatga

SEQ ID NO: 672 - DNA, 51 bp, Homo sapiens
tttaaatcagtggactgagtaaaacmgattaccatccataacatgggtgag

SEQ ID NO: 673 - DNA, 51 bp, Homo sapiens
gtggggtcgtgtttgaatagcaaagrtgtgcgaacaaaccaaattaggaaa

SEQ ID NO: 674 - DNA, 51 bp, Homo sapiens
gttggaagaactagaatttgaaagaygctaagaagccacatgagacaccag

SEQ ID NO: 675 - DNA, 51 bp, Homo sapiens
taaatcttaaatttctcagtatcatyaaatttggaagagttagcaatgaag

SEQ ID NO: 676 - DNA, 51 bp, Homo sapiens
atgacaaactcctatttgttaatackctacaaaacatagtttgacatgctt

SEQ ID NO: 677 - DNA, 51 bp, Homo sapiens
acaagagtatctgaagagatttgaaytgtctattttgtaccacttaaatgt

SEQ ID NO: 678 - DNA, 51 bp, Homo sapiens
ctgtagtagacatcactattataaaytgatccccccagaggctgtaccaag

SEQ ID NO: 679 - DNA, 51 bp, Homo sapiens
ggtgaaactaaaagtgaaatgaagaytccatcaaccatcaaaagtcatccg

SEQ ID NO: 680 - DNA, 51 bp, Homo sapiens
taaggagtgaaaagtttaatagcaastaagaaggaagaaagaagagaacag

SEQ ID NO: 681 - DNA, 51 bp, Homo sapiens
atgacatcttcatcccgcaacacaayttcatcttgcaaacgtctcaggtct

SEQ ID NO: 682 - DNA, 51 bp, Homo sapiens
tgtttcatttaagaactggtctcaaytaagctcaaacagataactcttaac

SEQ ID NO: 683 - DNA, 51 bp, Homo sapiens
taaattagaacaacttttcactgacraagattaaactttttcatcattcct

SEQ ID NO: 684 - DNA, 51 bp, Homo sapiens
ctgattaccttcatctttaatattcrcacttattagatcaatctccctgta

SEQ ID NO: 685 - DNA, 51 bp, Homo sapiens
ctccacaccatttaaattgcctacakattcttgaaagtcagagaaaagagg

SEQ ID NO: 686 - DNA, 51 bp, Homo sapiens
ctgaggtttttagcctgagatgatakgtcaaaaccagggaggtgctcatgg

SEQ ID NO: 687 - DNA, 51 bp, Homo sapiens
gtggcttcttcaagtgacttctccayaaaacacccctgaggaaccttctgt

SEQ ID NO: 688 - DNA, 51 bp, Homo sapiens
atggggattttcccaatgctagcgaratgtgtcaaatggttcaacctcaac

SEQ ID NO: 689 - DNA, 51 bp, Homo sapiens
cttgatctacatttcatcctctctcrctttctgttttttcttgaccctgtt

SEQ ID NO: 690 - DNA, 51 bp, Homo sapiens
ctccaagacattagaatattttcaartaactccatggtgttccagaacaaa

SEQ ID NO: 691 - DNA, 51 bp, Homo sapiens
tcctctcctgtttaactagtttgtamctctctctagattagattattccca

SEQ ID NO: 692 - DNA, 51 bp, Homo sapiens
ccatttccattgtgtatattcaccaygtctccagtatccattcctccatgg

SEQ ID NO: 693 - DNA, 51 bp, Homo sapiens
agcttaaatgagtcactcccttgtawacatcctaaattctcttgctcctcc

SEQ ID NO: 694 - DNA, 51 bp, Homo sapiens
taacttttttttttagtatctgtacragtcataattttacctttttgaaaa

SEQ ID NO: 695 - DNA, 51 bp, Homo sapiens
tttttaaaaaataataggaaggagargtaaaattttagacttagaaaaacc

SEQ ID NO: 696 - DNA, 51 bp, Homo sapiens
atcacaacacatcctgaaagctaccrgaaggaggatacagtaatggagtcc

SEQ ID NO: 697 - DNA, 51 bp, Homo sapiens
caactgtgccaatagtaaagggctaycttgaagccaggtatgttcaacata

SEQ ID NO: 698 - DNA, 51 bp, Homo sapiens
cccttttctttgtcaccgtgtgtacrgtaaagaaccaggcaacatggtgcc

SEQ ID NO: 699 - DNA, 51 bp, Homo sapiens
agctaaaaatatatttagaaagccawatctgaactgaatttttgatgcacg

SEQ ID NO: 700 - DNA, 51 bp, Homo sapiens
gatgcttgtattctctataacattargaaatattaaaatgttgctatacat

SEQ ID NO: 701 - DNA, 51 bp, Homo sapiens
cacagccaaaccgtatcagtaataaragaatgaaagtgggagggaatgatg

SEQ ID NO: 702 - DNA, 51 bp, Homo sapiens
ccagatccagctgaaagatacagctraaagagacagcctgcagcatcatca

SEQ ID NO: 703 - DNA, 51 bp, Homo sapiens
catgaagtacaaaaaactccacatasattaaatataaagacatcttcactg

SEQ ID NO: 704 - DNA, 51 bp, Homo sapiens
gggactttattttgtgtgggagagarcagaatatggatagatttgctgaag

SEQ ID NO: 705 - DNA, 51 bp, Homo sapiens
acaagtgctatctcatttaattatcycaattctgtaaagtagatattatta

SEQ ID NO: 706 - DNA, 51 bp, Homo sapiens
attcatttgcttcttgtatcaactaycatggctttcctccttccttacttt

SEQ ID NO: 707 - DNA, 51 bp, Homo sapiens
attttcagaaagatgtagtggtgaayagaagcgaacactacaaaaagatga

SEQ ID NO: 708 - DNA, 51 bp, Homo sapiens
acacaatccaagcaaaataaaaatcrcaatatagcaatatgggagctgagg

SEQ ID NO: 709 - DNA, 51 bp, Homo sapiens
tagatattgtatggcatattctcacrcttcattcactggccacctgatgtg

SEQ ID NO: 710 - DNA, 51 bp, Homo sapiens
gtaagttttcttatagaaaaaggatraaagtattgtagaagtcattcctat

SEQ ID NO: 711 - DNA, 51 bp, Homo sapiens
tcttacccctaacttcctctttcaayttaccatgtaattcctagtttatct

SEQ ID NO: 712 - DNA, 51 bp, Homo sapiens
atagtgggaagaaacaactgtctcarcttttcttcctacttcagtgctatg

SEQ ID NO: 713 - DNA, 51 bp, Homo sapiens
aagacatggagaaatcttaaacacaytctaagtgaaagaagccaatctgaa

SEQ ID NO: 714 - DNA, 51 bp, Homo sapiens
tatatatgcacccaacactagagcayctggattcataaaacaaattcttct

SEQ ID NO: 715 - DNA, 51 bp, Homo sapiens
tggtttgtggttcatcttaggctaayatacgcctttatgtgtacctcttta

SEQ ID NO: 716 - DNA, 51 bp, Homo sapiens
atgatctggacactaccacagaagamggagtgatagagagacagaaaatgg

SEQ ID NO: 717 - DNA, 51 bp, Homo sapiens
gtttcttactgccttggttgcaggamctctagctaaacacattggggcatc

SEQ ID NO: 718 - DNA, 51 bp, Homo sapiens
accatattgggatgctatataaaaaytcaaaacccagacaagactgggaag

SEQ ID NO: 719 - DNA, 51 bp, Homo sapiens
acagagcaaacaagcaaaaggaatcmgaaagaagtctgaacatactttaga

SEQ ID NO: 720 - DNA, 51 bp, Homo sapiens
gcttcactggccttctcgcttttctyggacacaccacactctttcttgtct

SEQ ID NO: 721 - DNA, 51 bp, Homo sapiens
caaggaactggtcagtgtagggaaascacacattttcttgaccagaagtaa

SEQ ID NO: 722 - DNA, 51 bp, Homo sapiens
cattcattctccaattcctaaaatayggcttctgtctgggttacttctctg

SEQ ID NO: 723 - DNA, 51 bp, Homo sapiens
ggaagatccagccaatgacttgatargacaataggaagatttcagagtcct

SEQ ID NO: 724 - DNA, 51 bp, Homo sapiens
ctctctactaccactgcttagcatartaattggcatttgttattactatat

SEQ ID NO: 725 - DNA, 51 bp, Homo sapiens
aattagaccacaggagagtattagasataggacaactgctgacttgtaaaa

SEQ ID NO: 726 - DNA, 51 bp, Homo sapiens
ctgtaagtgaatgaattttgtgatgkattatattattatgccaaatattca

SEQ ID NO: 727 - DNA, 51 bp, Homo sapiens
gaacttgttagaaaagcagattatgsaaccctgtctcagacctactgagtg

SEQ ID NO: 728 - DNA, 51 bp, Homo sapiens
taaatctaacaaaaactgtgtaagarataaatgtttaaaatcctaaaatga

SEQ ID NO: 729 - DNA, 51 bp, Homo sapiens
aatcaccctgactgtagagtagatartagatttctaacaggcaagatagtc

SEQ ID NO: 730 - DNA, 51 bp, Homo sapiens
tatgagccccactgtggatctgtacrtttccttagggcattggagcttcag

SEQ ID NO: 731 - DNA, 33 bp, Homo sapiens
tttctctttcatttgcrttcgtatcaataggct

SEQ ID NO: 732 - DNA, 51 bp, Homo sapiens
atatatggcatcttgataggccaagracaatgtataatttctattgctttc

SEQ ID NO: 733 - DNA, 51 bp, Homo sapiens
aagggctgaggtatttgattaaatgrccacatgaaaaatggagatgtagaa

SEQ ID NO: 734 - DNA, 51 bp, Homo sapiens
acaagacctaacttgggtgaatgttkaacaagtccagggaaatccagatgc

SEQ ID NO: 735 - DNA, 51 bp, Homo sapiens
cttatgggtcagatctggaaagttayaaaaaggtgcaaaccatcagggttt

SEQ ID NO: 736 - DNA, 51 bp, Homo sapiens
ggtttcctctcttggcaaacatagargctcaactgtgttctctgaggccct

SEQ ID NO: 737 - DNA, 51 bp, Homo sapiens
ttttcatttttatcttatagaaccaraattacatagcttacatttccttta

SEQ ID NO: 738 - DNA, 51 bp, Homo sapiens
ctagccccatggtttcaaatacaacsagcaatgaaatattttcacagccta

SEQ ID NO: 739 - DNA, 51 bp, Homo sapiens
ttctgtatgtgtgttgtgaattataratctggcttttcctacaaatgtggc

SEQ ID NO: 740 - DNA, 51 bp, Homo sapiens
aacaggatcaggtgttattactaccragaagaagtgtgattcttgaatctt

SEQ ID NO: 741 - DNA, 51 bp, Homo sapiens
gaattaattaagttaaaattacctaygatatgctctatagattacattctt

SEQ ID NO: 742 - DNA, 51 bp, Homo sapiens
gtccttttttgaagtaagggacaagragtaagtattttcttttttaagttt

SEQ ID NO: 743 - DNA, 51 bp, Homo sapiens
tatgcagaattgtggatactgtaaasagagcagtgagagacaataagacag

SEQ ID NO: 744 - DNA, 51 bp, Homo sapiens
gcatacaattcctgatcagagcccaygttccttacctaaaagaaggtggaa

SEQ ID NO: 745 - DNA, 51 bp, Homo sapiens
ggtgagcagagagcaaagaataaaartagaaggaggagaaaagagacagag

SEQ ID NO: 746 - DNA, 51 bp, Homo sapiens
gataaagaagagttagaaaatcacaygtgttgtaaatgctcatttgtttaa

SEQ ID NO: 747 - DNA, 51 bp, Homo sapiens
ttaaaagggtgatttggagtaaaaaygaatagtacagacctcaaaaaccgt

SEQ ID NO: 748 - DNA, 51 bp, Homo sapiens
ctggtccagatccactaattctttcyctgtacctcacagcaccagacctct

SEQ ID NO: 749 - DNA, 51 bp, Homo sapiens
agcccagttttttagtagctcaagaraagatcttcaatttatgtctccgcc

SEQ ID NO: 750 - DNA, 51 bp, Homo sapiens
cataatattgatgtactacagtgacyattaaagcatttaccttttctggaa

SEQ ID NO: 751 - DNA, 51 bp, Homo sapiens
aaaggctttttgctttaaaaagcagyctcacaggtgtttgagaggcaaggg

SEQ ID NO: 752 - DNA, 51 bp, Homo sapiens
tcatcatcatctgtgtactttctcayatcccaccgttgtactccaaaatca

SEQ ID NO: 753 - DNA, 51 bp, Homo sapiens
tagtcagctatgtaaattaaaatccwcattatgtataagcatagagaggta

SEQ ID NO: 754 - DNA, 51 bp, Homo sapiens
caatctcatcagatctttataaacarctctgtaggatgaatattttatttc

SEQ ID NO: 755 - DNA, 33 bp, Homo sapiens
tttaacttatctttgcrgtatttataagattgt

SEQ ID NO: 756 - DNA, 51 bp, Homo sapiens
aatggtacatatggaatacttagaaracttaattactttcctctacataac

SEQ ID NO: 757 - DNA, 51 bp, Homo sapiens
tcgattttctgcaatgacaacaacgyagaaaacagggtgaacttcctggaa

SEQ ID NO: 758 - DNA, 51 bp, Homo sapiens
aagatcaggaattggcttggaaagcragagcgtagtgaatagtcgcttata

SEQ ID NO: 759 - DNA, 51 bp, Homo sapiens
atgtgttgtgagtcttgtgctaataytagaaaaggtctgggctagggctgt

SEQ ID NO: 760 - DNA, 51 bp, Homo sapiens
aaactccaccacttacaacagcatcwaaaaaacattaaatgtctagaaata

SEQ ID NO: 761 - DNA, 51 bp, Homo sapiens
tatgtatagatttaacattattccartaaaaatctcagagggattattgat

SEQ ID NO: 762 - DNA, 51 bp, Homo sapiens
gaagatttggcttttctcctgaagtraaagaatacatgaatctgatgagac

SEQ ID NO: 763 - DNA, 51 bp, Homo sapiens
acatatagatttactattgctatatstctttcctcattgacccattcatca

SEQ ID NO: 764 - DNA, 51 bp, Homo sapiens
ctttactgacaatgccaatagttgcratcttaggggggccatttattgagt

SEQ ID NO: 765 - DNA, 51 bp, Homo sapiens
ctcactgaaagaggagagatttaagrccattcagcagcatcttattttatt

SEQ ID NO: 766 - DNA, 51 bp, Homo sapiens
gtttcattatactgtggttgaataasgtactcggcatgacttcaatcttca

SEQ ID NO: 767 - DNA, 51 bp, Homo sapiens
ggttttatgtatgacaatgtattcaygaagcagtagaaaatccatcttggt

SEQ ID NO: 768 - DNA, 51 bp, Homo sapiens
cagctgctgggaaagaccatttaaarcatttaatatcctccctacctctct

SEQ ID NO: 769 - DNA, 51 bp, Homo sapiens
taaaggaaacaagacatataataccmagtagaaagaaacatgaaattctat

SEQ ID NO: 770 - DNA, 51 bp, Homo sapiens
tttttcttaactgcttttaaaaatgyatctctttacctttggttttaagcc

SEQ ID NO: 771 - DNA, 51 bp, Homo sapiens
aagaaaagaacttcttgaaggcatawcaatggaaactatccaaatttagca

SEQ ID NO: 772 - DNA, 51 bp, Homo sapiens
gcatgcacttgcctaagtactttaaytgtagctttcaagccttctggactg

SEQ ID NO: 773 - DNA, 51 bp, Homo sapiens
attcagcttcctcgccagtctggagycctttgaagacagatcactcctgtc

SEQ ID NO: 774 - DNA, 51 bp, Homo sapiens
taggaccacgtggatttctcatcaarcttactggagaaagtcccgttgcta

SEQ ID NO: 775 - DNA, 33 bp, Homo sapiens
ttctttcaaaattcgartcacttctctcaaact

SEQ ID NO: 776 - DNA, 51 bp, Homo sapiens
aaactgtccagaccatcaagagtacrcatattcaaaatatcattgtgaatc

SEQ ID NO: 777 - DNA, 51 bp, Homo sapiens
atgcactttgagacttcttagttacraacaaccattccagggagtttaaat

SEQ ID NO: 778 - DNA, 51 bp, Homo sapiens
gttttgatatatgtatacattgtgayatgattactgcaatcaggttagtta

SEQ ID NO: 779 - DNA, 51 bp, Homo sapiens
gggtgccccatgtgaagccctcgaargatgacatgagggccctgagaccct

SEQ ID NO: 780 - DNA, 51 bp, Homo sapiens
ctgttatctaaaaacatatggataawaccaacatgactagaaaccaaacga

SEQ ID NO: 781 - DNA, 51 bp, Homo sapiens
cagatctgctgtgtatgcatcagccraattatcctgtctcactaacctaag

SEQ ID NO: 782 - DNA, 51 bp, Homo sapiens
cccctgttcaagtgtacattttagcyactctaatttttctgacaattgttt

SEQ ID NO: 783 - DNA, 51 bp, Homo sapiens
aatcaatctaaccatccaccatgtgyacatcctgtttattattcaatgcac

SEQ ID NO: 784 - DNA, 51 bp, Homo sapiens
tagcaaataacatatcttgaagaacrctaattttaccactaaaatatttct

SEQ ID NO: 785 - DNA, 51 bp, Homo sapiens
tcgttgacctccattttgcactttasataaacccagttttgatgctggtgt

SEQ ID NO: 786 - DNA, 51 bp, Homo sapiens
gaaaacagataaattctaggttataraaggccacggaggactatggaagct

SEQ ID NO: 787 - DNA, 51 bp, Homo sapiens
gataattctgaagttttgttttctartacttccttgaattcagcatgtact

SEQ ID NO: 788 - DNA, 51 bp, Homo sapiens
actttagccatagggaaactttgatyaaactaataatacataaggaagttg

SEQ ID NO: 789 - DNA, 51 bp, Homo sapiens
cttagggaaaatgagactatctctcygaccttcttatatcctcctactttc

SEQ ID NO: 790 - DNA, 51 bp, Homo sapiens
aaactaagtaatcatcacaccagctragaaccataagttccttgacccagg

SEQ ID NO: 791 - DNA, 51 bp, Homo sapiens
ttcaaaagtttcaggaaactgcttgmattagggtgaatctgatctaatttt

SEQ ID NO: 792 - DNA, 51 bp, Homo sapiens
ctgtgtagaacactttgtagaagaayactttgtatgtgttatctaaattgg

SEQ ID NO: 793 - DNA, 51 bp, Homo sapiens
tttaattataatctctgctctttaaygtcaatatccaatgctttgcttttc

SEQ ID NO: 794 - DNA, 51 bp, Homo sapiens
cagataattttgtcttattatctcamagattacttgtttcagccatacacc

SEQ ID NO: 795 - DNA, 51 bp, Homo sapiens
ggagaaatagattgcaatacaacaayagtagaggacttcaataccccaatt

SEQ ID NO: 796 - DNA, 51 bp, Homo sapiens
tgaaagagggctctggggaagtaaayggaactgcagcaagtaaattaataa

SEQ ID NO: 797 - DNA, 51 bp, Homo sapiens
caagatagccagaaattatatttcckgatggagtatacaatctgcctttgg

SEQ ID NO: 798 - DNA, 51 bp, Homo sapiens
atctactaccctttctagtgctttayctatgtcatcacaaagattccagaa

SEQ ID NO: 799 - DNA, 51 bp, Homo sapiens
ctttgtttaaatataacaataatccyattaccacacacacaatccagaagt

SEQ ID NO: 800 - DNA, 51 bp, Homo sapiens
ttatcaggtgttaacattagagtgayacctggtaaaaattgctgtaattgc

SEQ ID NO: 801 - DNA, 51 bp, Homo sapiens
taatgcaagcatgctacatacactayaatttttgtaaccttatttgatcat

SEQ ID NO: 802 - DNA, 33 bp, Homo sapiens
ataatacttcctgctawtgttgcagctgtcact

SEQ ID NO: 803 - DNA, 51 bp, Homo sapiens
cttctcatcctgaattgccatttcartagtgttatctctagtctgctggat

SEQ ID NO: 804 - DNA, 33 bp, Homo sapiens
gcaaaaaagataatagrtgcggccaaatttgag

SEQ ID NO: 805 - DNA, 51 bp, Homo sapiens
gctagatgttaatgtcatcataagcratttttaaaaattatcctaactcta

SEQ ID NO: 806 - DNA, 51 bp, Homo sapiens
cttttgaaaggctcaggttgccttakagtcttccataaaaatactgagggc

SEQ ID NO: 807 - DNA, 51 bp, Homo sapiens
cagggctcagtgacttctgtattccyttgcagatcctgagctcccagagtg

SEQ ID NO: 808 - DNA, 51 bp, Homo sapiens
gtgttctaatctgacacaaattgtamtaatctattcttagagaaatttgta

SEQ ID NO: 809 - DNA, 51 bp, Homo sapiens
caaagcaagaatatgcaaaatggcasataccgcatgaccttgatagttaga

SEQ ID NO: 810 - DNA, 51 bp, Homo sapiens
ttatgaaattccaattccactgtgaratattcccgaactcattttatcagc

SEQ ID NO: 811 - DNA, 51 bp, Homo sapiens
ctgaataaaagcaacacttgaatgasaccagaaatgtaacatttccaagtg

SEQ ID NO: 812 - DNA, 51 bp, Homo sapiens
ggggatagctgtgatatagcactggscaaagcatgcttcagagatgttttg

SEQ ID NO: 813 - DNA, 51 bp, Homo sapiens
caatatttcctttgcatgtcctcaarctataaccaaattcggggagaggag

SEQ ID NO: 814 - DNA, 51 bp, Homo sapiens
ctctttgctatggaatcgggctccaracaaacttatctgtaaagtagcagc

SEQ ID NO: 815 - DNA, 51 bp, Homo sapiens
agcacaagtgacttgctgggtaagcyagaataagactggaagctgagggtc

SEQ ID NO: 816 - DNA, 51 bp, Homo sapiens
tttcctttcttggatttaaaagcatsactaattctttttctactttaaact

SEQ ID NO: 817 - DNA, 51 bp, Homo sapiens
aaaggaactgaaggaaaagagagcayggatatatgagtgtggttgcatagg

SEQ ID NO: 818 - DNA, 51 bp, Homo sapiens
tttcagcttgtgttggaaacgtgatyaacttagagaaagaagctgcagaag

SEQ ID NO: 819 - DNA, 51 bp, Homo sapiens
atctaagattacaaactcacttatcratggctacaattattcttttttcct

SEQ ID NO: 820 - DNA, 51 bp, Homo sapiens
cttcaagataaacatggataaaatayattcgttcagtcaacaaatgcttag

SEQ ID NO: 821 - DNA, 51 bp, Homo sapiens
atgtagggtcatcctaagattttcarggtaagccttaaggttaatctattg

SEQ ID NO: 822 - DNA, 51 bp, Homo sapiens
tttttgtaaaatttttgctctttaakgattataatcttttgcatagctgga

SEQ ID NO: 823 - DNA, 51 bp, Homo sapiens
gcctaggtagactagttttaagagamcatagaactaaaaaaatgtggaaaa

SEQ ID NO: 824 - DNA, 51 bp, Homo sapiens
aactattccttcatagagaaactaaygtgtatatttttaataacaggttta

SEQ ID NO: 825 - DNA, 51 bp, Homo sapiens
cacacccagcctcaacctgtcattawcaagaattttggtgcaaatatacca

SEQ ID NO: 826 - DNA, 51 bp, Homo sapiens
ggatcacagcttttatttaattacaygggccaagtgttctgggcattctct

SEQ ID NO: 827 - DNA, 51 bp, Homo sapiens
gttacagaacaatatcagaagcctaygtgccatctaaaattttcttttatt

SEQ ID NO: 828 - DNA, 51 bp, Homo sapiens
ctctaagggtgagtgtgggaggacartaggaggtacaaacacaagtaaatg

SEQ ID NO: 829 - DNA, 51 bp, Homo sapiens
aatacctgttagtgcattattattaycttgaacactttcctctgacttaga

SEQ ID NO: 830 - DNA, 51 bp, Homo sapiens
gagtattttgtgaaaagcaggccacrattttttatgtctgtgatccatcct

SEQ ID NO: 831 - DNA, 51 bp, Homo sapiens
tttcatggtgttttctcaattaacartgtacttagtgctttaattttgtgg

SEQ ID NO: 832 - DNA, 51 bp, Homo sapiens
ctaggagaaagcaggtggaagaataygaaaggactagacttgctgagtctt

SEQ ID NO: 833 - DNA, 51 bp, Homo sapiens
tgtcaactgagtatacactggagacygaaaccatgatgaagtcagttaaaa

SEQ ID NO: 834 - DNA, 51 bp, Homo sapiens
ggaacataaaaatatcagcagtgcakatgaagagacaaagaaggtcataaa

SEQ ID NO: 835 - DNA, 51 bp, Homo sapiens
tggaggattttgaacaggagtgtacyatgttctaatttagttttgaaatga

SEQ ID NO: 836 - DNA, 51 bp, Homo sapiens
gtcctgccaaaagtcacaagttgaarttgaataaccactgtaatagtatta

SEQ ID NO: 837 - DNA, 33 bp, Homo sapiens
gtattagaagcagatgytcttataaactaaatg

SEQ ID NO: 838 - DNA, 51 bp, Homo sapiens
gaataaaaatgtgtgtttcataagcrataaatgatttgtagttatcctggt

SEQ ID NO: 839 - DNA, 51 bp, Homo sapiens
tgaaaaatttatagtcactgatattraagactgaacaaataaattcaatgg

SEQ ID NO: 840 - DNA, 51 bp, Homo sapiens
caatgtataggagcaagtgtatgaarttctgaaggaagggctttctaggta

SEQ ID NO: 841 - DNA, 51 bp, Homo sapiens
gtgcatggtgcagaggtggtgttctrtaaatattcattccctttccgcatc

SEQ ID NO: 842 - DNA, 51 bp, Homo sapiens
cacagccgtaccagtgacttgaatargatagaggaccatcaagctgaggga

SEQ ID NO: 843 - DNA, 51 bp, Homo sapiens
atagagaaatatctttatgaccttgmgattgccagtgagttcttcaacagg

SEQ ID NO: 844 - DNA, 51 bp, Homo sapiens
tcagtttattagcaaccataataccmccttggatataacatagcatactca

SEQ ID NO: 845 - DNA, 51 bp, Homo sapiens
ttccagccaatgaatggaaagacaaygtgttatatcctaccagcctacttt

SEQ ID NO: 846 - DNA, 51 bp, Homo sapiens
tgcttttattgtggtgtgatagcagyctgcatatacattttctaactacac

SEQ ID NO: 847 - DNA, 51 bp, Homo sapiens
aggcatatacccaatagcaaataaaygaaaattgatgcaatgctacaaaaa

SEQ ID NO: 848 - DNA, 51 bp, Homo sapiens
tattgcatactttgggtcacttatamcttctcacaccaatacaaaaacatg

SEQ ID NO: 849 - DNA, 51 bp, Homo sapiens
aggcaatcatgataataataattccmatcataactaccttttatcaagtgc

SEQ ID NO: 850 - DNA, 51 bp, Homo sapiens
ttgaaataatcgaaatgggaaaaacmgaacttatccataacgcctccccac

SEQ ID NO: 851 - DNA, 51 bp, Homo sapiens
ctacccgctaccaggctcactctcayggtggttctggggatgatgtgcact

SEQ ID NO: 852 - DNA, 51 bp, Homo sapiens
ttattattatttagaaatttgcactyaccattaagaatgatcaaaatatag

SEQ ID NO: 853 - DNA, 51 bp, Homo sapiens
acattcacatactctatggcatccaraatcttgaaggacacatagcaggtc

SEQ ID NO: 854 - DNA, 51 bp, Homo sapiens
ccctagtaggaaagtaaccttgaaaygaccaatgcactctttttttttttt

SEQ ID NO: 855 - DNA, 51 bp, Homo sapiens
ccatgtctgataccttcagtgttacrgaaactactacgtctcagacaaagc

SEQ ID NO: 856 - DNA, 51 bp, Homo sapiens
aatctttacaatacccttaattccaygtgctatttccagttttctgagagt

SEQ ID NO: 857 - DNA, 51 bp, Homo sapiens
gagcaattaagcttgagggaaaatgsaaactatacatacaaccacagtgac

SEQ ID NO: 858 - DNA, 51 bp, Homo sapiens
ttatatcattttagcttcaattccayaaattagatctaaacattctataca

SEQ ID NO: 859 - DNA, 51 bp, Homo sapiens
caatataatctacttccctgttataygttagactttcttccctgggattac

SEQ ID NO: 860 - DNA, 51 bp, Homo sapiens
ggaatctttgattagagctcatcaawggttcagagggacctttggtgctct

SEQ ID NO: 861 - DNA, 51 bp, Homo sapiens
ttctcccacttgttcttcattcctayatttattcaacttcccttataatcc

SEQ ID NO: 862 - DNA, 51 bp, Homo sapiens
cttgaaaacatattaattgtttaccratctttctcctaaaaataaagcttc

SEQ ID NO: 863 - DNA, 51 bp, Homo sapiens
ttgttcctttttagaaaccacttaaragaaagattaaatgttactctgatg

SEQ ID NO: 864 - DNA, 51 bp, Homo sapiens
cttggtttcataataccgtactgccratttataaactatattttagtctac

SEQ ID NO: 865 - DNA, 51 bp, Homo sapiens
tgtgtattgcagtacctagtagtcartaaatccatgttgaatgaatgaatg

SEQ ID NO: 866 - DNA, 51 bp, Homo sapiens
atctgattgttgatcaaagaaggcaytgatgtttattttaagtagtgctaa

SEQ ID NO: 867 - DNA, 51 bp, Homo sapiens
tactttctggcatttcttctaaatcrctaagcctctcttcagcttccagta

SEQ ID NO: 868 - DNA, 51 bp, Homo sapiens
tatttagtgcttactacataaagagmactgggctagaagcagttgagagag

SEQ ID NO: 869 - DNA, 51 bp, Homo sapiens
tacaacttatttttttttaactggckagtaccagaagatttacaaatgtaa

SEQ ID NO: 870 - DNA, 51 bp, Homo sapiens
tagagtcaattgactgtaggtgatamacatatacacaaaataccttcacag

SEQ ID NO: 871 - DNA, 51 bp, Homo sapiens
cagatccaccattatacatttaaatyatccagtctgctatgtggcttttcc

SEQ ID NO: 872 - DNA, 51 bp, Homo sapiens
ggtaactggaaacctagccaagctcratgatgaacgcagccatgtgagtga

SEQ ID NO: 873 - DNA, 51 bp, Homo sapiens
atcctggagtaagcaacttggttaayggtagaaccacttaattttttttct

SEQ ID NO: 874 - DNA, 33 bp, Homo sapiens
atcagaacttggaatamtagcatactgagagcc

SEQ ID NO: 875 - DNA, 51 bp, Homo sapiens
ttcaattcttctcaaagtgtcactcragtatttctgagcattcttaggggt

SEQ ID NO: 876 - DNA, 51 bp, Homo sapiens
acacacatctcctaatgcttacaccratgtcggtttccatgtcttcgtccc

SEQ ID NO: 877 - DNA, 51 bp, Homo sapiens
ataaagttaaattttaacaaaaaatkaatcagttctactcttacttgcttt

SEQ ID NO: 878 - DNA, 51 bp, Homo sapiens
taaaagcttcctttgctttatttaaratcacagcagttacctcacattgcc

SEQ ID NO: 879 - DNA, 51 bp, Homo sapiens
aaaatagttgctgtgttttctcaacrgtcgtcagaattataatgctattct

SEQ ID NO: 880 - DNA, 51 bp, Homo sapiens
acagcactgttccacctctcttccaytgaattttacatatgaacagactat

SEQ ID NO: 881 - DNA, 51 bp, Homo sapiens
cagaaataaaaacaaacaatctttakgaagctaaactaaggtataaccatt

SEQ ID NO: 882 - DNA, 51 bp, Homo sapiens
atgtgtaatatacttggcacagcgaraaaatggatttaaaagacaaaaatg

SEQ ID NO: 883 - DNA, 51 bp, Homo sapiens
aacactgttttcctctggattaaaarccattccccatcttcctatccttca

SEQ ID NO: 884 - DNA, 51 bp, Homo sapiens
tgggctgttttcagaagtatctttayagcatttgttaattttgttgtaact

SEQ ID NO: 885 - DNA, 51 bp, Homo sapiens
gtttcttatacaactcagagctttaygttatttagactgtctgaacactgg

SEQ ID NO: 886 - DNA, 51 bp, Homo sapiens
caagtcttgtctaagatgtctgaacyattaaatttaccattttgtttttct

SEQ ID NO: 887 - DNA, 51 bp, Homo sapiens
aacattgtgtcccaagaacttcattwaaaaagctgggtagtggtgcccaga

SEQ ID NO: 888 - DNA, 51 bp, Homo sapiens
gactttagaaaccctctctccaaaaytgattcaagcacagcctcatatgga

SEQ ID NO: 889 - DNA, 51 bp, Homo sapiens
acatcacttaattctcacagtagaarataatacagaacgaaaagccatgtt

SEQ ID NO: 890 - DNA, 51 bp, Homo sapiens
ctcctgcgttgctttgataactaaayaaaatggttctatctgaatagttct

SEQ ID NO: 891 - DNA, 51 bp, Homo sapiens
ggctgataatttttggccttagttcyaggtagattaaaaagctgctagctc

SEQ ID NO: 892 - DNA, 51 bp, Homo sapiens
atttcaatactgtatggagatagaartagtctaatttgcttatcactgcca

SEQ ID NO: 893 - DNA, 51 bp, Homo sapiens
gatctctcagcatacctgtgtggacragataaagaaatatggctagatgca

SEQ ID NO: 894 - DNA, 51 bp, Homo sapiens
tggaattggaatttcataaacacaasacaatgaaagaacacctatgtgatt

SEQ ID NO: 895 - DNA, 51 bp, Homo sapiens
atgctgctatgattattattattgcwaaattaaatcgtacttagaagttct

SEQ ID NO: 896 - DNA, 51 bp, Homo sapiens
gaatccccattagactatcagtgaayaccttagcagaaaccttgaaagtca

SEQ ID NO: 897 - DNA, 51 bp, Homo sapiens
tgttggattcattaccttcaatatcrccctctttcttctgtcctatcctgg

SEQ ID NO: 898 - DNA, 51 bp, Homo sapiens
gaacagacatgtgcttctgtacagcrttatttatcaacaagtaaagctgtg

SEQ ID NO: 899 - DNA, 51 bp, Homo sapiens
gtctgactcacaggttcctgattgcsaagatgtgttacaggaaaactgtgc

SEQ ID NO: 900 - DNA, 51 bp, Homo sapiens
aaaaacttttattttaaagcatcccrccataggctgaggaacactggtagg

SEQ ID NO: 901 - DNA, 51 bp, Homo sapiens
actctgagaaagatacctaacaaggmacatgatagaaatctggttataact

SEQ ID NO: 902 - DNA, 51 bp, Homo sapiens
atatctatatgaaagattcatattawcagatgggtttatttcttttttttt

SEQ ID NO: 903 - DNA, 51 bp, Homo sapiens
cagactttggtaatagtcctgtaatmaactcttctcatgcaacagtgtagg

SEQ ID NO: 904 - DNA, 51 bp, Homo sapiens
aacatttacattaacagggatttcawagaagttgattctggcccttatgat

SEQ ID NO: 905 - DNA, 51 bp, Homo sapiens
cttgacagatctgtaactgaccttamaactttattgcaccagctaaatcca

SEQ ID NO: 906 - DNA, 51 bp, Homo sapiens
agttgcagtttcaatggtagatgaarttcaaaggtcaaagtacctaaaaat

SEQ ID NO: 907 - DNA, 51 bp, Homo sapiens
gaaaaggcaagatagatgcttatttrtgaccttttaattcatcattttact

SEQ ID NO: 908 - DNA, 51 bp, Homo sapiens
agatctgaaactctacccattaaacracttcccatcttcccatccccacag

SEQ ID NO: 909 - DNA, 51 bp, Homo sapiens
ctttgccttcctgaccgttagatacrattaatttggggtttgttttttctg

SEQ ID NO: 910 - DNA, 51 bp, Homo sapiens
ctgtatcagtgtgcctatctaagtgmaaactcagggagggcaggatggaac

SEQ ID NO: 911 - DNA, 51 bp, Homo sapiens
ctcaggatagtagtaaaccttaagayagttcgtaatcaaggcccccaagtc

SEQ ID NO: 912 - DNA, 51 bp, Homo sapiens
ctccatttttttgctgctctttcatwaaatgtgaggcagcacagtgcagag

SEQ ID NO: 913 - DNA, 51 bp, Homo sapiens
cagtcaaaacctttatatcaatacaygagaaacaagtaaactatttccctg

SEQ ID NO: 914 - DNA, 51 bp, Homo sapiens
catttatacatagaggaagggcttayggactgagaaagggagaacatggta

SEQ ID NO: 915 - DNA, 51 bp, Homo sapiens
aggcaacctacactagctgcctatayaagttaacctactcttcttgtcaga

SEQ ID NO: 916 - DNA, 51 bp, Homo sapiens
aatacaggagggttctaagatgaagscttattgtctaggtcacacaattga

SEQ ID NO: 917 - DNA, 51 bp, Homo sapiens
tctttctcaatactgggcctcagtaycaaactattaacgcaagatctcaag

SEQ ID NO: 918 - DNA, 51 bp, Homo sapiens
agtcccttcatagtgacttgctctcyagttctcgggacgctgcaggccttg

SEQ ID NO: 919 - DNA, 33 bp, Homo sapiens
cagttaatcttttgcamcatgaattggaattta

SEQ ID NO: 920 - DNA, 33 bp, Homo sapiens
tccccatgtctatgccktggttcctgctatcta

SEQ ID NO: 921 - DNA, 51 bp, Homo sapiens
gggagtgtgttccaagtggagtagayagcaggaaccaaggcatagacatgg

SEQ ID NO: 922 - DNA, 51 bp, Homo sapiens
tagtgtttatgtccacatcacatggratattttatttttcaggcctcttca

SEQ ID NO: 923 - DNA, 51 bp, Homo sapiens
cctaaatatgctctgcaaggaatttkcccaacaaaggcacttctctagtta

SEQ ID NO: 924 - DNA, 51 bp, Homo sapiens
gtccaacccctcactttacagagaamgaatcttaggaaaagagaaatgact

SEQ ID NO: 925 - DNA, 51 bp, Homo sapiens
ggttaagttaggggccagtcagtgtraaaacaagtctcccatctaaatccg

SEQ ID NO: 926 - DNA, 51 bp, Homo sapiens
gttatttgtttttgctggttgcgaakcactgggatattttctttctttttc

SEQ ID NO: 927 - DNA, 51 bp, Homo sapiens
ataaggtagatgttaccactacagargattttcttatctaatgcatttgtt

SEQ ID NO: 928 - DNA, 51 bp, Homo sapiens
acttttaacggttcctggaattttayaactgcataggcataagaacatttt

SEQ ID NO: 929 - DNA, 51 bp, Homo sapiens
atgcatagtgtcgtcaaatgattgcmcaatagttaagtgtaacaatatgaa

SEQ ID NO: 930 - DNA, 51 bp, Homo sapiens
gcacaaactcctaatcaaatcataaycctctgcataactcatggtcaaatc

SEQ ID NO: 931 - DNA, 51 bp, Homo sapiens
aggaaattttaacactactactaatwagcatttactaagcatgttggccat

SEQ ID NO: 932 - DNA, 51 bp, Homo sapiens
tttaaataatgtatatctggtactaytagctctcctttttaagtctctttg

SEQ ID NO: 933 - DNA, 51 bp, Homo sapiens
gcattgtgatattaactttcctttayagactccacataatgcatcatgaga

SEQ ID NO: 934 - DNA, 51 bp, Homo sapiens
tagctatttatgaacataaagaccarggtttgaaggcatagtgcatgccca

SEQ ID NO: 935 - DNA, 51 bp, Homo sapiens
gtagagagttaatggagtcagccaamggatgtttgccacagttttccatac

SEQ ID NO: 936 - DNA, 51 bp, Homo sapiens
cttgatatctccccacttccagcatraatctgagtctcagagccacagtgg

SEQ ID NO: 937 - DNA, 51 bp, Homo sapiens
tctgagtgcactaatatgacaattargctataaggaaaaggaagctttaat

SEQ ID NO: 938 - DNA, 51 bp, Homo sapiens
gtagatttgacttgctcttgcctcaytcgacctccaaagtgggactgaaga

SEQ ID NO: 939 - DNA, 51 bp, Homo sapiens
tggttgtaaaaatagtgagaaaatayatatgaaagaagctgccagcatgaa

SEQ ID NO: 940 - DNA, 51 bp, Homo sapiens
cttttgaagaaacacattctactcartaccttcccccatggattggcacaa

SEQ ID NO: 941 - DNA, 51 bp, Homo sapiens
cagaggacttatagggcagtgacackattctgtgtgatgctgtaatggtgg

SEQ ID NO: 942 - DNA, 51 bp, Homo sapiens
cattaaatcactactgtgcatacttwaagccaggtattatctgtgtagtaa

SEQ ID NO: 943 - DNA, 51 bp, Homo sapiens
gaggaaagaaaaaaaatttgatggamagaacttggagattgtgttgctgat

SEQ ID NO: 944 - DNA, 51 bp, Homo sapiens
acaatattctgaagacaatcatttargtgtagaaagaacttccttgaacaa

SEQ ID NO: 945 - DNA, 51 bp, Homo sapiens
tttttcaacttagaaatttatgggartaggacaatttgtttctaatattgc

SEQ ID NO: 946 - DNA, 51 bp, Homo sapiens
aaactaacacatttatttttatgtgyagttaaagtcatagaaacctgtttt

SEQ ID NO: 947 - DNA, 51 bp, Homo sapiens
tttcatctgtgacaggattttaagayggaattggacttttaaaattggtgc

SEQ ID NO: 948 - DNA, 51 bp, Homo sapiens
caaagtctgcgcctctgactagccayatcagtatcattttaataaatttct

SEQ ID NO: 949 - DNA, 51 bp, Homo sapiens
tgcttgaagcaagggttgcataacargtctaggaaacgtctggccgtgtgg

SEQ ID NO: 950 - DNA, 51 bp, Homo sapiens
aattgtatcctctgttgtacataatraaacaagcggtttggcgtgaggcgt

SEQ ID NO: 951 - DNA, 51 bp, Homo sapiens
atcatcccccaatcctcacaaaaaaktaagtctaactataaaacatagacc

SEQ ID NO: 952 - DNA, 51 bp, Homo sapiens
gttcactaaatagtagcagccattaygtatatagtgccacaggagcacaga

SEQ ID NO: 953 - DNA, 51 bp, Homo sapiens
taagaatgattaaagtgaataataaragaatcatagagctatatattaaaa

SEQ ID NO: 954 - DNA, 51 bp, Homo sapiens
acttttaggcagcagagtggtatgaygcgatctgaatttatatcctggggt

SEQ ID NO: 955 - DNA, 51 bp, Homo sapiens
gacattggaatgtatcaattctaacyacatacctgtcttaacaaagcttag

SEQ ID NO: 956 - DNA, 51 bp, Homo sapiens
agactttatgttttttaacactactyacatataacagaggaggaagctttg

SEQ ID NO: 957 - DNA, 51 bp, Homo sapiens
agtaggagacatatttgtattggcawtattccattttgtttgtccccacat

SEQ ID NO: 958 - DNA, 51 bp, Homo sapiens
tagtagggagttgcctagtacatggyatgaaatttgcatttttcctaggcc

SEQ ID NO: 959 - DNA, 51 bp, Homo sapiens
atagacttttagaaaagaaggccacygtatatcaaccattaaaccaaagtc

SEQ ID NO: 960 - DNA, 51 bp, Homo sapiens
tgaggacatggatgatacataactcycataacctataaagtatattgctct

SEQ ID NO: 961 - DNA, 51 bp, Homo sapiens
tttggattttaaaaaatcctactatragtcagacctatgagtcaaagatat

SEQ ID NO: 962 - DNA, 51 bp, Homo sapiens
ttcacatagcactgacaacacttggrattatttttataatttgccttccct

SEQ ID NO: 963 - DNA, 51 bp, Homo sapiens
acccagacctgcagtttactgagaasgtaggtgatccgtggtttggatgga

SEQ ID NO: 964 - DNA, 51 bp, Homo sapiens
ctctggaataatcttcctttactcayatgaactattatcaaccagaaagtg

SEQ ID NO: 965 - DNA, 51 bp, Homo sapiens
acagtgcaaaaggaaacagacctaartccatccagcttaccgttatttatt

SEQ ID NO: 966 - DNA, 51 bp, Homo sapiens
ctgactacaaccacctagaataatcmgagagagtttcaggtgcaaccccta

SEQ ID NO: 967 - DNA, 51 bp, Homo sapiens
cacataagtaggtgattctgtggacyacttagcaaaatctgctaacccagt

SEQ ID NO: 968 - DNA, 51 bp, Homo sapiens
tatttgctaaataatataccatgccracattaacagtgattcttcaaactg

SEQ ID NO: 969 - DNA, 51 bp, Homo sapiens
aaaaaaagtagaagagatttgaacaygtctatataaaaaggaaaaaagaaa

SEQ ID NO: 970 - DNA, 51 bp, Homo sapiens
ttagacaattacttgtgaaaaactayctaaaaatctgtcaagtgaaccatg

SEQ ID NO: 971 - DNA, 51 bp, Homo sapiens
attcattccagccgagcacatgagaragaggtgtggaactggatggtggca

SEQ ID NO: 972 - DNA, 51 bp, Homo sapiens
gatttcaaaacacattgggtattgayagttcaaaaatctaaaaagcaggct

SEQ ID NO: 973 - DNA, 51 bp, Homo sapiens
aaattcctatgaaatacaaatgaaasctaaatctcataatttgaatggatt

SEQ ID NO: 974 - DNA, 51 bp, Homo sapiens
gcattctatacttcttcagcttccayctactgcagttatccagttgtctca

SEQ ID NO: 975 - DNA, 51 bp, Homo sapiens
ttttttatccctcacatatctgaggyaatgaagtgtttaatgagggaactc

SEQ ID NO: 976 - DNA, 51 bp, Homo sapiens
gtgagatttgatggtatgagtcacawctatccctttaaaaaatttttggta

SEQ ID NO: 977 - DNA, 51 bp, Homo sapiens
gaattggtaattcacacggctaaaamtctgacagtctattaaaataccaaa

SEQ ID NO: 978 - DNA, 51 bp, Homo sapiens
ttgtacaatctccaaaagtcctataratgaagttgttgcacattttttagg

SEQ ID NO: 979 - DNA, 51 bp, Homo sapiens
gcagcagataagaatcttaggcaagyaaagggctagcactagcgaactggg

SEQ ID NO: 980 - DNA, 51 bp, Homo sapiens
tccacatttaaactctatgcgttcawgatcccacgaaagctgttctttctc

SEQ ID NO: 981 - DNA, 51 bp, Homo sapiens
tcccagccatttagtttgctaaatawcattgggtagacgttttttatacat

SEQ ID NO: 982 - DNA, 51 bp, Homo sapiens
gtgctgttaacctaaacagctctaayaatcaagcacattgcctctgataca

SEQ ID NO: 983 - DNA, 51 bp, Homo sapiens
aattttattttgcctgaaataattarattgtttgtttctgagaagctgtat

SEQ ID NO: 984 - DNA, 51 bp, Homo sapiens
cagctcaaggctaaaagtgaacacastccgtataactgaactaatggtttc

SEQ ID NO: 985 - DNA, 51 bp, Homo sapiens
aatatttccataaagactcgatgaartaaaacaaaacaaaaatttttgatt

SEQ ID NO: 986 - DNA, 51 bp, Homo sapiens
aagaattcattccgagtttggtgtckaattctctcaaatcaaccagccaga

SEQ ID NO: 987 - DNA, 51 bp, Homo sapiens
acacaggatttcagtttggggcttawgtaagaaactagcaagttatgtgaa

SEQ ID NO: 988 - DNA, 51 bp, Homo sapiens
cgaacttatcatgacctttggttaayttaacatactcctgtagtctttcat

SEQ ID NO: 989 - DNA, 51 bp, Homo sapiens
agtcattgagttccttccacagagcsctggagataaaacagctgcaatgct

SEQ ID NO: 990 - DNA, 51 bp, Homo sapiens
ttgaaatttcactagtcttatctatyagagttttagcacaaaacagatcca

SEQ ID NO: 991 - DNA, 51 bp, Homo sapiens
atcccctgtgacttcctgcataggaygggtacttagtaaatgctcaataaa

SEQ ID NO: 992 - DNA, 51 bp, Homo sapiens
agccaaagtttatttttgttgttggkattattttgttacattcagacagag

SEQ ID NO: 993 - DNA, 51 bp, Homo sapiens
ttatgtatactcacaaacggcaatastatgcagcaatgaaatgaataaact

SEQ ID NO: 994 - DNA, 51 bp, Homo sapiens
tttctaatatttgaagatacatcacrcctgcagaattatgcactccattct

SEQ ID NO: 995 - DNA, 51 bp, Homo sapiens
ttatagtgacttgcttatgtgtcttygtctttaacatcaaaggcaaggacc

SEQ ID NO: 996 - DNA, 51 bp, Homo sapiens
cagtgcaactcaaccattttattgargcccatctaagaatcttgcacaggg

SEQ ID NO: 997 - DNA, 51 bp, Homo sapiens
ccctctactcagagctggtttatgargtcccagagtggctgagctctgtca

SEQ ID NO: 998 - DNA, 51 bp, Homo sapiens
acgatctctaatgtatcatggtttayaaatgagtctgaagtgacgcttggt

SEQ ID NO: 999 - DNA, 51 bp, Homo sapiens
catctgcagaggctaaatgcagaccyattgtttatttttgttgtgaccttg

SEQ ID NO: 1000 - DNA, 51 bp, Homo sapiens
atgactgtgtttccctggaaaattaygtaacttctctaggcttcagtttct

SEQ ID NO: 1001 - DNA, 51 bp, Homo sapiens
gctcatagtttagttccacaagctaytaagcaatgagaatcttgagaaacc

SEQ ID NO: 1002 - DNA, 51 bp, Homo sapiens
catgaggaggaggaaaaatgctagcraaagattcattggaggttaaataac

SEQ ID NO: 1003 - DNA, 51 bp, Homo sapiens
tgatatgacacagacattgggattaycagtgaatttaaagtaattacaaat

SEQ ID NO: 1004 - DNA, 51 bp, Homo sapiens
agatagcaagatatgttatttaaacygtgtgttatagatggaaaataaata

SEQ ID NO: 1005 - DNA, 51 bp, Homo sapiens
aaactaacaaagggctaaagtttacrtcataaagtgtcaatgcataaatct

SEQ ID NO: 1006 - DNA, 51 bp, Homo sapiens
acctaatttcacatatatgaagtgasagcaagtaaggcaatcttagagaaa

SEQ ID NO: 1007 - DNA, 33 bp, Homo sapiens
aagaccatcctcatgcycaatgatttaccagaa

SEQ ID NO: 1008 - DNA, 51 bp, Homo sapiens
ttttaactaaatatgcgtagcttaartacaaaataagtttcaaaaatgaat

SEQ ID NO: 1009 - DNA, 51 bp, Homo sapiens
tcacagcaatgctataaaataggtaygcttgttatcttcattttactgaga

SEQ ID NO: 1010 - DNA, 51 bp, Homo sapiens
aaaacaatcttacatagcagctcagkctataaaatatataagcagaaaaca

SEQ ID NO: 1011 - DNA, 51 bp, Homo sapiens
aatcaataaatctcagctgttattayatcattagtatcaggcatccagctg

SEQ ID NO: 1012 - DNA, 51 bp, Homo sapiens
acctcgaaagcattatattgagttawagaagttagataagaaagccacata

SEQ ID NO: 1013 - DNA, 51 bp, Homo sapiens
ggtgaaactgtagccaaaactcttayaaattctatggtggacatttggtga

SEQ ID NO: 1014 - DNA, 51 bp, Homo sapiens
aaatagaaatgggacagttactagcrgcaaattttggggctatgttagggt

SEQ ID NO: 1015 - DNA, 51 bp, Homo sapiens
caaccagtctgtttgtcattgttcayggttttgtacttcattatctgaata

SEQ ID NO: 1016 - DNA, 51 bp, Homo sapiens
tagataaaacatgtagagaagaatcrcatttttggagacacaaaccagttt

SEQ ID NO: 1017 - DNA, 51 bp, Homo sapiens
ggagtagttactgcaccatcatggasactcttacaggagatgaaaggcgca

SEQ ID NO: 1018 - DNA, 51 bp, Homo sapiens
gtgagccaccatgcccggctactaartgcagtattccaacatttgggacac

SEQ ID NO: 1019 - DNA, 51 bp, Homo sapiens
ccttctggtgccctcagtacttatcwccgtctgatatcttgtgtcttttct

SEQ ID NO: 1020 - DNA, 51 bp, Homo sapiens
aaaagataacctagttctgtacaaayttagctcactaaaccagaaagaaca

SEQ ID NO: 1021 - DNA, 51 bp, Homo sapiens
cagccttttacacatctgtcaattgragaagttaacctctctgctgttttg

SEQ ID NO: 1022 - DNA, 51 bp, Homo sapiens
taatatagatggaaaaaaagaaggcsaaatatgtgccagacttactcattt

SEQ ID NO: 1023 - DNA, 51 bp, Homo sapiens
ggaccacacactccagtactgagccratggttgatagggaattgcatctga

SEQ ID NO: 1024 - DNA, 51 bp, Homo sapiens
gaggtctttcactagtactgatgaastccctgaaattacaagcaaaaatgt

SEQ ID NO: 1025 - DNA, 51 bp, Homo sapiens
aataaaaatgaaaataaatgtataarataacttctcattctagtacagtta

SEQ ID NO: 1026 - DNA, 51 bp, Homo sapiens
aattaaaaaaactcttgcaatactcyagaaatttttttatatttgttttaa

SEQ ID NO: 1027 - DNA, 51 bp, Homo sapiens
ttaatctgttccctggaattccacaraccaaatctaatctcctcttcactt

SEQ ID NO: 1028 - DNA, 51 bp, Homo sapiens
attggatagcttcagaaggcctgcaygttttaaaatttttggtatttttca

SEQ ID NO: 1029 - DNA, 51 bp, Homo sapiens
ttgcttccacatgtcagctgaaaccyaatattagctatatctgctggaatc

SEQ ID NO: 1030 - DNA, 51 bp, Homo sapiens
gtattggtgttattaaaaatggctasagaaagtggatggtagaatagatga

SEQ ID NO: 1031 - DNA, 51 bp, Homo sapiens
ttcatatagatatactgcatatcaayaaattacatataacgtgtctagaat

SEQ ID NO: 1032 - DNA, 51 bp, Homo sapiens
tgctgtttgatgggtgacacctgtawcttatgtctgtgcctatttcactga

SEQ ID NO: 1033 - DNA, 51 bp, Homo sapiens
caaggagcaactggaaagtttgccaygactaaggtgcacaatggggtcaga

SEQ ID NO: 1034 - DNA, 51 bp, Homo sapiens
gatagaacttttgaatccaaaaaggraatacacaaaattgaaatatggaga

SEQ ID NO: 1035 - DNA, 51 bp, Homo sapiens
acatctcctttctttggcttggacckacctcatgggtgtccctttccacag

SEQ ID NO: 1036 - DNA, 51 bp, Homo sapiens
agtaatttattttgagattccatcartaatcagatcttgctaaatttacat

SEQ ID NO: 1037 - DNA, 51 bp, Homo sapiens
gctcttccaataagggaatcatgcamgttttttagatcagcagaaaaacac

SEQ ID NO: 1038 - DNA, 51 bp, Homo sapiens
atggatctaggttgaatgatgtcaaygggaatttgtttctcttcatatttc

SEQ ID NO: 1039 - DNA, 51 bp, Homo sapiens
gatgaattagaacataagagttatayaatttgttgtcttttttgtatatta

SEQ ID NO: 1040 - DNA, 51 bp, Homo sapiens
aataaacaatccttagtttagatagkaatttggaaaggagaggtgaaaggg

SEQ ID NO: 1041 - DNA, 51 bp, Homo sapiens
ggccagagttaaacattgaggaatgracctaaaatgttgactaaaagatga

SEQ ID NO: 1042 - DNA, 51 bp, Homo sapiens
acaatgtcctgaaaggcagctaagaktactttagactcacgactagtgtct

SEQ ID NO: 1043 - DNA, 51 bp, Homo sapiens
tcttctatcactttataattacacamgctaataggattcattccatcacac

SEQ ID NO: 1044 - DNA, 51 bp, Homo sapiens
cagcatttatgtgctttagtatcctyacctcttgacatgaggatgtgaaat

SEQ ID NO: 1045 - DNA, 51 bp, Homo sapiens
agaaatttagacctgaaatgcaaaaktgaggtcaacagaactataattact

SEQ ID NO: 1046 - DNA, 51 bp, Homo sapiens
gcaaagattaactcatctcagtaaakagcgttaagaactgcggtaggctga

SEQ ID NO: 1047 - DNA, 51 bp, Homo sapiens
aagcactactttattatgtgtttgcrgagaactactagatggtctggggat

SEQ ID NO: 1048 - DNA, 51 bp, Homo sapiens
atgcctgttcactgacagctctaaaygtggtattctgtgatagggacagga

SEQ ID NO: 1049 - DNA, 51 bp, Homo sapiens
cctgcttaatctggaagctaattgamagctagaaatttggaaagacatagt

SEQ ID NO: 1050 - DNA, 51 bp, Homo sapiens
gtaagtaccatattacttttcacaaygtacggatgaagaaacaagaatgcc

SEQ ID NO: 1051 - DNA, 51 bp, Homo sapiens
tggaaaacaagaaaaattggagaacrataactgacatgtaaaaacattgtt

SEQ ID NO: 1052 - DNA, 51 bp, Homo sapiens
ccataaagttagttaataaagcagamgcaaatttagaaaggattgactcta

SEQ ID NO: 1053 - DNA, 51 bp, Homo sapiens
taagctgtgggagttgagggtttaaytgcttggccacttggccttctctct

SEQ ID NO: 1054 - DNA, 51 bp, Homo sapiens
gctttaccagatgccgaatacccatkactgtttaggtctatttctagactt

SEQ ID NO: 1055 - DNA, 51 bp, Homo sapiens
gaattattgaagagtttgaagaacarctacttagaaatgataataaacact

SEQ ID NO: 1056 - DNA, 51 bp, Homo sapiens
gaaggagaaaattaaaccattgcacygggagctgaaaatatacagagaaag

SEQ ID NO: 1057 - DNA, 51 bp, Homo sapiens
gcatgagctaccatgcctaactccayaaagattaatttttggtgatgagaa

SEQ ID NO: 1058 - DNA, 51 bp, Homo sapiens
agtgccacacacaggcgagtgttaaygttttaaagcgaaaaagttctgcat

SEQ ID NO: 1059 - DNA, 51 bp, Homo sapiens
aggcgagccaagcatggcaaagaacsagctgggcacgaagatgccagaccc

SEQ ID NO: 1060 - DNA, 51 bp, Homo sapiens
ctctgagaaaactagaatctcttccrgctataaagaatgtacttaaatata

SEQ ID NO: 1061 - DNA, 51 bp, Homo sapiens
ccttataaaggcctgttcagcaataytaacttttcagttactttgaaatgt

SEQ ID NO: 1062 - DNA, 51 bp, Homo sapiens
tattctagttgtttctttatgcaggyatttagtagaatctcacttgtcctt

SEQ ID NO: 1063 - DNA, 51 bp, Homo sapiens
ataacctgcaaatcatgcagaaaaartataatcatccctcagtatccaggg

SEQ ID NO: 1064 - DNA, 51 bp, Homo sapiens
tgctaatgagttcctgttgataaaakgcttagatcagtgctcaggatgtag

SEQ ID NO: 1065 - DNA, 51 bp, Homo sapiens
gctcccagtgacatctctcttcaccratttgcccaagtcagtttgctgagt

SEQ ID NO: 1066 - DNA, 51 bp, Homo sapiens
agccctttttgcctgcaaacaaataygtactcctcaggggaacattccatt

SEQ ID NO: 1067 - DNA, 51 bp, Homo sapiens
gatttctaatttaagaattgtatcawcttgaagtagtgtgaaatgtgagaa

SEQ ID NO: 1068 - DNA, 51 bp, Homo sapiens
tgtgtttcttgcagaataaatctgartactcggtcaatattttaggtagta

SEQ ID NO: 1069 - DNA, 51 bp, Homo sapiens
ttccatagaaacagattttatgacayggtaatcagttgctggtgtcttttt

SEQ ID NO: 1070 - DNA, 51 bp, Homo sapiens
tttgtggcataattctatagtaaagraaattgatgtaatttagtccaaatt

SEQ ID NO: 1071 - DNA, 51 bp, Homo sapiens
tactttctattcaggaggaactatgragagtacaataatgaagaagttata

SEQ ID NO: 1072 - DNA, 51 bp, Homo sapiens
ttgcatagaaaagcatgtggaatcaygaacgcacaatgtccggctcttacg

SEQ ID NO: 1073 - DNA, 51 bp, Homo sapiens
cattttgatcatgataacctggacaycgattggtttttctgaagcaacttg

SEQ ID NO: 1074 - DNA, 51 bp, Homo sapiens
taggtcagctaagatgcctgcaatayagttcaggagatttaagaaggcata

SEQ ID NO: 1075 - DNA, 51 bp, Homo sapiens
tttttatagtacactcatcttagaargtattattcaataaatgaagaaaaa

SEQ ID NO: 1076 - DNA, 51 bp, Homo sapiens
ataggaagttctctgtgaccttttcycaggtaagaatcaagtgattccatt

SEQ ID NO: 1077 - DNA, 51 bp, Homo sapiens
ttctttcctcagagctgcgtcaagaratacaataataagtaatgccattag

SEQ ID NO: 1078 - DNA, 51 bp, Homo sapiens
aaaggtgcttataaagcattgactaygccaggtacttcacttcctatcccc

SEQ ID NO: 1079 - DNA, 51 bp, Homo sapiens
acgagccagtttgtagtacctgccaygtaggattgccagataaaaccaaag

SEQ ID NO: 1080 - DNA, 51 bp, Homo sapiens
atctttctagattatctctcttcacyctaacttcatctgtttttctctgta

SEQ ID NO: 1081 - DNA, 51 bp, Homo sapiens
attttgtgtagtgttgggtttataargttgaatactggctaggagctatta

SEQ ID NO: 1082 - DNA, 51 bp, Homo sapiens
tttgttatcaacacgttattaagaawgggcaagatgtccttatatactaga

SEQ ID NO: 1083 - DNA, 51 bp, Homo sapiens
actctcaaaaaacatattatgcaaarccacttgagatttaccagaatgttt

SEQ ID NO: 1084 - DNA, 51 bp, Homo sapiens
ttgatcctgtggggccttgacaaagyattgattgttccttcaaccttcaga

SEQ ID NO: 1085 - DNA, 51 bp, Homo sapiens
tttgtttttgtttttccagtcgctgyatggaccatcatatgtaaaagtggg

SEQ ID NO: 1086 - DNA, 51 bp, Homo sapiens
tatataagttggatattgggcttcamtcacaagtagaccatggcttaagat

SEQ ID NO: 1087 - DNA, 51 bp, Homo sapiens
taaccagacatcttattttagaataygaagaatccagtaatctcttcaagt

SEQ ID NO: 1088 - DNA, 51 bp, Homo sapiens
tctggttacaatagtggctgtaacayctcctaaagttattgtgagactttg

SEQ ID NO: 1089 - DNA, 51 bp, Homo sapiens
tgtcaaaacttgacaaacaatttaartgaagaactgaagtatagctgcaat

SEQ ID NO: 1090 - DNA, 51 bp, Homo sapiens
ctttcccacttggatacaatattcayctttgtgttaggaggtagagatatt

SEQ ID NO: 1091 - DNA, 51 bp, Homo sapiens
gctgaccaatgggagctagagtcaaycttagtgatgcttacccaccacagt

SEQ ID NO: 1092 - DNA, 51 bp, Homo sapiens
taaaaaagctgacagatggaactagsaaatgaattgtaaaatcaagtaagg

SEQ ID NO: 1093 - DNA, 51 bp, Homo sapiens
cctaaacatagttttcatttgatgaygtatttaagaggcaaagtataaggc

SEQ ID NO: 1094 - DNA, 51 bp, Homo sapiens
tgcattgcattctgctctacactacrggcctaattggcacagagcctggca

SEQ ID NO: 1095 - DNA, 51 bp, Homo sapiens
aataatttcctttgcacgtctaaackgagatgctcagaagtttgctaatac

SEQ ID NO: 1096 - DNA, 51 bp, Homo sapiens
ctattggaccgtaccttgtgattccrcgagtcaacactccttaataaactc

SEQ ID NO: 1097 - DNA, 51 bp, Homo sapiens
gggagggatgcatgggctgaactgakccaataagcttatttctcttaagaa

SEQ ID NO: 1098 - DNA, 51 bp, Homo sapiens
tttatattttcttttccactaaagaygttaagtcagaaaataatatctaga

SEQ ID NO: 1099 - DNA, 51 bp, Homo sapiens
ccaggttgagtgtgtttctgtaaaaytgcactgataggatcctgaatcacc

SEQ ID NO: 1100 - DNA, 51 bp, Homo sapiens
aatttcattgtgaagagatggatcaygtgaggaaagtaattccatctcaga

SEQ ID NO: 1101 - DNA, 51 bp, Homo sapiens
cattgccagaatcttctgtaggacartcttactagaaatcagtaatcctga

SEQ ID NO: 1102 - DNA, 51 bp, Homo sapiens
gaccacattttgagaaccactggtayggaggaagtaaactacttgagactg

SEQ ID NO: 1103 - DNA, 51 bp, Homo sapiens
atgccttagcaatagatttgcaccayatatcttgaggatacgatattttat

SEQ ID NO: 1104 - DNA, 51 bp, Homo sapiens
aggggttaaatatcagtcataagatyaataatggtagagttaggacaattt

SEQ ID NO: 1105 - DNA, 51 bp, Homo sapiens
tgtgctttcttatgaaataaatctaracaggaagcctggttttgctacgta

SEQ ID NO: 1106 - DNA, 51 bp, Homo sapiens
gtcttcaaatgactacgagaaaccamgcacatggatttgggggatacacat

SEQ ID NO: 1107 - DNA, 51 bp, Homo sapiens
ttcacatcagaaacattttgccactrtcttcttaggatacatatgtccatt

SEQ ID NO: 1108 - DNA, 51 bp, Homo sapiens
tatctcgtaaagtttttcacataaakaatatatcagttaaagaacatacta

SEQ ID NO: 1109 - DNA, 51 bp, Homo sapiens
tacatttccttccatcttagggaagragttaaataaatctcaagctccaag

SEQ ID NO: 1110 - DNA, 51 bp, Homo sapiens
aaagaaattggtcactgaccagaaaycttgagcttgacttacaggatggta

SEQ ID NO: 1111 - DNA, 51 bp, Homo sapiens
aggagggaactgccgttgttcctacrctgcagatgaagaaacaaaaggctc

SEQ ID NO: 1112 - DNA, 51 bp, Homo sapiens
tgaggccatgtgaattacgataagaygcatggtaaattctcagagaaagga

SEQ ID NO: 1113 - DNA, 51 bp, Homo sapiens
taggtccaatttagatagcaaagaamcatttattgatgtgatgtattgctg

SEQ ID NO: 1114 - DNA, 51 bp, Homo sapiens
gaaatgtgaagaccttcagtgtatcscatttaaattgacttgcaaggctta

SEQ ID NO: 1115 - DNA, 51 bp, Homo sapiens
agaaaccagaaatgatctagtaagaytaaattgtgcctaaagtaatggaaa

SEQ ID NO: 1116 - DNA, 51 bp, Homo sapiens
ttcagtgtaagcacatagtgtaacawcttgtctaatgtattttgggctatc

SEQ ID NO: 1117 - DNA, 51 bp, Homo sapiens
tgcatttaagatgacctggagaatakacagcatactccgtagttattgagt

SEQ ID NO: 1118 - DNA, 51 bp, Homo sapiens
gtctgctgttaaatactctggctgtractcactgaaactaacaggcacagg

SEQ ID NO: 1119 - DNA, 33 bp, Homo sapiens
aagaagcttcatacccrttagcggtcactttcc

SEQ ID NO: 1120 - DNA, 51 bp, Homo sapiens
gaagccaccatcccagaaatgccaayggctacagacaaaaatccccaatta

SEQ ID NO: 1121 - DNA, 51 bp, Homo sapiens
ttcacctatttttatgcctgggatartatagtattttataaccagaccaca

SEQ ID NO: 1122 - DNA, 51 bp, Homo sapiens
tatgaaaaggctgtgaagctgaagaraaactaagaaatggatattgctgca

SEQ ID NO: 1123 - DNA, 51 bp, Homo sapiens
gtttaaggttaaactactaaaattaygaaatatctcttttgaacaaatcac

SEQ ID NO: 1124 - DNA, 51 bp, Homo sapiens
ttcaggccacgcatgtaactcagggycagttctcttaatgctctcatttta

SEQ ID NO: 1125 - DNA, 51 bp, Homo sapiens
tcagatatctcatttccaagaataargtaagtacccctttgctctcatggt

SEQ ID NO: 1126 - DNA, 51 bp, Homo sapiens
aagattccaaaacagccaggtatcayagagtgcttaagttctaagtggagg

SEQ ID NO: 1127 - DNA, 51 bp, Homo sapiens
gagataattcaacactatgtcctcartatccaattcacggcctggctcaca

SEQ ID NO: 1128 - DNA, 51 bp, Homo sapiens
tatctaagtgcactatgcctgtgatktgactaacttttataatcttccata

SEQ ID NO: 1129 - DNA, 51 bp, Homo sapiens
tacagctagatcattttggggtcagrctccattgattttttctttgtatta

SEQ ID NO: 1130 - DNA, 51 bp, Homo sapiens
ggcagagagaaggatttgggagatayttcagagggagaagcaagaggacgt

SEQ ID NO: 1131 - DNA, 51 bp, Homo sapiens
tcacagcagttcaggcgtaaaattayctgcaatttgtgagcaggtcttaga

SEQ ID NO: 1132 - DNA, 51 bp, Homo sapiens
aacatttataacaaacttaacatacrgtaagcaatcaatcaatgataactg

SEQ ID NO: 1133 - DNA, 51 bp, Homo sapiens
attataatcggggttttctattaaartctccactaattgcatgattctcac

SEQ ID NO: 1134 - DNA, 51 bp, Homo sapiens
gcagaaattacataggtaaaggggaygcgtttgacaaatgggtgcgcccat

SEQ ID NO: 1135 - DNA, 51 bp, Homo sapiens
gtcaatggacctctcataatggcacmgacaggagatatttgtgttttgtgt

SEQ ID NO: 1136 - DNA, 51 bp, Homo sapiens
taaggaaataaatggtaattaaagtsatgagagtggacaatttagccctcg

SEQ ID NO: 1137 - DNA, 51 bp, Homo sapiens
aaggtggtcaattacaaggttggaayaatggtccaggtgagaaggatgaag

SEQ ID NO: 1138 - DNA, 51 bp, Homo sapiens
ttcacagtgaatgacagaatgccaartcttctaggcctggaataaataaat

SEQ ID NO: 1139 - DNA, 51 bp, Homo sapiens
ggctagtgcatgctgtaggcatgatsttgtggtctgaaccagcagcctcct

SEQ ID NO: 1140 - DNA, 51 bp, Homo sapiens
attttgtgttccattgctcctttgakaaataatcactcaccacctcagccc

SEQ ID NO: 1141 - DNA, 51 bp, Homo sapiens
atggttttcctctccagcacccattkcctccttactcaagcaatggccctg

SEQ ID NO: 1142 - DNA, 51 bp, Homo sapiens
gcaggattaaaaacaatgactcatayagtgagaaagtgattgctattccgg

SEQ ID NO: 1143 - DNA, 33 bp, Homo sapiens
aaatagagtatatgacyagcacagttttcacat

SEQ ID NO: 1144 - DNA, 51 bp, Homo sapiens
aattagtatttacctgtatcttttgyagtgtttaaattttctaaaatgtgt

SEQ ID NO: 1145 - DNA, 51 bp, Homo sapiens
aggtaagtattagggagtgaggatgsaaaaatagaaaatgtccagaacagt

SEQ ID NO: 1146 - DNA, 51 bp, Homo sapiens
gttataactatcagatctcagtgatyaatttggtttatgatgcagaaaacc

SEQ ID NO: 1147 - DNA, 51 bp, Homo sapiens
cttgcaatgcaggggcacactaaacrtaagcaatagtaattcatccatttt

SEQ ID NO: 1148 - DNA, 51 bp, Homo sapiens
aggtctggggattaataattgcatamgcatctatataatttacatatattt

SEQ ID NO: 1149 - DNA, 51 bp, Homo sapiens
atagatttttttaaatgtaatttaayttaaagttctagaatacaagtgcag

SEQ ID NO: 1150 - DNA, 51 bp, Homo sapiens
taagacttctcaccacctaattaatragaaataactgaacactaatatgag

SEQ ID NO: 1151 - DNA, 51 bp, Homo sapiens
atacattttttggcttgagctacaayaaaaataaagttaggaaaagctgtc

SEQ ID NO: 1152 - DNA, 51 bp, Homo sapiens
caaatccagagaagtaacgtgcccartccttaacaatgaaatgtgtaacca

SEQ ID NO: 1153 - DNA, 51 bp, Homo sapiens
ttatgatatataaaatccataaaagragagagtcatgcttaaaattatgca

SEQ ID NO: 1154 - DNA, 51 bp, Homo sapiens
ctgcccttcatctccacaggctcaaktaataaaagttatgcataactcatg

SEQ ID NO: 1155 - DNA, 51 bp, Homo sapiens
tgggccagagttgctgagaagctaaraaaactcaagctgcagggacctgca

SEQ ID NO: 1156 - DNA, 51 bp, Homo sapiens
atccttttaacaagacaagccactcractaatacatgctctgaaggagcga

SEQ ID NO: 1157 - DNA, 51 bp, Homo sapiens
gatgaaagcagagaaacttgataccracctagctaaggataaatgtatggt

SEQ ID NO: 1158 - DNA, 51 bp, Homo sapiens
cttttagaaaatcctttagttccaaycatgaagcattccaatgtctcagaa

SEQ ID NO: 1159 - DNA, 51 bp, Homo sapiens
ccctggtcctctgtatatactactaygattttccctatcttatcttgtact

SEQ ID NO: 1160 - DNA, 51 bp, Homo sapiens
ccccagagtacttgactgttgatgtyaaaaagcataggcatagctactcgg

SEQ ID NO: 1161 - DNA, 51 bp, Homo sapiens
caaacatgtattttgtggtttttaartccagtcaccattaataattcctac

SEQ ID NO: 1162 - DNA, 51 bp, Homo sapiens
aactgcaattacaaccatattcaaamaagttttgtctaattcctcttctta

SEQ ID NO: 1163 - DNA, 51 bp, Homo sapiens
taagctatgtgacttttgattttgarattttgaatcatgtgtcttttgaat

SEQ ID NO: 1164 - DNA, 51 bp, Homo sapiens
ttattgttatgttagcacattaaaartaccccaattaggtaaattattatg

SEQ ID NO: 1165 - DNA, 51 bp, Homo sapiens
aaatgctgacatctgaccgcttgttyactttatgtgtcaactcctgggctt

SEQ ID NO: 1166 - DNA, 51 bp, Homo sapiens
tgtctctctcaatcctgttaagtcayagcttagttttgtcactgcatcctt

SEQ ID NO: 1167 - DNA, 51 bp, Homo sapiens
aaagtgtacacatccctgttcaaccsagatgtttattgtcatgggtgtggc

SEQ ID NO: 1168 - DNA, 51 bp, Homo sapiens
ggatatgggtctgttctgtgcaggaygtaaaggatgataatagagaacgga

SEQ ID NO: 1169 - DNA, 51 bp, Homo sapiens
catagatatatctcatttaatcctamagcagtcctacaataaaaatgttat

SEQ ID NO: 1170 - DNA, 51 bp, Homo sapiens
tccacgacaaagacagctcaacccaytggaacaaacagactcccaatgtgg

SEQ ID NO: 1171 - DNA, 51 bp, Homo sapiens
tatgtataatttgggaatagtataasctctcaatttcattgttaggattag

SEQ ID NO: 1172 - DNA, 51 bp, Homo sapiens
gttaaacaagaatcacaacgggagtraataatcatatcatgattaagaata

SEQ ID NO: 1173 - DNA, 51 bp, Homo sapiens
atcagggcacaagtgactttcatcarggttgaaactggcagaagaaaaagc

SEQ ID NO: 1174 - DNA, 51 bp, Homo sapiens
ctagaacctggatcagggtttcagargacattgttgttatggagatagagg

SEQ ID NO: 1175 - DNA, 51 bp, Homo sapiens
ctagttagaagttttctaattggtasagtggtgatggtagtgtcttttaca

SEQ ID NO: 1176 - DNA, 51 bp, Homo sapiens
tcaactgctttaaaattccacttccraagataccatatgaatctaaataaa

SEQ ID NO: 1177 - DNA, 51 bp, Homo sapiens
ctcatacatacacatttatctaaaawttaattggttctcttaatagactca

SEQ ID NO: 1178 - DNA, 51 bp, Homo sapiens
taatgtgtaatgaaactaaagtatasgagaatcaaactcaaaacccttttc

SEQ ID NO: 1179 - DNA, 51 bp, Homo sapiens
aaaagatgaagcttacatgaccttcyaaaattgaatatgacagtatcctcc

SEQ ID NO: 1180 - DNA, 51 bp, Homo sapiens
tttaagaacttgaaatagcatgaccracactatcttcattagaacagaatg

SEQ ID NO: 1181 - DNA, 51 bp, Homo sapiens
cattgtggtagtaggagatgaaagaygaatcagacctagaacctgcccctg

SEQ ID NO: 1182 - DNA, 51 bp, Homo sapiens
tcagatccactttaattctagctatraacacagaacatgcaaaactgtttt

SEQ ID NO: 1183 - DNA, 51 bp, Homo sapiens
atttttaaaaaattctatttcccacygtattttgttttaagaaaatgtcag

SEQ ID NO: 1184 - DNA, 51 bp, Homo sapiens
atcagttcccacaatctttaaataamgtccaggataaagtccaaataaagt

SEQ ID NO: 1185 - DNA, 51 bp, Homo sapiens
cagctataagactgtaacccacatayacctgtctgcttcctattttgggag

SEQ ID NO: 1186 - DNA, 51 bp, Homo sapiens
aattcataattttcaaatggaaatartaactctcaatgcagacaggtttga

SEQ ID NO: 1187 - DNA, 51 bp, Homo sapiens
ggaaagtttatgatttctatccttcrcgtctccaaaagaaaccaagctcct

SEQ ID NO: 1188 - DNA, 51 bp, Homo sapiens
ctttgaaaattaaggattgtgagaamtagagaattgttaggagtgatactt

SEQ ID NO: 1189 - DNA, 51 bp, Homo sapiens
tagcggtcattagcaaagtaactaartaaaatacagtgctggtagtcaaaa

SEQ ID NO: 1190 - DNA, 51 bp, Homo sapiens
gatgagtcttccccattgtcaatatragaattacagaaaacttaaatttta

SEQ ID NO: 1191 - DNA, 51 bp, Homo sapiens
caatcagagttattgtgatagttaartgagagattatgaggatcttattca

SEQ ID NO: 1192 - DNA, 51 bp, Homo sapiens
ccattttctgtatggaaactccaacraagctatgtcaaggagatggcccga

SEQ ID NO: 1193 - DNA, 51 bp, Homo sapiens
agaaacaaaaaaaagacaagttggasataagagttagaaaaaaaacagagt

SEQ ID NO: 1194 - DNA, 51 bp, Homo sapiens
atcattttgttgcaagcaagagaaaytccctaggctagaggaaagaaacag

SEQ ID NO: 1195 - DNA, 51 bp, Homo sapiens
gtatcatatttaggatcatatatcaytccttctgaagggggaaaaaaacca

SEQ ID NO: 1196 - DNA, 51 bp, Homo sapiens
cccacccacagcagtgtctcaaatgyaccctttctggtcatgccagggaat

SEQ ID NO: 1197 - DNA, 51 bp, Homo sapiens
tggggatccaatcaaaatgtaaacaraggcactgtagagaagacagagact

SEQ ID NO: 1198 - DNA, 51 bp, Homo sapiens
attcggaaggaatatcaaaacctgcrccatgggttgaatgcaaagcagttt

SEQ ID NO: 1199 - DNA, 51 bp, Homo sapiens
taagaaaaatatgagaggaaatcaasgacgagtctcaggtttctagtttag

SEQ ID NO: 1200 - DNA, 51 bp, Homo sapiens
agactccgctcattctagtgccttcmaccaaggatatcttcctgagttttg

SEQ ID NO: 1201 - DNA, 51 bp, Homo sapiens
ggggttgagttaaggcattgactcakggacatgttgatctgatcagttaac

SEQ ID NO: 1202 - DNA, 51 bp, Homo sapiens
ttatcatgtgggagggcatctaatcyacctatcctcttgctgatgatcttg

SEQ ID NO: 1203 - DNA, 51 bp, Homo sapiens
gagaaacgaaaacaagctattttccrcgcccccctgtgaaaggcaggtccc

SEQ ID NO: 1204 - DNA, 51 bp, Homo sapiens
ccctcaagatcctacacattccagakaagagagcctacttcagagcttagt

SEQ ID NO: 1205 - DNA, 51 bp, Homo sapiens
aatttctagaatctgatattccacaratgactaatgtggttcaaatatctg

SEQ ID NO: 1206 - DNA, 51 bp, Homo sapiens
cctaaaattgttggagagtgctattmaaaatgattataaaaatatgtgtgg

SEQ ID NO: 1207 - DNA, 51 bp, Homo sapiens
ccttgttacatgagtaataacaaacsctagcggaatggatgagatcactta

SEQ ID NO: 1208 - DNA, 51 bp, Homo sapiens
taggcactcaataaatatgtattgayagactgattagacaatgctttgtta

SEQ ID NO: 1209 - DNA, 51 bp, Homo sapiens
ttctcacttttgtaatcactggctaygttgacaataacccatgctctctct

SEQ ID NO: 1210 - DNA, 51 bp, Homo sapiens
aggtggaagcggtcctgaacagagakacagtcgtctgtgagctctgacatt

SEQ ID NO: 1211 - DNA, 51 bp, Homo sapiens
gatgcaagccgtattaaacaagtaasggaactctacaggatgcagaattac

SEQ ID NO: 1212 - DNA, 51 bp, Homo sapiens
acttcaccgatttggtgtgtgtcaayttccatctgctagtatctgcatctc

SEQ ID NO: 1213 - DNA, 51 bp, Homo sapiens
taaatttgcacaaaaaggactataamatgaggcaaaatggtaaattctata

SEQ ID NO: 1214 - DNA, 51 bp, Homo sapiens
attaataaaagacttcattttaaaastccttttctgatactgaaagcctgc

SEQ ID NO: 1215 - DNA, 51 bp, Homo sapiens
caaaaaagatataaatgccacttacyaagtctttatgaatagtgcttgagt

SEQ ID NO: 1216 - DNA, 51 bp, Homo sapiens
tcttgagttttgatatgaattatgaraactgagaatttgacactgacctaa

SEQ ID NO: 1217 - DNA, 51 bp, Homo sapiens
gtttcaggaagcttgtccaggtatcyatgagacaatcatcaaatcgttgag

SEQ ID NO: 1218 - DNA, 51 bp, Homo sapiens
cttcccctaaatggaattccacttaygatgcattttgtcagccagatttca

SEQ ID NO: 1219 - DNA, 51 bp, Homo sapiens
aagtttccttctaaatgaattgatasggtgtaatcttttaaagaatcatga

SEQ ID NO: 1220 - DNA, 51 bp, Homo sapiens
atatctaaatctcttgctcctctaartcaagtaatctgtcaagctcttaca

SEQ ID NO: 1221 - DNA, 51 bp, Homo sapiens
tcccttcagagactgcgttccttgayggtatggcccatgatctgctgcagt

SEQ ID NO: 1222 - DNA, 51 bp, Homo sapiens
agaactgggctgacattaacacagartctatccttgaagaaaaaaacaact

SEQ ID NO: 1223 - DNA, 51 bp, Homo sapiens
ttggtacaaagcagtagtatttgcaygtatcaactagttaatagagtggat

SEQ ID NO: 1224 - DNA, 51 bp, Homo sapiens
gcatctggaggcagagttctcactamaacctttccaagagctgtttcaatt

SEQ ID NO: 1225 - DNA, 51 bp, Homo sapiens
ttgccaacaaattttggaaacctcayggctgaatttttctcgtctctttat

SEQ ID NO: 1226 - DNA, 51 bp, Homo sapiens
gaaaaacttgaattggagtttattgragttataagagatcttgtagaccat

SEQ ID NO: 1227 - DNA, 51 bp, Homo sapiens
cacacttgaaagtttcccagtaatasaataaagtagtggtttttaaatgta

SEQ ID NO: 1228 - DNA, 51 bp, Homo sapiens
gaatgacattactatgattgatgtgkacaatagcaattcgctccctacaat

SEQ ID NO: 1229 - DNA, 51 bp, Homo sapiens
tattggctttcagccaactattacamatgaagattaaatatgttgatttta

SEQ ID NO: 1230 - DNA, 51 bp, Homo sapiens
caacccttccatttactaactgtacrccatgcagattacttaatctcctaa

SEQ ID NO: 1231 - DNA, 51 bp, Homo sapiens
tatcaacaatgcctagttttataccsaatgaataaatgacacaatgaataa

SEQ ID NO: 1232 - DNA, 51 bp, Homo sapiens
atgtcagattagagaaaccaaacaaygtgacggtacatgtgatgaaaagct

SEQ ID NO: 1233 - DNA, 51 bp, Homo sapiens
tttcacaaaataatttcatacacaaygttccctggggaccctgctggagga

SEQ ID NO: 1234 - DNA, 51 bp, Homo sapiens
gcccaacaggcaaacatgactgcaaygtaaacagccagtactcaccatgtt

SEQ ID NO: 1235 - DNA, 51 bp, Homo sapiens
catccctcctctgacttaccggatayactacaccactttataaaatagttg

SEQ ID NO: 1236 - DNA, 51 bp, Homo sapiens
cagacacaaccagcaaggcagaggargtatgtacaatcctacattcatcaa

SEQ ID NO: 1237 - DNA, 51 bp, Homo sapiens
caatttgtttaaagtaatacatatawtagtcatggaattatcaacacaaat

SEQ ID NO: 1238 - DNA, 33 bp, Homo sapiens
tcctatactaccttatytatccacctcaccaat

SEQ ID NO: 1239 - DNA, 51 bp, Homo sapiens
gaacctgagactgaaaccaacacaayggaaagcagaactgaaatatgaaga

SEQ ID NO: 1240 - DNA, 51 bp, Homo sapiens
taggacaacatctcacaaaatcataygttacaggaaatgcatgaacagcca

SEQ ID NO: 1241 - DNA, 51 bp, Homo sapiens
cacttgaattgcttacatatttcccyatcattttgctaattacactgtggc

SEQ ID NO: 1242 - DNA, 51 bp, Homo sapiens
cctagactttggagaattgctggaaracattttagcatccaattcatcgag

SEQ ID NO: 1243 - DNA, 51 bp, Homo sapiens
tccagagaagttcctctgtaactcasgatgttattttccctagcggggaca

SEQ ID NO: 1244 - DNA, 51 bp, Homo sapiens
gtccccagtcacaacagtttttgacwcctgtgttgactcaatgaagtcaat

SEQ ID NO: 1245 - DNA, 51 bp, Homo sapiens
tttggacaactggcaaagagtttgargttagaaaaattagtgatcatacac

SEQ ID NO: 1246 - DNA, 51 bp, Homo sapiens
ctccttgttatgaaatatccttctcrcccttcaaaatatccttttctaaca

SEQ ID NO: 1247 - DNA, 51 bp, Homo sapiens
agttatacaaaaattatcttctctcrcaaaaataattagttagcaaatgtg

SEQ ID NO: 1248 - DNA, 51 bp, Homo sapiens
agtagaattcaagttcacagaatacycaaactaaggtgagtgatcagacag

SEQ ID NO: 1249 - DNA, 51 bp, Homo sapiens
tcatgacaaatttatctttcaactartaaactagggcctcctcagcaaaag

SEQ ID NO: 1250 - DNA, 51 bp, Homo sapiens
ttcaatgtaaccacacatttctccayggtcttccgcaatgccatggtctcc

SEQ ID NO: 1251 - DNA, 51 bp, Homo sapiens
cctggcaatacttttaatcaagctasggtagaataaacctccatatcacca

SEQ ID NO: 1252 - DNA, 51 bp, Homo sapiens
aaatctaaattataatatggtagtaygaaacatctgtatatccttaaatat

SEQ ID NO: 1253 - DNA, 51 bp, Homo sapiens
tacaggaatgattactgcaaaggaayggtaatcaagaaataaatatttagc

SEQ ID NO: 1254 - DNA, 51 bp, Homo sapiens
ctaccctgtgcaatgtcatgatgatmaggaggccttttctaccacacacac

SEQ ID NO: 1255 - DNA, 51 bp, Homo sapiens
tttaaaatcaagtaatcaagagaaaygaggacgttgacattgctttataat

SEQ ID NO: 1256 - DNA, 51 bp, Homo sapiens
caagggtctatcggaagtttaaagcmatgcagcgggaagtaccttgccttt

SEQ ID NO: 1257 - DNA, 51 bp, Homo sapiens
caagcggaccttaaaatctccatctsactttgacactatcacgaagtgtgc

SEQ ID NO: 1258 - DNA, 51 bp, Homo sapiens
tgctctcttgatcttttatatcacawcttctgtgtagggatttgcacagct

SEQ ID NO: 1259 - DNA, 51 bp, Homo sapiens
acaatttgccttaactaatttctatracaaacaatgcctatgggaatgaac

SEQ ID NO: 1260 - DNA, 51 bp, Homo sapiens
atttctgataaatgtatccactctaygaaattagaaaaatgtgtgctgaaa

SEQ ID NO: 1261 - DNA, 51 bp, Homo sapiens
attcctagtccagctttggaattccyatgtggatttaggtgaactgcccct

SEQ ID NO: 1262 - DNA, 51 bp, Homo sapiens
acatgtctctgatagaacaatgtgayggaataacaccaatggaggaactga

SEQ ID NO: 1263 - DNA, 51 bp, Homo sapiens
gaggggggcttgcccaaatttgtgayagaaatctgccatgagctgaggagt

SEQ ID NO: 1264 - DNA, 51 bp, Homo sapiens
cttccaacaatacgttggcttggaamcattagctgtaataaaggaggttac

SEQ ID NO: 1265 - DNA, 51 bp, Homo sapiens
taaagtgttatattgaaactcaataygcaaaacaaaagcaatgcccctctt

SEQ ID NO: 1266 - DNA, 51 bp, Homo sapiens
ttccctcaccaattcttcatgcatgyaaagtcatctttataaagcacagac

SEQ ID NO: 1267 - DNA, 51 bp, Homo sapiens
atatgtgtgtgtgtgtgtttgtgtayatactcttgtcaaattctaattaac

SEQ ID NO: 1268 - DNA, 51 bp, Homo sapiens
agatatgaaatgcctattgtgaccaytagatataaagatgacaaaaaattt

SEQ ID NO: 1269 - DNA, 51 bp, Homo sapiens
ccttccacaatactctcaagaaaacrccggccgggcacggtgcctcatgcc

SEQ ID NO: 1270 - DNA, 51 bp, Homo sapiens
ttctccaattctgttcaatagtaaayggctaaaatggaccttatttgagtt

SEQ ID NO: 1271 - DNA, 51 bp, Homo sapiens
gaaaatattgtagaaatcagtcttayggtctcagttgggaagggaagactt

SEQ ID NO: 1272 - DNA, 51 bp, Homo sapiens
aaatgatgtgactatttttggacttyaacttcctcatctattgtcaaaaat

SEQ ID NO: 1273 - DNA, 51 bp, Homo sapiens
cattttggactagcagaagcatctcraatccggacatacagagtcacagga

SEQ ID NO: 1274 - DNA, 51 bp, Homo sapiens
atgttttgactaagttagacaatctmaaaaagcatatatggtggagaggca

SEQ ID NO: 1275 - DNA, 51 bp, Homo sapiens
atgattacttagttttatgggaaaayactccagaggacacgctctttggtc

SEQ ID NO: 1276 - DNA, 51 bp, Homo sapiens
actacatttcgaagacaagtcaggckgatttctagattaaattcccctgtc

SEQ ID NO: 1277 - DNA, 51 bp, Homo sapiens
atatcattcatagtgacaaatatacwcaagaactatcagggacaaatttga

SEQ ID NO: 1278 - DNA, 51 bp, Homo sapiens
caatttgtacaaggtctgatatttcwcaggttttgcatagaagcaaaggca

SEQ ID NO: 1279 - DNA, 51 bp, Homo sapiens
taagctaaattccctctgttggttamaataaaaataaacataatctgatct

SEQ ID NO: 1280 - DNA, 51 bp, Homo sapiens
gaagaggaattagaagaaagtgagcrcaatcgatgaacacagatcaactgg

SEQ ID NO: 1281 - DNA, 51 bp, Homo sapiens
aaaaactaaatgaaagcgcaaatccrgagacgtaagttgaccactgaagct

SEQ ID NO: 1282 - DNA, 51 bp, Homo sapiens
agtccttttgcttaagatttcatcakcctggtaatctgtagatttcctaat

SEQ ID NO: 1283 - DNA, 51 bp, Homo sapiens
agaagacactactattttcaaatcamctgtcaaatgaataactttggtatc

SEQ ID NO: 1284 - DNA, 51 bp, Homo sapiens
attcctatcaatatgcatcagattayacgaaaacacaagttgaccgatagt

SEQ ID NO: 1285 - DNA, 51 bp, Homo sapiens
atttcaggagtgtaaagaatcttgaygtcaccttatggaaaggggatgaaa

SEQ ID NO: 1286 - DNA, 51 bp, Homo sapiens
gaccaggaccaacatcgatttctaamgtcctgcagctcccgaccacagaca

SEQ ID NO: 1287 - DNA, 51 bp, Homo sapiens
gaatactccaacatgcatcaatcaarcacataaagaatcaatcatggagga

SEQ ID NO: 1288 - DNA, 51 bp, Homo sapiens
gaggagtgtttgtgtgtcagacttaygctggtgttgaggccctagaaaggt

SEQ ID NO: 1289 - DNA, 51 bp, Homo sapiens
agttttttaaaccatgatggactacrgtaacaataaattgcattttttagt

SEQ ID NO: 1290 - DNA, 51 bp, Homo sapiens
cacagctgcagtggaaatgtaacacyatccttcctctttgctaagggtttt

SEQ ID NO: 1291 - DNA, 51 bp, Homo sapiens
aaataatttcctaagaacataaatawctaaagacaaatgagcatatcacaa

SEQ ID NO: 1292 - DNA, 51 bp, Homo sapiens
ccttctgagcagaaaaatttcttacrcatcttatacgggttgtgcatgcct

SEQ ID NO: 1293 - DNA, 51 bp, Homo sapiens
cacaatccaaagcctaattatgatgmattacaataatcaaactcttttttg

SEQ ID NO: 1294 - DNA, 51 bp, Homo sapiens
tggggggaaaaaaacccacagtgackattgtgtttcctatcattctttttt

SEQ ID NO: 1295 - DNA, 51 bp, Homo sapiens
aaaattggggagtaggcacattgaartcttgcgtcctagtggtgactaatc

SEQ ID NO: 1296 - DNA, 51 bp, Homo sapiens
gctttattacgttgagtccagagtaktacgttctgtagtgctagtttagcg

SEQ ID NO: 1297 - DNA, 51 bp, Homo sapiens
catggccatgttagcacttataacaytgtaatttttgctagactctaggtt

SEQ ID NO: 1298 - DNA, 51 bp, Homo sapiens
cagcctgaaacaaaccaatttaagaygcatgaatgagaaacaagattttat

SEQ ID NO: 1299 - DNA, 51 bp, Homo sapiens
agtgaataattcataaatatgacaawgatcttcatggtgaaataaaagtaa

SEQ ID NO: 1300 - DNA, 51 bp, Homo sapiens
gcattcaaaaatgcaattaggaaaaytcgtacacgtgtctctggattggtt

SEQ ID NO: 1301 - DNA, 51 bp, Homo sapiens
attcattaccaccttcacctaaaccrcattttattccacttggaagcaagg

SEQ ID NO: 1302 - DNA, 51 bp, Homo sapiens
catccatgatgagctagtttctctamaatgcatgcttagtttaaatcctat

SEQ ID NO: 1303 - DNA, 51 bp, Homo sapiens
aaagagaagggaacttaatcattacrgttgtggggaatgttactggggcca

SEQ ID NO: 1304 - DNA, 51 bp, Homo sapiens
ggactccaccaccattgcaaagccasacaagtttaagttttttggggtatt

SEQ ID NO: 1305 - DNA, 51 bp, Homo sapiens
gacctgcagaataggaattgaaatayctgattgcacattcacacaggcaat

SEQ ID NO: 1306 - DNA, 51 bp, Homo sapiens
tcaagttaatgttctggttaatttaygctgattcctctaaagtcacacatc

SEQ ID NO: 1307 - DNA, 51 bp, Homo sapiens
atacattttattgaagactttggaayaacggtgaggtctcgttgatgacag

SEQ ID NO: 1308 - DNA, 51 bp, Homo sapiens
ggtttgccactaactacagtgacaaygaggaaactagaggatagaggtcct

SEQ ID NO: 1309 - DNA, 51 bp, Homo sapiens
tagagccctgaatgacttttctgagratttaatctagttgcatagctattt

SEQ ID NO: 1310 - DNA, 51 bp, Homo sapiens
tgattattttaataggctgtactaawccattttatatttgaaataaataat

SEQ ID NO: 1311 - DNA, 51 bp, Homo sapiens
actctatagttaattctttcacgaamccattatttggtaacatagttaacc

SEQ ID NO: 1312 - DNA, 51 bp, Homo sapiens
tttcatttcccctctccttgtaagcrcacttgaaaggaaggaaggtgcatg

SEQ ID NO: 1313 - DNA, 51 bp, Homo sapiens
ccagggtcaccatggttctagtaaaygcacggtgtaattctctgaaatgtg

SEQ ID NO: 1314 - DNA, 51 bp, Homo sapiens
aagcaattgtttggcttttattcacyggggttttctatgtcagtgctgatt

SEQ ID NO: 1315 - DNA, 51 bp, Homo sapiens
catatggtcactctatttgataacartaatctgtgatgactatgttaatct

SEQ ID NO: 1316 - DNA, 51 bp, Homo sapiens
taagaaagaagaattagcagattaarccttaataatatataaatactttac

SEQ ID NO: 1317 - DNA, 51 bp, Homo sapiens
aagagacatttgacaacctaatatcratggcttgaacaatggctttactaa

SEQ ID NO: 1318 - DNA, 51 bp, Homo sapiens
tggaggaacaccatcatatagaggakattcgatcaaagcaaaggatttttc

SEQ ID NO: 1319 - DNA, 51 bp, Homo sapiens
cctttggggaccatgactccatggayaggactgtcattcagaaataccaca

SEQ ID NO: 1320 - DNA, 51 bp, Homo sapiens
tggtcacttcctcttcatcccaggakattgcattctcagcacattctacag

SEQ ID NO: 1321 - DNA, 51 bp, Homo sapiens
taatgttgtctaaagaacgaagaagrtggcaaagatttgtcaggaaggaga

SEQ ID NO: 1322 - DNA, 51 bp, Homo sapiens
ggaaaccttttcgtcattttcttaaygtggacagataattgtctccaaacc

SEQ ID NO: 1323 - DNA, 51 bp, Homo sapiens
gggtttgactatcccccaaaatcaamgtttttcactggtttgagtctacta

SEQ ID NO: 1324 - DNA, 51 bp, Homo sapiens
aaatacatgtaatgtaaagttgatcrttttaaacaccttacagcgtgcaat

SEQ ID NO: 1325 - DNA, 51 bp, Homo sapiens
tccatgtaaattctgtcattaaaaaygatgccagaagtgtggtagttgaga

SEQ ID NO: 1326 - DNA, 51 bp, Homo sapiens
ccaaggactctgtctttgaccacagsctggcacaggagttaaattcaagcc

SEQ ID NO: 1327 - DNA, 51 bp, Homo sapiens
gctttttaagtcacaggaagaaaacyagacttagagaaggacaatcctttt

SEQ ID NO: 1328 - DNA, 51 bp, Homo sapiens
agctggagagctgacttagctgccargatgtcaatgaagccgaaaactcct

SEQ ID NO: 1329 - DNA, 51 bp, Homo sapiens
tggagttgggtcttcatgtcacctaktcagaagttcatatcatctttctga

SEQ ID NO: 1330 - DNA, 51 bp, Homo sapiens
gtaatgtggcaaactggttctacacrcagttctggaagcaagaatgttaaa

SEQ ID NO: 1331 - DNA, 51 bp, Homo sapiens
ctagaggtcagaagtctgaaatcaakgtgtaaagagggccctactccctcc

SEQ ID NO: 1332 - DNA, 51 bp, Homo sapiens
acatatccatctcatctccacaattkttgctttaggaattcctcaattttt

SEQ ID NO: 1333 - DNA, 51 bp, Homo sapiens
taagagatctgatacaatctttagaracttaaattcttcattaaactcagt

SEQ ID NO: 1334 - DNA, 51 bp, Homo sapiens
gaagtaggtgttcactttaaacttayggcttgatctttttctaccctctgt

SEQ ID NO: 1335 - DNA, 51 bp, Homo sapiens
acattattttttccataaaaactatmagaccatttcattcaatttaaaaaa

SEQ ID NO: 1336 - DNA, 51 bp, Homo sapiens
ctccttaagaaatgtggtattaaacygatatatatttttaatgtaactttt

SEQ ID NO: 1337 - DNA, 51 bp, Homo sapiens
aggaaagcctagaatgtgtttgtcaycaagatcaaaaggtgaaagagtttc

SEQ ID NO: 1338 - DNA, 51 bp, Homo sapiens
cctctactcttttctttctgtttccwaatgatacactcctttcttgaactc

SEQ ID NO: 1339 - DNA, 51 bp, Homo sapiens
taactgtgcacacacacagctacgawtatgtatatcaatatttgcataaga

SEQ ID NO: 1340 - DNA, 51 bp, Homo sapiens
taatgagacttctgctatgtccactrgaagaagtattcccctttgggtacc

SEQ ID NO: 1341 - DNA, 51 bp, Homo sapiens
aatagacattccttttgtatttggaraagataactgttgggtactgagctt

SEQ ID NO: 1342 - DNA, 51 bp, Homo sapiens
atcacaaccagtcctttaagtcttasctattatcttgcagactatattctg

SEQ ID NO: 1343 - DNA, 51 bp, Homo sapiens
tttggctacatttatattcaagtgarctaacggcactggtccaaaatttct

SEQ ID NO: 1344 - DNA, 51 bp, Homo sapiens
ataatagtgataaggaaagcaaaaaygacaaaggagatcattgatggggga

SEQ ID NO: 1345 - DNA, 51 bp, Homo sapiens
acatgtattatcttcccacgatcagyttcaagaaccttcatgattctgtca

SEQ ID NO: 1346 - DNA, 51 bp, Homo sapiens
agtagctaccatattccgtaacgcargtagagtacactcagagtgagaaag

SEQ ID NO: 1347 - DNA, 51 bp, Homo sapiens
agaagcaataaattataatggcttamacttatattatgctttcccagatga

SEQ ID NO: 1348 - DNA, 51 bp, Homo sapiens
cttcttttttcatgttggatgagaartagagaaaagggatgatttgtgaag

SEQ ID NO: 1349 - DNA, 51 bp, Homo sapiens
tgggccagaggaaaatttgactaacrgatatcacaccaaaaacctgctctc

SEQ ID NO: 1350 - DNA, 51 bp, Homo sapiens
ctaaaccttgtagacaagtgagtcayctgatatgtatagaagctgtgatat

SEQ ID NO: 1351 - DNA, 51 bp, Homo sapiens
acatttctggtgttagcatcaatatkaaacatagcctctgataaatcataa

SEQ ID NO: 1352 - DNA, 33 bp, Homo sapiens
tcctctctcaagtcaaygtttgattggtgtgtg

SEQ ID NO: 1353 - DNA, 51 bp, Homo sapiens
attctttcaaatgatcaaatattccrtgagtgttccaattaatttatgtat

SEQ ID NO: 1354 - DNA, 51 bp, Homo sapiens
ctttgctttttagatgaaaaatttargctaagacaaataactaacttatca

SEQ ID NO: 1355 - DNA, 51 bp, Homo sapiens
ggaaataaagcttatttttctgtaaytaatatagtcttcttcatgagcctc

SEQ ID NO: 1356 - DNA, 51 bp, Homo sapiens
atcctagatcaaaatcttgagtatasagctccagttttcagctttgattac

SEQ ID NO: 1357 - DNA, 51 bp, Homo sapiens
cccttgctaccactaagtaatagtaygctaaaatcataagggcaggccgag

SEQ ID NO: 1358 - DNA, 51 bp, Homo sapiens
tgagttacctgacagacttccaaaasatcccctgtgaggttggatcaggaa

SEQ ID NO: 1359 - DNA, 51 bp, Homo sapiens
attccaaggtaatgacagaattttaygttcctcagaagctgatccttgtac

SEQ ID NO: 1360 - DNA, 51 bp, Homo sapiens
cctgctgaggagaggaccttgataamagagtacatattcaggaatgacagt

SEQ ID NO: 1361 - DNA, 51 bp, Homo sapiens
tcagaacaaagaatacccattagagragttttgctcaggcaggaatggcct

SEQ ID NO: 1362 - DNA, 51 bp, Homo sapiens
agttttcttgtacaaacggttgttcyactctttctagaccaaagatcattt

SEQ ID NO: 1363 - DNA, 33 bp, Homo sapiens
ccaaagtgctggaattrtaaatgtgagccacca

SEQ ID NO: 1364 - DNA, 51 bp, Homo sapiens
cctggtttcagactttgttgtgaacmcagatttcatttcaagttctgacag

SEQ ID NO: 1365 - DNA, 51 bp, Homo sapiens
atattgttttcagtcaggtttaggamaagtaggacaaacacaactttccca

SEQ ID NO: 1366 - DNA, 51 bp, Homo sapiens
caaaatggtatgtttaatcttatttwacaactgtgaaaactgagctgagag

SEQ ID NO: 1367 - DNA, 51 bp, Homo sapiens
tggtgggaagtttaataaacactctsaagctattaaaaggtttctctacga

SEQ ID NO: 1368 - DNA, 51 bp, Homo sapiens
acttgctgagcttttaaccgagacayagaaattaggtaaagatattaagta

(19) United States
(12) Patent Application Publication
(10) Pub. No.: US 2006/0110751 A1
(43) Pub. Date: May 25, 2006

(54) Method and kit for detecting a risk of essential arterial hypertension

(75) Inventors: Jukka T. Salonen, Kuopio (FI); Mia Pirskanen, Kuopio (FI); Pekka Uimari, Kuopio (FI); Ricardo Fuentes, Siilinjarvi (FI); Outi Kontkanen, Kuopio (FI); Juha-Matti Aalto, Siilinjarvi (FI)

Correspondence Address: BIRCH STEWART KOLASCH & BIRCH, PO BOX 747, FALLS CHURCH, VA, 22040-0747, US

(73) Assignee: Oy Jurilab Ltd

(21) Appl. No.: 11/245,248

(22) Filed: Oct. 7, 2005

(30) Foreign Application Priority Data
Nov. 19, 2004 (FI) 20041490

(51) Int. Cl. C12Q 1/68; G06F 19/00

(52) U.S. Cl. 435/6; 705/3

(57) ABSTRACT

Genes, SNP markers and haplotypes of susceptibility or predisposition to hypertension (HT) are disclosed. Methods for diagnosis, prediction of clinical course and efficacy of treatments for HT using polymorphisms in the HT risk genes are also disclosed. The genes, gene products and agents of the invention are also useful for monitoring the effectiveness of prevention and treatment of HT. Kits are also provided for the diagnosis, selecting treatment and assessing prognosis of HT.

### COMPACT DISK

Pursuant to 37 C.F.R. § 1.52(e), a compact disc containing an electronic version of the uence Listing in lieu of a paper copy of the Sequence Listing has been submitted as a part of the present application. The compact disc also includes data tables in landscape format. A second compact disc is submitted and is an identical copy of the first compact disc. The discs are labeled “Copy 1” and “Copy 2,” respectively, and each disc contains the following files:

| File Name | Create Date | File Size |
|---|---|---|
| Sequence listing.txt | Aug. 8, 2005 | 199 KB |
| Table2_HT.txt | Aug. 10, 2005 | 37 KB |
| Table3_HT.txt | Aug. 9, 2005 | 56 KB |
| Table4_HT.txt | Aug. 9, 2005 | 68 KB |
| Table5_HT.txt | Aug. 9, 2005 | 7 KB |
| Table6_HT.txt | Aug. 9, 2005 | 30 KB |
| Table7_HT.txt | Aug. 9, 2005 | 4 KB |
| Table8_HT.txt | Aug. 9, 2005 | 4 KB |
| Table9_HT.txt | Aug. 9, 2005 | 2 KB |
| Table10_HT.txt | Aug. 9, 2005 | 3 KB |
| Table11_HT.txt | Aug. 9, 2005 | 3 KB |

The present application hereby incorporates by reference in its entirety the material in each of the files listed above.

### BACKGROUND OF THE INVENTION

1. Field of the Invention

The present invention relates generally to the field of diagnosis of cardiovascular diseases (CVD) such as arterial hypertension (HT). More particularly, it provides a method of diagnosing or detecting a predisposition or propensity or susceptibility for HT. Specifically, the invention focuses on a method that comprises the steps of providing a biological sample from the subject to be tested and detecting the presence or absence of one or several genomic single nucleotide polymorphism (SNP) markers in the biological sample. Furthermore, the invention utilizes both genetic and phenotypic information as well as information obtained by questionnaires to construct a score that provides the probability of developing HT. In addition, the invention provides a kit to perform the method. The kit can be used to set an etiology-based diagnosis of HT for targeting of treatment and preventive interventions such as dietary advice, as well as stratification of the subject in clinical trials testing drugs and other interventions.

2. Description of Related Art

Public Health Significance of CVD and HT

Cardiovascular Diseases (CVD) (ICD/10 codes I00-I99, Q20-Q28) include ischemic (coronary) heart disease (IHD, CHD), hypertensive diseases, cerebrovascular disease (stroke) and rheumatic fever/rheumatic heart disease, among others (AHA, 2004). HT (ICD/10 I10-I15) is defined as systolic pressure of 140 mm Hg or higher, or diastolic pressure of 90 mm Hg or higher, or taking antihypertensive medicine (AHA, 2004). Apart from being a CVD itself, HT is a risk factor for other CVD, such as IHD, stroke and congestive heart failure (CHF). About half of those people who have a first heart attack and two thirds of those who have a first stroke, have blood pressure (BP) higher than 160/95 mm Hg. HT precedes the development of CHF in 91% of cases (AHA, 2004).

Of patients with HT, 90-95% have essential HT in which the underlying cause remains unknown. Essential HT refers to a lasting increase in BP with heterogeneous genetic and environmental causes. Its prevalence rises with age irrespective of the type of BP measurement and the operational thresholds used for diagnosis. HT aggregates with other cardiovascular risk factors such as abdominal obesity, dyslipidaemia, glucose intolerance, hyperinsulinaemia and hyperuricaemia, possibly because of a common underlying cause (Salonen J T et al, 1981, 1998, Staessen J A et al, 2003).

In 2001 an estimated 16.6 million—or one-third of total global deaths—resulted from the various forms of CVD (7.2 million due to HT, 5.5 million to cerebrovascular disease, and an additional 3.9 million to hypertensive and other heart conditions). At least 20 million people survive heart attacks and strokes every year, a significant proportion of them requiring costly clinical care, putting a huge burden on long-term care resources. It is necessary to recognize that CVDs are devastating to men, women and children (AHA, 2004).

Around 80% of all CVD deaths worldwide took place in developing, low and middle-income countries. It is estimated that by 2010, CVD will be the leading cause of death in both developed and developing countries. The rise in CVDs reflects a significant change in dietary habits, physical activity levels, and tobacco consumption worldwide as a result of industrialization, urbanization, economic development and food market globalization (WHO, 2004). This emphasizes the role of relatively modem environmental or behavioral risk factors. However, ethnic differences in the incidence and prevalence of CVD and the enrichment of CVD in families suggest that heritable risk factors play a major role.

In terms of disability measured in disability-adjusted life years (DALYs) CVD caused 9.7% of global DALYs, 20.4% of DALYs in developed countries and 8.3% of DALYs in the developing countries. HT caused 1.4% of global DALYs, 4.7% of DALYs in developed countries and 0.9% of DALYs in the developing countries (Murray C J L and Lopez A D, 1997).

On the basis of data from the NHANES III study (1988-1994), it is estimated that in 2001, 64.4 million Americans were affected by some form of CVD, which corresponds to a prevalence of 22.6% (21.5% for males, 22.4% for females). Of these, 50 million had HT (20% prevalence). Of those with HT, 30% do not know they have HT; 34% are on medication and have HT controlled; 25% are on medication but do not have their HT under control; and 11% are not on medication (AHA, 2004). HT is also a public health problem in developing countries where prevalences of 10% or higher are common and it is frequently associated with low levels of awareness, treatment and control (Fuentes R M et al, 2000).

The cost of CVD in the United States in 2004 was estimated at $368.4 billion ($133.2 billion for HT, $53.6 billion for stroke, $55.5 billion for hypertensive disease). This figure includes health expenditures (direct costs) and lost productivity resulting from morbidity and mortality (indirect costs) (AHA, 2004).

Pathophysiology of Essential HT

The pressure required to move blood through the circulatory bed is provided by the pumping action of the heart [cardiac output (CO)] and the tone of the arteries [peripheral resistance (PR)]. Each of these primary determinants of BP is, in turn, determined by the interaction of a complex series of factors.

Factors Affecting Cardiac Output

An increased CO has been found in some young, borderline hypertensives who may display a hyperkinetic circulation. If it is responsible for HT, the increase in CO could logically arise in two ways: either from an increase in fluid volume (preload) or from an increase in contractility from neural stimulation of the heart. However, even if it is involved in the initiation of HT, the increased CO probably does not persist. The typical hemodynamic finding in established HT is an elevated PR and normal CO (Cowley A W, 1992).

Although an increased heart rate may not simply be a reflection of a hyperdynamic circulation or an indicator of increased sympathetic activity, multiple epidemiologic surveys have shown that an elevated heart rate is an independent predictor of the development of HT (Palatini P and Julius S, 1999).

Left ventricular hypertrophy has generally been considered a compensatory mechanism to an increased vascular resistance. However, it could also reflect a primary response to repeated neural stimulation and, thereby, could be an initiating mechanism for HT (Julius S et al., 1991c) as well as an amplifier of CO that reinforces the elevation of BP from arterial stiffening (Segers P et al., 2000).

Another mechanism that could induce HT by increasing CO would be an increased circulating fluid volume (preload). However, in most studies, subjects with high BP have a lower blood volume and total exchangeable sodium than normal subjects (Harrap S B et al., 2000). Even without an expanded total volume, blood may be redistributed so that more is in the central or cardiopulmonary section because of greater peripheral vasoconstriction (Schobel H P et al., 1993). Venous return to the heart would thereby be increased and could mediate an increased CO.

Excess sodium intake induces HT by increasing fluid volume and preload, thereby increasing CO (Chobanian A V and Hill M, 2000). Both experimental data (Tobian L, 1991) and epidemiologic evidence (Stamler J et al., 1997) support a close association between HT and a high sodium-potassium ratio in humans. Because almost everyone in industrialized societies ingests a high-sodium diet, the fact that only about half will develop HT suggests a variable degree of BP sensitivity to sodium (Weinberger M H, 1996).

In healthy people, when BP increases, renal excretion of sodium and water increases, shrinking fluid volume and returning the BP to normal—this phenomenon is pressure-natriuresis. On the basis of animal experiments and computer models, the regulation of body fluid volume by the kidneys is considered to be the dominant mechanism for the long-term control of BP (Guyton A C 1961, 1992). Therefore, if HT develops, something must be wrong with the pressure-natriuresis control mechanism; otherwise the BP would return to normal (Cowley A W and Roman R J, 1996). In patients with primary HT a resetting of the pressure-sodium excretion curve prevents the return of BP to normal (Palmer B F, 2001). The shift in pressure-natriuresis requires increased BP to maintain fluid balance. The pressure-natriuresis relationship can be modified by neural and humoral factors including the renin-angiotensin system (RAS), sympathetic nervous activity, atrial natriuretic factor, metabolites of arachidonic acid, and intrarenal nitric oxide (Moreno C et al., 2001; Majid D S et al., 2001).

The major modifier is likely to be the RAS (Hall J E et al., 1999; van Paassen P et al., 2000), with an increase in renal sodium reabsorption occurring at concentrations of Angiotensin II much below those needed for peripheral vasoconstriction. Angiotensin II acts not only on vascular smooth muscle and the adrenal cortex but also within the heart, kidneys, and central and autonomic nervous systems. These actions amplify its volume-retaining and vasoconstrictive effects on the peripheral vascular system, thus affecting both CO and PR. Furthermore, Angiotensin II induces cell growth and hypertrophy independent of its effect on BP (Su E J et al., 1998). Moreover, Angiotensin II appears to induce an inflammatory response in vascular smooth muscle cells (Kranzhöfer R et al., 1999), with activation of nuclear factor k-B (Luft F C, 2001) and adhesion molecule-1 expression (Tummala P E et al., 1999), which may serve as direct links to atherosclerosis.

Stress may activate the sympathetic nervous system (SNS) directly; and SNS overactivity, in turn, may interact with high sodium intake, the RAS, and insulin resistance, among other possible mechanisms. Considerable evidence supports increased SNS activity in early HT (Esler M et al., 2001) and, even more impressively, in the still-normotensive offspring of hypertensive parents, of whom a large number are likely to develop HT. Whatever the specific role of SNS activity in the pathogenesis of HT, it appears to be involved in the increased cardiovascular morbidity and mortality that afflicts hypertensive patients during the early morning hours. Epinephrine levels begin to increase after awakening and norepinephrine rises sharply on standing (Dodt C et al., 1997). As a consequence of the increased SNS activity, BP rises suddenly and markedly, and this rise is at least partly responsible for the increase in sudden death, heart attack, and stroke during the early morning hours. Increased sympathetic activity is probably also responsible for the increased heart rate present in many hypertensives that was previously noted to be associated with increased cardiovascular mortality.

Factors Affecting Peripheral Resistance

HT is maintained by increased PR, largely due to decreased arterial lumen size or radius. According to Poiseuille's law, vascular resistance is positively related to both the viscosity of blood and the length of the arterial system, and negatively related to the third power of the luminal radius. Because neither viscosity nor length is altered much if at all, and because small changes in the luminal radius can have a major effect, it is apparent that the increased vascular resistance seen in established HT must reflect changes in the calibre of the small resistance arteries and arterioles (Folkow B et al., 1970). Because of the increased wall thickness-lumen diameter ratio, higher wall stress and intraluminal pressure develop when resistance vessels are stimulated.

In HT, small arteries undergo functional, structural and mechanical changes, resulting in reduced lumen size and increased peripheral resistance (Mulvany M J, 2002; Intengan HD and Schiffrin E L. 2001). Functional alterations include enhanced reactivity or impaired relaxation, and reflect changes in excitation-contraction coupling, altered electrical properties of vascular smooth muscle cells, or endothelial dysfunction (Johns D G et al, 2000; Feldman R D and Gros R, 1998). Major structural changes include remodelling due to increased cell growth, extracellular matrix deposition and inflammation (Mulvany M J, 2002; Intengan HD and Schiffrin E L, 2001; Brasier A R, 2002). Vascular smooth muscle cells are central to these events and play a fundamental role in the dynamic processes underlying the alterations that occur in HT.

Vascular changes in HT are associated with humoral and mechanical factors that modulate signalling events, resulting in abnormal function and growth of cellular components of the media (Touyz R M, 2000; Koller A, 2002). The humoral factors that regulate arteries in HT include vasoconstrictor agents such as angiotensin II, endothelin-1, catecholamines and vasopressin; vasodilator agents such as nitric oxide, endothelium-derived hyperpolarizing factor and natriuretic peptides; growth factors such as insulin-like growth factor-1, platelet-derived growth factor (PDGF), epidermal growth factor (EGF) and basic fibroblast growth factor; and cytokines such as transforming growth factor-[beta], tumour necrosis factor and interleukins (Touyz R M, 2000). Mechanical factors that influence the vasculature in HT include shear stress, wall stress and the direct actions of pressure itself (Touyz R M, 2000; Koller A, 2002). In addition to these factors, there is growing evidence that reactive oxygen species (ROS) that act as intercellular and intracellular signalling molecules, regulate vascular tone and structure (Wilcox C S, 2002; Berry C et al, 2001).

A recent advance in the field of angiotensin II signalling was the demonstration that, in addition to its vasoconstrictor properties, angiotensin II has potent mitogenic-like and proinflammatory-like characteristics. These actions are mediated through phosphorylation of both nonreceptor tyrosine kinases and receptor tyrosine kinases (Touyz R M, 2003). It is also becoming increasingly apparent that many signalling events that underlie abnormal vascular function in HT are influenced by changes in intracellular redox status. In particular, increased bioavailability of ROS stimulates growth-signalling pathways, induces expression of proinflammatory genes, alters contraction-excitation coupling and impairs endothelial function (Touyz R M, 2003).

In concert with the various functional and structural changes that are responsible for HT, the arteries become stiffer or less elastic. Vascular stiffness progressively increases with age (Slotwiner D J et al., 2001) and is responsible for the progressive increase in systolic as compared to diastolic pressure, leading to the typical increase of pulse pressure that is now recognized to be the major determinant of cardiovascular risk (Beltran A et al., 2001). Measures of stiffness and elasticity have been shown to be an independent predictor of the development of HT (Liao D et al., 1999) and a marker of cardiovascular risk in those with HT (Blacher J et al., 1999). Changes in the physical characteristics of the large arteries reflected in the BP pulse contour alter not only BP and pulse pressure, but also cardiac work and performance.

The complexity of pathophysiologic mechanisms that lead to BP elevation is such that selective, mechanistically based antihypertensive treatment is rarely possible in any hypertensive patient. HT is highly prevalent among middle-aged and elderly persons, and the success rate in controlling BP in these individuals is poor. Current treatment guidelines generally recommend a generic approach to treating HT, with little emphasis on selecting therapy on the basis of the underlying pathophysiology of the elevated BP (Chobanian AV et al, 2003; ESH/ESC, 2003). With increased recognition of specific causes, it may be possible to develop therapies selective for distinct pathophysiologic mechanisms with fewer adverse effects, resulting in more effective BP reduction. The use of powerful new techniques of genetics, genomics, and proteomics, integrated with systems physiology and population studies, will make more selective and effective approaches to treating and even preventing HT possible in the coming decades (Oparil S et al, 2003).

Essential HT: a Polygenic Disease

Nuclear family studies show greater similarity in BP within families than between families, with heritability estimates ranging between 0.20 and 0.46 (Fuentes R M, 2003). Twin studies document greater concordance of BP in monozygotic than dizygotic twins, giving the highest heritability estimates between 0.48 and 0.64 (Fuentes R M, 2003). Adoption studies demonstrate greater concordance of BP among biological siblings than adoptive siblings living in the same household, estimating heritability between 0.45 and 0.61 (Fuentes R M, 2003).

Single genes can have major effects on BP, accounting for the rare Mendelian forms of high and low BP (Lifton R P et al, 2001). Although identifiable single-gene mutations account for only a small percentage of HT cases, studying these rare disorders may elucidate pathophysiologic mechanisms that predispose to more common forms of HT and may suggest novel therapeutic approaches (Lifton R P et al, 2001). Mutations in 10 genes that cause Mendelian forms of human HT and 9 genes that cause hypotension have been described to date (Lifton R P et al, 2001; Wilson F H et al, 2001). These mutations affect BP by altering renal salt handling, reinforcing the hypothesis that the development of HT depends on genetically determined renal dysfunction with resultant salt and water retention (Guyton A C, 1991). Importantly, all the monogenic HT syndromes identified to date are caused by defects resulting in renal salt retention, whereas all the low BP syndromes share a common mechanism of excess renal sodium loss (Hopkins P N and Hunt S C, 2003).

The best studied monogenic cause of HT is the Liddle syndrome, a rare but clinically important disorder in which constitutive activation of the epithelial sodium channel predisposes to severe, treatment-resistant HT (Shimkets R A et al, 1994). Epithelial sodium channel activation has been traced to mutations in the beta or gamma subunits of the channel, resulting in inappropriate sodium retention at the renal collecting duct level. Patients with the Liddle syndrome typically display volume-dependent, low-renin, and low-aldosterone HT.

In most cases, HT results from a complex interaction of genetic, environmental, and demographic factors. Improved techniques of genetic analysis, especially candidate gene association studies and genome wide linkage analysis (genome wide scan, GWS), have enabled a search for genes that contribute to the development of primary HT in the population.

Thus far, the candidate gene approach has provided more examples than the linkage approach of gene variants that appear to affect BP. Reasonable candidate genes to consider include genes related to physiological systems known to be involved in the control of BP and genes known to affect BP in mouse models. To date more than 80 candidate genes have been evaluated for HT (Fuentes R M, 2004, unpublished review). However, the association with HT of only three genes have been widely replicated: angiotensinogen precursor (AGT), adducin 1 (ADD1) and guanine nucleotide-binding protein, beta-3 subunit (GNB3) (Hopkins P N and Hunt S C, 2003). Gene-environment interactions affecting HT treatment have been shown between AGT, ADD1 and salt intake reduction (Hunt S C et al, 1998; Hunt S C et al, 1999; Cusi D et al, 1997), and between ADD1, GNB3 and diuretic treatment (Cusi D et al, 1997; Turner S T et al, 2001). Gene-gene interactions affecting HT risk development have been shown between ADD1 and the ACE gene I/D polymorphisms (Staessen J A et al, 2001). Lessons learned from the studies of candidate genes to date include the shortcomings that result from the limited statistical power of many studies, expected variation from one population to another, the need for better phenotyping of study subjects, the relatively small effect of the genes studied on population prevalence of HT, and the lack of sufficient certainty of consequences of any genes studied thus far to make treatment recommendations based on genotype (Hopkins P N and Hunt S C, 2003).

To date more than 30 GWS studies have been reported to identify loci for BP/HT (Fuentes R M, 2004, unpublished review). Some studies utilized families, others affected or dissimilar sibling pairs. Linked loci with at least indicative LOD scores to BP/HT have been observed on every chromosome. Perhaps most striking is the lack of consistently linked loci. Koivukoski L et al, 2004 found evidence of susceptibility regions for BP/HT on chromosomes 2p12-q22.1 and 3p14.1-q12.3 that had modest or non-significant linkage in each individual study when applying the genome-search meta-analysis method (GSMA) to nine published genome-wide scans of BP (n=5) and HT (n=4) from Caucasian populations. This may serve to illustrate the heterogeneity of human HT as well as the potential shortcomings of attempting to compare studies using different methodologies.

Opportunity for Population Genetics

Previous medical research concerning the genetic etiology of HT has been based to a large extent on retrospective case-control and family studies in humans and studies in genetically modified animals. As recognized only recently, retrospective case-control studies are prone to survival and selection biases, and they have produced a myriad of biased findings concerning a large number of candidate genes. A commonly used approach is to compare gene expression between affected and unaffected persons. Gene expression studies, which are mostly cross-sectional, cannot however separate cause and consequence. Findings from animal models concerning HT cannot be generalised to humans, as the pathophysiology in humans is unique. The unsuitability of the animal studies is the main reason why genetic epidemiologic studies are the most important means in the clarification of genetic etiologies of human diseases.

Prospective cohort studies in humans overcome these problems. Developments in GWS and sequencing technology and methods of data analysis render possible the attempt to identify liability genes in complex, multifactorial traits, and to dissect the role of genetic predisposition and environment/life style factors in these disorders with new precision. Genetic and environmental effects vary over the life span, and only longitudinal studies in genetically informative data sets permit the study of such effects. A major advantage of population genetics approaches in disease gene discovery over other methodologies is that it will yield diagnostic markers that are valid in humans.

The identification of genes causing major public health problems such as HT is now enabled by the following recent advances in molecular biology, population genetics and bioinformatics: 1. the availability of new genotyping platforms that will dramatically lower operating costs and increase throughputs; 2. the application of genome scans using dense marker maps; 3. data analysis using new powerful statistical methods testing for linkage disequilibrium using haplotype sharing analysis, and 4. the recognition that a smaller number of genetic markers than previously thought is sufficient for genome scans in genetically homogeneous populations.

Traditional GWS using microsatellite markers with linkage analyses have not been successful in finding genes causing common diseases. The failure has in part been due to too small a number of genetic markers used in GWS, and in part due to too heterogeneous study populations. With the advancements of the human genome project and genotyping technology, the first dense marker maps have recently become available for mapping the entire human genome. The microarrays used by Jurilab include probes for over 100,000 single nucleotide polymorphism (SNP) markers. These SNPs form a marker map covering, for the first time, the entire genome tightly enough for the discovery of most disease genes causing HT.

Genetic Homogeneity of the East Finland Founder Population

Finns descend from two human immigration waves occurring about 4,000 and 2,000 years ago. Both Y-chromosomal haplotypes and mitochondrial sequences show low genetic diversity among Finns compared with other European populations and confirm the long-standing isolation of Finland (Sajantila A et al, 1996). During King Gustavus of Vasa (1523-1560) over 400 years ago, internal migrations created regional subisolates, the late settlements (Peltonen L et al, 1999). The most isolated of these are the East Finns.

The East Finnish population is the most genetically-homogenous population isolate known that is large enough for effective gene discovery program. The reasons for homogeneity are: the young age of the population (fewer generations); the small number of founders; long-term geographical isolation; and population bottlenecks because of wars, famine and fatal disease epidemics.

Owing to the genetic homogeneity of the East Finland population, there are fewer mutations in important disease predisposing genes and the affected individuals share a similar genetic background. Because of the stronger linkage disequilibrium (LD), fewer SNPs and fewer subjects are needed for GWS than in other populations.

### SUMMARY OF THE INVENTION

The present invention relates to single nucleotide polymorphism (SNP) markers, combinations of such markers and haplotypes associated with altered risk of HT, and genes associated with HT within or close to which said markers or haplotypes are located. Said SNP markers may be associated either with increased HT risk or reduced HT risk i.e. protective of HT. The “prediction” or risk implies here that the risk is either increased or reduced.

Thus, the present invention provides individual SNP markers associated with HT and combinations of SNP markers and haplotypes in genetic regions associated with HT, genes previously known in the art, but not known to be associated with HT, methods of estimating susceptibility or predisposition of an individual to HT, as well as methods for prediction of clinical course and efficacy of treatments for HT using polymorphisms in the HT risk genes. Accordingly, the present invention provides novel methods and compositions based on the disclosed HT associated SNP markers, combinations of SNP markers, haplotypes and genes.

The invention further relates to a method for estimating susceptibility or predisposition of an individual to HT comprising the detection of the presence of SNP markers and haplotypes, or an alteration in expression of an HT risk gene set forth in tables 2 through 8, as well as alterations in the polypeptides encoded by the said HT risk genes. The alterations may be quantitative, qualitative, or both.

The invention yet further relates to a method for estimating susceptibility or predisposition of an individual to HT. The method for estimating susceptibility or predisposition of an individual to HT is comprised of detecting the presence of at-risk haplotypes in an individual's nucleic acid.

The invention further relates to a kit for estimating susceptibility to HT in an individual comprising wholly or in part: amplification reagents for amplifying nucleic acid fragments containing SNP markers, detection reagents for genotyping SNP markers and interpretation software for data analysis and risk assessment.

In one aspect, the invention relates to methods of diagnosing a predisposition to HT. The methods of diagnosing a predisposition to HT in an individual include detecting the presence of SNP markers predicting HT, as well as detecting alterations in expression of genes which are associated with said markers. The alterations in expression can be quantitative, qualitative, or both.

A further object of the present invention is a method of identifying the risk of HT by detecting SNP markers in a biological sample of the subject. The information obtained from this method can be combined with other information concerning an individual, e.g. results from blood measurements, clinical examination and questionnaires. The blood measurements include but are not restricted to the determination of plasma or serum cholesterol and high-density lipoprotein cholesterol. The information to be collected by questionnaire includes information concerning gender, age, family and medical history such as the family history of HT and diabetes. Clinical information collected by examination includes e.g. information concerning height, weight, hip and waist circumference, systolic and diastolic BP, and heart rate.

The methods of the invention allow the accurate diagnosis of HT at or before disease onset, thus reducing or minimizing the debilitating effects of HT. The method can be applied in persons who are free of clinical symptoms and signs of HT, in those who already have clinical HT, in those who have a family history of HT, or in those who have an elevated level or levels of risk factors of HT.

The invention further provides a method of diagnosing susceptibility to HT in an individual. This method comprises screening for at-risk haplotypes that predict HT that are more frequently present in an individual susceptible to HT, compared to the frequency of its presence in the general population, wherein the presence of an at-risk haplotype is indicative of a susceptibility to HT. The “at-risk haplotype” may also be associated with a reduced rather than increased risk of HT. An “at-risk haplotype” is intended to embrace one or a combination of haplotypes described herein over the markers that show high correlation to HT. Kits for diagnosing susceptibility to HT in an individual are also disclosed.

Those skilled in the art will readily recognize that the analysis of the nucleotides present in one or several of the SNP markers of this invention in an individual's nucleic acid can be done by any method or technique capable of determining nucleotides present in a polymorphic site. As it is obvious in the art the nucleotides present in SNP markers can be determined from either nucleic acid strand or from both strands.

The major application of the current invention involves prediction of those at higher risk of developing HT. Diagnostic tests that define genetic factors contributing to HT might be used together with or independent of the known clinical risk factors to define an individual's risk relative to the general population. Better means for identifying those individuals at risk of HT should lead to better preventive and treatment regimens, including more aggressive management of the current clinical risk factors for sequelae of HT such as cigarette smoking, hypercholesterolemia, elevated LDL cholesterol, low HDL cholesterol, HT and elevated BP, diabetes mellitus, glucose intolerance, insulin resistance and the metabolic syndrome, obesity, lack of physical activity, and inflammatory components as reflected by increased C-reactive protein levels or other inflammatory markers. Information on genetic risk may be used by physicians to help convince particular patients to adjust life style (e.g. to stop smoking, reduce caloric intake, to increase exercise). Finally, preventive measures aimed at lowering blood pressure such as reduction of weight, intake of salt and alcohol, can be both better motivated to the patients and selected on the basis of the molecular subdiagnosis of HT.

A further object of the invention is to provide a method for the selection of human subjects for studies testing antihypertensive effects of drugs.

Another object of the invention is a method for the selection of subjects for clinical trials testing antihypertensive drugs.

Still another object of the invention is to provide a method for prediction of clinical course and efficacy of treatments for HT using polymorphisms in the HT risk genes. The genes, gene products and agents of the invention are also useful for treating HT, for monitoring the effectiveness of the treatment, and for drug development. Kits are also provided for the diagnosis, treatment and prognosis of HT.

### DETAILED DESCRIPTION OF THE INVENTION

Representative Target Population

An individual at risk of HT is an individual who has at least one risk factor of HT, such as family history of HT, central or other type of obesity, lack of physical activity, high sodium intake, high intake of saturated fats, low intake of potassium and/or magnesium, low HDL cholesterol, diabetes mellitus, glucose intolerance, insulin resistance and the metabolic syndrome, elevated inflammatory marker, and an at-risk allele or haplotype with one or several HT risk SNP markers.

In another embodiment of the invention, an individual who is at risk of HT is an individual who has a risk-increasing allele in an HT risk gene, in which the presence of the polymorphism is indicative of a susceptibility to HT. The term “gene,” as used herein, refers to an entirety containing all regulatory elements located both upstream and downstream as well as within of a polypeptide encoding sequence, 5′ and 3′ untranslated regions of mRNA and the entire polypeptide encoding sequence including all exon and intron sequences (also alternatively spliced exons and introns) of a gene.

Assessment for At-Risk Alleles and At-Risk Haplotypes

The genetic markers are particular “alleles” at “polymorphic sites” associated with HT. A nucleotide position at which more than one sequence is possible in a population, is referred to herein as a “polymorphic site”. Where a polymorphic site is a single nucleotide in length, the site is referred to as a SNP. For example, if at a particular chromosomal location, one member of a population has an adenine and another member of the population has a thymine at the same position, then this position is a polymorphic site, and, more specifically, the polymorphic site is a SNP. Polymorphic sites may be several nucleotides in length due to insertions, deletions, conversions or translocations. Each version of the sequence with respect to the polymorphic site is referred to herein as an “allele” of the polymorphic site. Thus, in the previous example, the SNP allows for both an adenine allele and a thymine allele.

Typically, a reference nucleotide sequence is referred to for a particular gene. Alleles that differ from the reference are referred to as “variant” alleles. The polypeptide encoded by the reference nucleotide sequence is the “reference” polypeptide with a particular reference amino acid sequence, and polypeptides encoded by variant alleles are referred to as “variant” polypeptides with variant amino acid sequences.

Nucleotide sequence variants can result in changes affecting the properties of a polypeptide. These sequence differences, when compared to a reference nucleotide sequence, include insertions, deletions, conversions and substitutions: e.g. an insertion, a deletion or a conversion may result in a frame shift generating an altered polypeptide; a substitution of at least one nucleotide may result in a premature stop codon, aminoacid change or abnormal mRNA splicing; the deletion of several nucleotides, resulting in a deletion of one or more amino acids encoded by the nucleotides; the insertion of several nucleotides, such as by unequal recombination or gene conversion, resulting in an interruption of the coding sequence of a reading frame; duplication of all or a part of a sequence; transposition; or a rearrangement of a nucleotide sequence, as described in detail above. Such sequence changes alter the polypeptide encoded by an HT susceptibility gene. For example, a nucleotide change in a gene resulting in a change in corresponding polypeptide aminoacid sequence can alter the physiological properties of a polypeptide resulting in a polypeptide having altered biological activity/function, distribution or stability.

Alternatively, nucleotide sequence variants can result in changes affecting transcription of a gene or translation of it's mRNA. A polymorphic site located in a regulatory region of a gene may result in altered transcription of a gene e.g. due to altered tissue specifity, altered transcription rate or altered response to transcription factors. A polymorphic site located in a region corresponding to the mRNA of a gene may result in altered translation of the mRNA e.g. by inducing stable secondary structures to the mRNA and affecting the stability of the mRNA. Such sequence changes may alter the expression of an HT susceptibility gene.

A “haplotype,” as described herein, refers to any combination of genetic markers (“alleles”), such as those set forth in tables 3, 4, 5, 7 and 8. A haplotype can comprise two or more alleles.

As it is recognized by those skilled in the art, the same haplotype can be described differently by determining the haplotype defining alleles from different strands e.g. the haplotype rs2221511, rs4940595, rs1522723, rs1395266 (A T C C) described in this invention is the same as haplotype rs2221511, rs4940595, rs1522723, rs1395266 (T A G G) in which the alleles are determined from the other strand or haplotype rs2221511, rs4940595, rs1522723, rs1395266 (T T C C), in which the first allele is determined from the other strand.

The haplotypes described herein, e.g. having markers such as those shown in tables 3, 4, 5, 7 and 8, are found more frequently in individuals with HT than in individuals without HT. Therefore, these haplotypes have predictive value for detecting HT or a susceptibility to HT in an individual. Therefore, detecting haplotypes can be accomplished by methods known in the art for detecting sequences at polymorphic sites.

It is understood that the HT associated at-risk alleles and at-risk haplotypes described in this invention may be associated with other “polymorphic sites” located in HT associated genes of this invention. These other HT associated polymorphic sites may be either equally useful as genetic markers or even more useful as causative variations explaining the observed association of the at-risk alleles and at-risk haplotypes of this invention to HT.

In certain methods described herein, an individual who is at risk of HT is an individual in whom an at-risk allele or an at-risk haplotype is identified. In one embodiment, the at-risk allele or the at-risk haplotype is one that confers a significant risk of HT. In one embodiment, significance associated with an allele or a haplotype is measured by an odds ratio. In a further embodiment, the significance is measured by a percentage. In one embodiment, a significant risk is measured as an odds ratio of at least about 1.2, including but not limited to: 0.1, 0.2, 0.3, 0.4, 0.5, 0.6, 0.7, 0.8, 0.9, 1.2, 1.3, 1.4, 1.5, 1.6, 1.7, 1.8, 1.9, 2.0, 2.5, 3.0, 4.0, 5.0, 10.0, 15.0, 20.0, 25.0, 30.0 and 40.0. In a further embodiment, a significant increase or reduction in risk is at least about 20%, including but not limited to about 25%, 30%, 35%, 40%, 45%, 50%, 55%, 60%, 65%, 70%, 75%, 80%, 85%, 90%, 95% and 98%. In a further embodiment, a significant increase in risk is at least about 50%. It is understood however, that identifying whether a risk is medically significant may also depend on a variety of factors, including the specific disease, the allele or the haplotype, and often, environmental factors.

An at-risk haplotype in, or comprising portions of, the HT risk gene, is one where the haplotype is more frequently present in an individual at risk of HT (affected), compared to the frequency of its presence in a healthy individual (control), and wherein the presence of the haplotype is indicative of HT or susceptibility to HT.

In a preferred embodiment, the method comprises assessing in an individual the presence or frequency of SNPs in, comprising portions of, an HT risk gene, wherein an excess or higher frequency of the SNPs compared to healthy control individuals is indicative that the individual has HT, or is susceptible to HT. See, for example, tables 3, 4, 5, 7 and 8 for SNPs that can form haplotypes that can be used as screening tools. These SNP markers can be identified in at-risk haploptypes. For example, an at-risk haplotype can include microsatellite markers and/or SNPs such as those set forth in tables 3, 4, 5, 7 and 8. The presence of the haplotype is indicative of HT, or a susceptibility to HT, and therefore is indicative of an individual who falls within a target population for the treatment methods described herein.

Consequently, the method of the invention is particularly directed to the detection of one or several of the SNP markers defining the following at-risk haplotypes indicative of HT:

1) rs4845303 (A/T) (SEQ ID NO: 980), rs6428195 (C/G) (SEQ ID NO: 1030) and rs1935659 (A/G) (SEQ ID NO: 637) defining the haplotype ACG;

2) rs1997454 (A/G) (SEQ ID NO: 656), rs2139502 (A/G) (SEQ ID NO: 709) and rs1519991 (A/C) (SEQ ID NO: 542) defining the haplotype AGC;

3) rs1521409 (A/G) (SEQ ID NO: 544), rs10511365 (C/T) (SEQ ID NO: 316) and rs10511366 (C/T) (SEQ ID NO: 317) defining the haplotype ACT;

4) rs7679959 (C/G) (SEQ ID NO: 1178), rs10517338 (C/G) (SEQ ID NO: 381) and rs959297 (A/T) (SEQ ID NO: 1338) defining the haplotype CGA;

5) rs2278677 (A/G) (SEQ ID NO: 749), rs3886091 (C/G) (SEQ ID NO: 899), rs1998167 (A/G) (SEQ ID NO: 657), rs1998168 (A/G) (SEQ ID NO: 658) and rs2235280 (A/G) (SEQ ID NO: 740) defining the haplotype GCAGG;

6) rs10521062 (A/C) (SEQ ID NO: 404), rs10512296 (A/G) (SEQ ID NO: 331), rs1924001 (C/G) (SEQ ID NO: 633) and rs2417359 (A/G) (SEQ ID NO: 784) defining the haplotype AACG;

7) rs10508933 (C/G) (SEQ ID NO: 289), rs10509071 (A/G) (SEQ ID NO: 295) and rs10490967 (A/G) (SEQ ID NO: 94) defining the haplotype GGA;

8) rs10508771 (A/T) (SEQ ID NO: 286), rs3006608 (C/T) (SEQ ID NO: 854), rs10508773 (C/T) (SEQ ID NO: 287) and rs950132 (C/T) (SEQ ID NO: 1325) defining the haplotype TCCC;

9) rs1386486 (C/T) (SEQ ID NO: 472), rs1386485 (A/C) (SEQ ID NO: 471), rs1386483 (A/G) (SEQ ID NO: 470) and rs7977245 (C/T) (SEQ ID NO: 1212) defining the haplotype CAGT;

10) rs276002 (A/G) (SEQ ID NO: 814) and rs274460 (A/G) (SEQ ID NO: 810) defining the haplotype AA;

11) rs1245383 (A/G) (SEQ ID NO: 430), rs2133829 (C/T) (SEQ ID NO: 707), rs2173738 (C/T) (SEQ ID NO: 722), rs2050528 (C/T) (SEQ ID NO: 677) and rs202970 (C/T) (SEQ ID NO: 671) defining the haplotype GCTTC;

12) rs1395266 (C/T) (SEQ ID NO: 476), rs931850 (A/G) (SEQ ID NO: 1303) and rs1522722 (C/T) (SEQ ID NO: 547) defining the haplotype TAC;

13) rs2221511 (A/G) (SEQ ID NO: 733), rs4940595 (G/T) (SEQ ID NO: 986), rs1522723 (C/T) (SEQ ID NO: 548) and rs1395266 (C/T) (SEQ ID NO: 476) defining the haplotype ATCC;

14) rs2825555 (A/G) (SEQ ID NO: 819), rs2825583 (C/T) (SEQ ID NO: 820), rs2825601 (A/G) (SEQ ID NO: 821), rs2825610 (G/T) (SEQ ID NO: 822) and rs1489734 (A/G) (SEQ ID NO: 532) defining the haplotype ATGGA

Monitoring Progress of Treatment

The current invention also pertains to methods of monitoring the effectiveness of a treatment of HT on the expression (e.g. relative or absolute expression) of one or more HT risk genes. The HT susceptibility gene mRNA, the polypeptide it is encoding, or the biological activity of the encoded polypeptide can be measured in a sample of peripheral blood or cells derived therefrom. An assessment of the levels of expression or biological activity of the polypeptide can be made before and during treatment with HT therapeutic agents.

Alternatively the effectiveness of a treatment of HT can be followed by monitoring biological networks and/or metabolic pathways related to one or several polypeptides encoded by HT risk genes listed in table 6. Monitoring biological networks and/or metabolic pathways can be done e.g. by measuring one or several polypeptides from plasma proteome and/or by measuring one or several metabolites from plasma metabolome before and during treatment. Effectiveness of a treatment is evaluated by comparing observed changes in biological networks and or metabolic pathways following treatment with HT therapeutic agents to the data available from healthy subjects.

For example, in one embodiment of the invention, an individual who is a member of the target population can be assessed for response to treatment with an HT inhibitor, by examining the HT risk gene encoding polypeptide biological activity or absolute and/or relative levels of HT risk gene encoding polypeptide or mRNA in peripheral blood in general or specific cell subfractions or combination of cell subfractions.

In addition, variations such as haplotypes or mutations within or near (within one to hundreds of kb) the HT risk gene may be used to identify individuals who are at higher risk for HT to increase the power and efficiency of clinical trials for pharmaceutical agents to prevent or treat HT or its complications. The haplotypes and other variations may be used to exclude or fractionate patients in a clinical trial who are likely to have involvement of another pathway in their HT in order to enrich patients who have pathways involved that are relevant regarding to the treatment tested and boost the power and sensitivity of the clinical trial. Such variations may be used as a pharmacogenetic test to guide selection of pharmaceutical agents for individuals.

Primers, Probes and Nucleic Acid Molecules

“Probes” or “primers” are oligonucleotides that hybridize in a base-specific manner to a complementary strand of nucleic acid molecules. “Base specific manner” means that the two sequences must have a degree of nucleotide complementarity sufficient for the primer or probe to hybridize. Accordingly, the primer or probe sequence is not required to be perfectly complementary to the sequence of the template. Non-complementary bases or modified bases can be interspersed into the primer or probe, provided that base substitutions do not inhibit hybridization. The nucleic acid template may also include “nonspecific priming sequences” or “nonspecific sequences” to which the primer or probe has varying degrees of complementarity. Such probes and primers include polypeptide nucleic acids (Nielsen P E et al, 1991).

A probe or primer comprises a region of nucleic acid that hybridizes to at least about 15, for example about 20-25, and in certain embodiments about 40, 50, or 75 consecutive nucleotides of a nucleic acid of the invention, such as a nucleic acid comprising a contiguous nucleic acid sequence.

In preferred embodiments, a probe or primer comprises 100 or fewer nucleotides, in certain embodiments, from 6 to 50 nucleotides, for example, from 12 to 30 nucleotides. In other embodiments, the probe or primer is at least 70% identical to the contiguous nucleic acid sequence or to the complement of the contiguous nucleotide sequence, for example, at least 80% identical, in certain embodiments at least 90% identical, and in other embodiments at least 95% identical, or even capable of selectively hybridizing to the contiguous nucleic acid sequence or to the complement of the contiguous nucleotide sequence. Often, the probe or primer further comprises a label, e.g. radioisotope, fluorescent compound, enzyme, or enzyme co-factor.

Antisense nucleic acid molecules of the invention can be designed using the nucleotide sequences available e.g. in GenBank database for HT associated genes of table 6 as well as nucleotide sequences containing polymorphic sites listed in tables 2 to 5 and 7 to 11. Antisense oligonucleotides can be constructed using chemical synthesis and enzymatic ligation reactions using procedures known in the art. For example, an antisense nucleic acid molecule (e.g. an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g. phosphorothioate derivatives and acridine substituted nucleotides can be used. Alternatively, the antisense nucleic acid molecule can be produced biologically using an expression vector into which a nucleic acid molecule has been subcloned in an antisense orientation (i.e. RNA transcribed from the inserted nucleic acid molecule will be of an antisense orientation to a target nucleic acid of interest).

The nucleic acid sequences of the HT associated genes of table 6 described in this invention can also be used to compare with endogenous DNA sequences in patients to identify genetic disorders (e.g. a predisposition for, or susceptibility to HT), and as probes, such as to hybridize and discover related DNA sequences or to extract known sequences from a sample. The nucleic acid sequences can further be used to derive primers for genetic fingerprinting, to raise anti-polypeptide antibodies using DNA immunization techniques, and as an antigen to raise anti-DNA antibodies or elicit immune responses. Portions or fragments of the nucleotide sequences identified herein (and the corresponding complete gene sequences) can be used in numerous ways as polynucleotide reagents. For example, these sequences can be used to: (i) map their respective genes on a chromosome; and thus locate gene regions associated with genetic disease; (ii) identify an individual from a minute biological sample (tissue typing); and (iii) aid in forensic identification of a biological sample. Additionally, the nucleotide sequences of the invention can be used to identify and express recombinant polypeptides for analysis, characterization or therapeutic use, or as markers for tissues in which the corresponding polypeptide is expressed, either constitutively, during tissue differentiation, or in diseased states. The nucleic acid sequences can additionally be used as reagents in the screening and/or diagnostic assays described herein, and can also be included as components of kits (e.g. reagent kits) for use in the screening and/or diagnostic assays described herein.

Polyclonal and Monoclonal Antibodies

Polyclonal and/or monoclonal antibodies that specifically bind one form of the gene product but not to the other form of the gene product are also provided. Antibodies are also provided that bind a portion of either the variant or the reference gene product that contains the polymorphic site or sites. The term “antibody” as used herein refers to immunoglobulin molecules and immunologically active portions of immunoglobulin molecules, i.e. molecules that contain an antigen binding site that specifically binds an antigen. A molecule that specifically binds to a polypeptide of the invention is a molecule that binds to that polypeptide or a fragment thereof, but does not substantially bind other molecules in a sample, e.g. a biological sample, which naturally contains the polypeptide. Examples of immunologically active portions of immunoglobulin molecules include F(ab) and F(ab′) fragments which can be generated by treating the antibody with an enzyme such as pepsin. The invention provides polyclonal and monoclonal antibodies that bind to a polypeptide of the invention. The term “monoclonal antibody” or “monoclonal antibody composition” as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

Polyclonal antibodies can be prepared as known by those skilled in the art by immunizing a suitable subject with a desired immunogen, e.g. a polypeptide of the invention or fragment thereof. The antibody titer in the immunized subject can be monitored over time by standard techniques such as with an enzyme linked immunosorbent assay (ELISA) using immobilized polypeptide. If desired, the antibody molecules directed against the polypeptide can be isolated from the mammal (e.g. from blood) and further purified by well-known techniques, such as protein A chromatography to obtain the IgG fraction. At an appropriate time after immunization, e.g. when the antibody titers are highest, antibody-producing cells can be obtained from the subject and used to prepare monoclonal antibodies by standard techniques, such as the hybridoma technique (Kohler G and Milstein C, 1975), the human B cell hybridoma technique (Kozbor D et al, 1982), the EBV-hybridoma technique (Cole S P et al, 1994), or trioma techniques (Hering S et al, 1988). To produce a hybridoma an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cell lines can be applied for the purpose of generating a monoclonal antibody to a polypeptide of the invention (Bierer B et al, 2002). Moreover, the ordinarily skilled worker will appreciate that there are many variations of such methods that would also be useful.

As an alternative to preparing monoclonal antibody-secreting hybridomas, a monoclonal antibody to a polypeptide of the invention can be identified and isolated by screening a recombinant combinatorial immunoglobulin library (e.g. an antibody phage display library) with the polypeptide to thereby isolate immunoglobulin library members that bind the polypeptide (Hayashi N et al, 1995; Hay B N et al, 1992; Huse W D et al, 1989; Griffiths A D et al, 1993). Kits for generating and screening phage display libraries are commercially available.

Additionally, recombinant antibodies, such as chimeric and humanized monoclonal antibodies comprising both human and nonhuman portions, which can be made using standard recombinant DNA techniques, are within the scope of the invention. Such chimeric and humanized monoclonal antibodies can be produced by recombinant DNA techniques known in the art.

In general, antibodies of the invention (e.g. a monoclonal antibody) can be used to isolate a polypeptide of the invention by standard techniques such as affinity chromatography or immunoprecipitation. A polypeptide-specific antibody can facilitate the purification of natural polypeptide from cells and of recombinantly produced polypeptide expressed in host cells. Moreover, an antibody specific for a polypeptide of the invention can be used to detect the polypeptide (e.g. in a cellular lysate, cell supernatant, or tissue sample) in order to evaluate the abundance and pattern of expression of the polypeptide. Antibodies can be used diagnostically to monitor protein levels in tissue such as blood as part of a test predicting the susceptibility to HT or as part of a clinical testing procedure, e.g. to determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, and acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S and ³H.

Diagnostic Assays

The probes, primers and antibodies described herein can be used in methods of diagnosis of HT or diagnosis of a susceptibility to HT, as well as in kits useful for the diagnosis of HT or susceptibility to HT, or to a disease or condition associated with HT.

In one embodiment of the invention, diagnosis of HT or susceptibility to HT (or diagnosis of or susceptibility to a disease or condition associated with HT), is made by detecting one or several of at-risk alleles or at-risk haplotypes or a combination of at-risk alleles and at-risk haplotypes described in this invention in the subject's nucleic acid as described herein.

In one embodiment of the invention, diagnosis of HT or susceptibility to HT (or diagnosis of or susceptibility to a disease or condition associated with HT) is made by detecting one or several polymorphic sites that are associated with at-risk alleles and/or at-risk haplotypes described in this invention, in the subject's nucleic acid. Diagnostically, the most useful polymorphic sites are those altering the polypeptide structure of an HT associated gene due to a frame shift; due to a premature stop codon, due to an aminoacid change or due to abnormal mRNA splicing. Nucleotide changes in a gene resulting in a change in corresponding polypeptide aminoacid sequence in many case alter the physiological properties of a polypeptide by resulting in a polypeptide having altered biological activity/function, distribution or stability. Other diagnostically useful polymorphic sites are those affecting transcription of an HT associated gene or translation of it's mRNA due to altered tissue specifity, altered transcription rate, altered response to physiological status, altered translation efficiency of the mRNA and altered stability of the mRNA. The presence of nucleotide sequence variants altering the polypeptide structure of HT associated genes or altering the expression of HT associated genes is diagnostic for susceptibility to HT.

For diagnostic applications, there may be informative polymorphisms for prediction of disease risk that are in linkage disequilibrium with the functional polymorphism. Such a functional polymorphism may alter splicing sites, affect the stability or transport of mRNA, or otherwise affect the transcription or translation of the nucleic acid. The presence of nucleotide sequence variants associated with functional polymorphism is diagnostic for susceptibility to HT.

While we have genotyped and included a limited number of example SNP markers in the experimental section, any functional, regulatory or other mutation or alteration described above in any of the HT risk genes identified herein is expected to predict the risk of HT.

In diagnostic assays determination of the nucleotides present in one or several of the HT associated SNP markers of this invention, as well as polymorphic sites associated with HT associated SNP markers of this invention, in an individual's nucleic acid can be done by any method or technique which can accurately determine nucleotides present in a polymorphic site. Numerous suitable methods have been described in the art (Kwok P-Y, 2001; Syvanen A-C, 2001). These methods include, but are not limited to, hybridization assays, ligation assays, primer extension assays, enzymatic cleavage assays, chemical cleavage assays and any combinations of these assays. The assays may or may not include PCR, solid phase step, modified oligonucleotides, labeled probes or labeled nucleotides, and the assay may be multiplex or singleplex. As it is obvious in the art the nucleotides present in polymorphic site can be determined from one nucleic acid strand or from both strands.

In another embodiment of the invention, diagnosis of a susceptibility to HT can also be made by examining transcription of one or several HT associated genes. Alterations in transcription can be analyzed by a variety of methods as described in the art, including e.g. hybridization methods, enzymatic cleavage assays, RT-PCR assays and microarrays. A test sample from an individual is collected and the alterations in the transcription of HT associated genes are assessed from the RNA present in the sample. Altered transcription is diagnostic for a susceptibility to HT.

In another embodiment of the invention, diagnosis of a susceptibility to HT can also be made by examining expression and/or structure and/or function of an HT susceptibility polypeptide. A test sample from an individual is assessed for the presence of an alteration in the expression and/or an alteration in structure and/or function of the polypeptide encoded by an HT risk gene, or for the presence of a particular polypeptide variant (e.g. an isoform) encoded by an HT risk gene. An alteration in expression of a polypeptide encoded by an HT risk gene can be for example, an alteration in the quantitative polypeptide expression (i.e. the amount of polypeptide produced); an alteration in the structure and/or function of a polypeptide encoded by an HT risk gene is an alteration in the qualitative polypeptide expression (e.g. expression of a mutant HT susceptibility polypeptide or of a different splicing variant or isoform). In a preferred embodiment, detection of a particular splicing variant encoded by an HT risk gene, or a particular pattern of splicing variants makes diagnosis of the disease or condition associated with HT or a susceptibility to a disease or condition associated with HT possible.

Alterations in expression and/or structure and/or function of an HT susceptibility polypeptide can be determined by various methods known in the art e.g. by assays based on chromatography, spectroscopy, colorimetry, electrophoresis, isoelectric focusing, specific cleavage, immunologic techniques and measurement of biological activity as well as combinations of different assays. An “alteration” in the polypeptide expression or composition, as used herein, refers to an alteration in expression or composition in a test sample, as compared with the expression or composition of polypeptide by an HT risk gene in a control sample. A control sample is a sample that corresponds to the test sample (i.e. is from the same type of cells), and is from an individual who is not affected by HT. An alteration in the expression or composition of the polypeptide in the test sample, as compared with the control sample, is indicative of a susceptibility to HT.

Western blotting analysis using an antibody as described above that specifically binds to a polypeptide encoded by a mutant HT risk gene, or an antibody that specifically binds to a polypeptide encoded by a nonmutant gene, or an antibody that specifically binds to a particular splicing variant encoded by an HT risk gene, can be used to identify the presence in a test sample of a particular splicing variant or isoform, or of a polypeptide encoded by a polymorphic or mutant HT risk gene, or the absence in a test sample of a particular splicing variant or isoform, or of a polypeptide encoded by a nonpolymorphic or nonmutant gene. The presence of a polypeptide encoded by a polymorphic or mutant gene, or the absence of a polypeptide encoded by a nonpolymorphic or nonmutant gene, is diagnostic for susceptibility to HT, as is the presence (or absence) of particular splicing variants encoded by an HT risk gene.

In one embodiment of this method, the level or amount of polypeptide encoded by an HT risk gene in a test sample is compared with the level or amount of the polypeptide encoded by an HT risk gene in a control sample. A level or amount of the polypeptide in the test sample that is higher or lower than the level or amount of the polypeptide in the control sample, such that the difference is statistically significant, is indicative of an alteration in the expression of the polypeptide encoded by an HT risk gene, and is diagnostic for susceptibility to HT. Alternatively, the composition of the polypeptide encoded by an HT risk gene in a test sample is compared with the composition of the polypeptide encoded by an HT risk gene in a control sample (e.g. the presence of different splicing variants). A difference in the composition of the polypeptide in the test sample, as compared with the composition of the polypeptide in the control sample, is diagnostic for susceptibility to HT. In another embodiment, both the level or amount, and the composition of the polypeptide can be assessed in the test sample and in the control sample. A difference in the amount or level of the polypeptide in the test sample compared to the control sample; a difference in composition in the test sample compared to the control sample; or both a difference in the amount or level, and a difference in the composition, is indicative of susceptibility to HT.

In another embodiment, assessment of the splicing variant or isoform(s) of a polypeptide encoded by a polymorphic or mutant HT risk gene can be performed. The assessment can be performed directly (e.g. by examining the polypeptide itself), or indirectly (e.g. by examining the mRNA encoding the polypeptide, e.g. by mRNA profiling). For example, probes or primers as described herein can be used to determine which splicing variants or isoforms are encoded by an HT risk gene mRNA, using standard methods.

The presence in a test sample of a particular splicing variant(s) or isoform(s) associated with HT or risk of HT, or the absence in a test sample of a particular splicing variant(s) or isoform(s) not associated with HT or risk of HT, is diagnostic for a disease or condition associated with an HT risk gene or susceptibility to a disease or condition associated with an HT risk gene. Similarly, the absence in a test sample of a particular splicing variant(s) or isoform(s) associated with HT or risk of HT, or the presence in a test sample of a particular splicing variant(s) or isoform(s) not associated with HT or risk of HT, is diagnostic for the absence of disease or condition associated with an HT risk gene or susceptibility to a disease or condition associated with an HT risk gene.

The invention further pertains to a method for the diagnosis and identification of susceptibility to HT in an individual by identifying an at-risk allele or an at-risk haplotype in an HT risk gene. In one embodiment, the at-risk allele or the at-risk haplotype is an allele or haplotype for which the presence of the haplotype increases the risk of HT significantly. Although it is to be understood that identifying whether a risk is significant may depend on a variety of factors, including the specific disease, the haplotype, and often, environmental factors, the significance may be measured by an odds ratio or a percentage. In a further embodiment, the significance is measured by a percentage. In one embodiment, a significant risk is measured as an odds ratio of 0.8 or less or at least about 1.2, including but not limited to: 0.1, 0.2, 0.3, 0.4, 0.5, 0.6, 0.7, 0.8, 0.9, 1.2, 1.3, 1.4, 1.5, 1.6, 1.7, 1.8, 1.9, 2.0, 2.5, 3.0, 4.0, 5.0, 10.0, 15.0, 20.0, 25.0, 30.0 and 40.0. In a further embodiment an odds ratio of at least 1.2 is significant. In a further embodiment, an odds ratio of at least about 1.5 is significant. In a further embodiment a significant increase or decrease in risk is at least about 1.7. In a further embodiment, a significant increase in risk is at least about 20%, including but not limited to about 25%, 30%, 35%, 40%, 45%, 50%, 55%, 60%, 65%, 70%, 75%, 80%, 85%, 90%, 95% and 98%. In a further embodiment a significant increase or reduction in risk is at least about 50%. It is understood, however, that identifying whether a risk is medically significant may also depend on a variety of factors, including the specific disease, the allele or the haplotype, and often, environmental factors.

The invention also pertains to methods of diagnosing HT or susceptibility to HT in an individual, comprising screening for an at-risk haplotype in the HT risk gene that is more frequently present in an individual susceptible to HT (affected), compared to the frequency of its presence in a healthy individual (control), wherein the presence of the haplotype is indicative of HT or susceptibility to HT. See tables 3, 4, 6, 7 and 8 for SNP markers that comprise haplotypes that can be used as screening tools. SNP markers from these lists represent at-risk haplotypes and can be used to design diagnostic tests for determining susceptibility to HT.

Kits (e.g. reagent kits) useful in the methods of diagnosis comprise components useful in any of the methods described herein, including for example, PCR primers, hybridization probes or primers as described herein (e.g. labeled probes or primers), reagents for genotyping SNP markers, reagents for detection of labeled molecules, restriction enzymes (e.g. for RFLP analysis), allele-specific oligonucleotides, DNA polymerases, RNA polymerases, marker enzymes, antibodies which bind to altered or to nonaltered (native) HT susceptibility polypeptide, means for amplification of nucleic acids comprising one or several HT risk genes, or means for analyzing the nucleic acid sequence of one or several HT risk genes or for analyzing the amino acid sequence of one or several HT susceptibility polypeptides, etc. In one embodiment, a kit for diagnosing susceptibility to HT can comprise primers for nucleic acid amplification of a region in an HT risk gene comprising an at-risk haplotype that is more frequently present in an individual susceptible to HT. The primers can be designed using portions of the nucleic acids flanking SNPs that are indicative of HT.

This invention is based on the principle that one or a small number of genotypings are performed and the sequence variations to be typed are selected on the basis of their ability to predict HT. For this reason any method to genotype sequence variations in a genomic DNA sample can be used.

Thus, the detection method of the invention may further comprise a step of combining information concerning age, gender, the family history of HT, diabetes and hypercholesterolemia, and the medical history concerning CVD or diabetes of the subject with the results obtained from step b) of the method (see claim 1) for confirming the indication obtained from the detection step. Said information may also concern hypercholesterolemia in the family, smoking status, HT in the family, history of CVD, obesity in the family, and waist-to-hip circumference ratio (cm/cm)

The detection method of the invention may also further comprise a step determining blood, serum or plasma cholesterol, HDL cholesterol, LDL cholesterol, triglyceride, apolipoprotein B and AI, fibrinogen, ferritin, transferrin receptor, C-reactive protein, serum or plasma insulin concentration.

The score that predicts the probability of HT may be calculated using a multivariate failure time model or a logistic regression equation. The results from the further steps of the method as described above render possible a step of calculating the probability of developing HT using a logistic regression equation as follows.

Probability of HT=1/[1+e (−(−a+Σ(bi*Xi))], where e is Napier's constant, Xi are variables related to HT, bi are coefficients of these variables in the logistic function, and a is the constant term in the logistic function, and wherein a and bi are preferably determined in the population in which the method is to be used, and Xi are prefereably selected among the variables that have been measured in the population in which the method is to be used. Preferable values for bᵢ are between −20 and 20; and for i between 0 (zero) and 100,000. A negative coefficient bᵢ implies that the marker is risk-reducing and a positive coefficient implies that the marker is risk-increasing.

Xi are binary variables that can have values or are coded as 0 (zero) or 1 (one) such as SNP markers. The model may additionally include any interaction (product) or terms of any variables Xi, e.g. biXi. An algorithm is developed for combining the information to yield a simple prediction of HT as percentage of risk in one year, two years, five years, 10 years or 20 years.

Alternative statistical models are failure-time models such as the Cox's proportional hazards' model, other iterative models and neural networking models.

The test can be applied to test the risk of developing HT in both healthy persons, as a screening or predisposition test, and high-risk persons (who have e.g. family history of HT, central or other type of obesity, lack of physical activity, high sodium intake, high intake of saturated fats, low intake of potassium and/or magnesium, low HDL cholesterol, diabetes mellitus, glucose intolerance, insulin resistance and the metabolic syndrome, elevated inflammatory marker, or any combination of these or an elevated level of any other risk factor for HT).

The method can be used in the prediction and early diagnosis of HT in adult persons, stratification and selection of subjects in clinical trials, and/or stratification and selection of persons for intensified preventive and curative interventions. The aim is to reduce the cost of clinical drug trials and health care.

Pharmaceutical Compositions

The present invention also pertains to pharmaceutical compositions comprising agents described herein, particularly nucleotides in HT risk genes, and/or comprising other splicing variants encoded by HT risk genes; and/or an agent that alters (e.g. enhances or inhibits) HT risk gene expression or HT susceptibility gene polypeptide activity as described herein. For instance, a polypeptide, protein (e.g. a receptor), an agent that alters an HT risk gene expression, or an HT susceptibility polypeptide binding agent or binding partner, fragment, fusion protein or prodrug thereof, or a nucleotide or nucleic acid construct (vector) comprising a nucleotide of the present invention, or an agent that alters HT susceptibility gene polypeptide activity, can be formulated with a physiologically acceptable carrier or excipient to prepare a pharmaceutical composition. The carrier and composition can be sterile. The formulation should suit the mode of administration.

In a preferred embodiment pharmaceutical compositions comprise an agent or agents reversing, at least partially, HT associated changes in biological networks and/or metabolic pathways related to the HT associated genes of this invention (Table 6).

Suitable pharmaceutically acceptable carriers include but are not limited to water, salt solutions (e.g. NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g. lubricants, preservatives, stabilizers, wetting agents, emulsifiers, salts for influencing osmotic pressure, buffers, coloring, flavoring and/or aromatic substances and the like that do not deleteriously react with the active agents.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrolidone, sodium saccharine, cellulose, magnesium carbonate, etc.

Methods of introduction of these compositions include, but are not limited to, intradermal, intramuscular, intraperitoneal, intraocular, intravenous, subcutaneous, topical, oral and intranasal. Other suitable methods of introduction can also include gene therapy (as described below), rechargeable or biodegradable devices, particle acceleration devices (“gene guns”) and slow release polymeric devices. The pharmaceutical compositions of this invention can also be administered as part of a combinatorial therapy with other agents.

The composition can be formulated in accordance with the routine procedures as a pharmaceutical composition adapted for administration to human beings. For example, compositions for intravenous administration are typically solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., which are, if desired, sterilized or mixed with auxiliary agents, e.g. preservatives, stabilizers, wetting agents, buffers or salts for influencing osmotic pressure, etc. The agent may be incorporated into a cosmetic formulation. For topical application, sprayable aerosol preparations wherein the active ingredient, preferably in combination with a solid or liquid inert carrier material, is packaged in a squeeze bottle or in admixture with a pressurized volatile, normally gaseous propellant, e.g. pressurized air, are also suitable.

Agents described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The agents are administered in a therapeutically effective amount. The amount of agents which will be therapeutically effective in the treatment of a particular disorder or condition will depend on the nature of the disorder or condition, and can be determined by standard clinical techniques. In addition, in vitro or in vivo assays may optionally be employed to help identify optimal dosage ranges. The precise dose to be employed in the formulation will also depend on the route of administration, and the severity of the symptoms of HT, and should be decided according to the judgment of a practitioner and each patient's circumstances. Effective doses may be extrapolated from dose-response curves derived from in vitro or animal model test systems.

Methods of Therapy

The present invention encompasses methods of treatment (prophylactic and/or therapeutic) for HT or a susceptibility to HT, such as individuals in the target populations described herein, using an HT therapeutic agent. An “HT therapeutic agent” is an agent that alters (e.g. enhances or inhibits) HT risk affecting polypeptide (enzymatic activity or quantity) and/or an HT risk gene expression, as described herein (e.g. an agonist or antagonist). HT therapeutic agents can alter an HT susceptibility polypeptide activity or nucleic acid expression by a variety of means, for example, by providing additional HT susceptibility polypeptide or by upregulating the transcription or translation of the HT risk gene; by altering posttranslational processing of the HT susceptibility polypeptide; by altering transcription of an HT risk gene splicing variants; or by interfering with an HT susceptibility polypeptide activity (e.g. by binding to an HT susceptibility polypeptide); or by downregulating the transcription or translation of the HT risk gene, or by inhibiting or enhancing the elimination of an HT susceptibility polypeptide.

In particular, the invention relates to methods of treatment for HT or susceptibility to HT (for example, for individuals in an at-risk population such as those described herein); as well as to methods of treatment for manifestations and subtypes of HT.

Representative HT Therapeutic Agents Include the Following:

nucleic acids or fragments or derivatives thereof described herein, particularly nucleotides encoding the polypeptides described herein and vectors comprising such nucleic acids (e.g. a gene, cDNA, and/or mRNA, double-stranded interfering RNA, a nucleic acid encoding an HT susceptibility polypeptide or active fragment or derivative thereof, or an oligonucleotide; for examples see tables 2 through 8;

other polypeptides (e.g. HT susceptibility receptors); HT susceptibility polypeptide binding agents; peptidomimetics; fusion proteins or prodrugs thereof, antibodies (e.g. an antibody to a mutant HT susceptibility polypeptide, or an antibody to a non-mutant HT susceptibility polypeptide, or an antibody to a particular splicing variant encoded by an HT risk gene, as described above); ribozymes; other small molecules;

and other agents that alter (e.g. inhibit or antagonize) an HT risk gene expression or polypeptide activity or that regulate transcription of an HT risk gene splicing variants (e.g. agents that affect which splicing variants are expressed, or that affect the amount of each splicing variant that is expressed);

and other reagents that alter (e.g. induce or agonize) an HT risk gene expression or polypeptide activity or that regulate transcription of an HT risk gene splicing variants (e.g. agents that affect which splicing variants are expressed or that affect the amount of each splicing variant that is expressed).

More than one HT therapeutic agent can be used concurrently, if desired.

The HT therapeutic agent that is a nucleic acid is used in the treatment of HT. The term, “treatment” as used herein, refers not only to ameliorating symptoms associated with the disease, but also preventing or delaying the onset of the disease and also lessening the severity or frequency of symptoms of the disease, preventing or delaying the occurrence of a second episode of the disease or condition; and/or also lessening the severity or frequency of symptoms of the disease or condition. In the case of atherosclerosis, “treatment” also refers to a minimization or reversal of the development of plaques. The therapy is designed to alter (e.g. inhibit or enhance), replace or supplement activity of an HT polypeptide in an individual. For example, an HT therapeutic agent can be administered in order to upregulate or increase the expression or availability of an HT risk gene or of specific splicing variants of an HT susceptibility, gene or, conversely, to downregulate or decrease the expression or availability of an HT risk gene or specific splicing variants of an HT risk gene. Upregulation or increasing expression or availability of a native HT risk gene or of a particular splicing variant could interfere with or compensate for the expression or activity of a defective gene or another splicing variant; downregulation or decreasing expression or availability of a native HT risk gene or of a particular splicing variant could minimize the expression or activity of a defective gene or the particular splicing variant and thereby minimize the impact of the defective gene or the particular splicing variant.

The HT therapeutic agent(s) are administered in a therapeutically effective amount (i.e. an amount that is sufficient to treat the disease, e.g. by ameliorating symptoms associated with the disease, preventing or delaying the onset of the disease, and/or also lessening the severity or frequency of symptoms of the disease). The amount which will be therapeutically effective in the treatment of a particular individual's disorder or condition will depend on the symptoms and severity of the disease and can be determined by standard clinical techniques. In addition, in vitro or in vivo assays may optionally be employed to help identify optimal dosage ranges. The precise dose to be employed in the formulation will also depend on the route of administration and the severity of the disease or disorder, and should be decided according to the judgment of a practitioner and each patient's circumstances. Effective doses may be extrapolated from dose-response curves derived from in vitro or animal model test systems.

In one embodiment, a nucleic acid of the invention (e.g. a nucleic acid encoding an HT susceptibility polypeptide set forth in table 6 optionally comprising at least one polymorphism shown in tables 2 through 11; or another nucleic acid that encodes an HT susceptibility polypeptide or a splicing variant, derivative or fragment thereof, can be used, either alone or in a pharmaceutical composition as described above. For example, an HT risk gene or a cDNA encoding an HT susceptibility polypeptide, either by itself or included within a vector, can be introduced into cells (either in vitro or in vivo) such that the cells produce native HT susceptibility polypeptide. If necessary, cells that have been transformed with the gene or cDNA or a vector comprising the gene or cDNA can be introduced (or re-introduced) into an individual affected with the disease. Thus, cells that in nature lack a native HT risk gene expression and activity, or have mutant HT risk gene expression and activity, or have expression of a disease-associated HT risk gene splicing variant, can be engineered to express an HT susceptibility polypeptide or an active fragment of an HT susceptibility polypeptide (or a different variant of an HT susceptibility polypeptide). In a preferred embodiment, nucleic acid encoding an HT susceptibility polypeptide, or an active fragment or derivative thereof, can be introduced into an expression vector, such as a viral vector, and the vector can be introduced into appropriate cells in an animal. Other gene transfer systems including viral and nonviral transfer systems can be used. Alternatively, nonviral gene transfer methods such as calcium phosphate coprecipitation, mechanical techniques (e.g. microinjection); membrane fusion-mediated transfer via liposomes; or direct DNA uptake, can also be used.

Alternatively, in another embodiment of the invention, a nucleic acid of the invention; a nucleic acid complementary to a nucleic acid of the invention; or a portion of such a nucleic acid (e.g. an oligonucleotide as described below) can be used in “antisense” therapy in which a nucleic acid (e.g. an oligonucleotide) that specifically hybridizes to the mRNA and/or genomic DNA of an HT risk gene is administered or generated in situ. The antisense nucleic acid that specifically hybridizes to the mRNA and/or DNA inhibits expression of the HT susceptibility polypeptide, e.g. by inhibiting translation and/or transcription. Binding of the antisense nucleic acid can be by conventional base pair complementarity, or for example in the case of binding to DNA duplexes, through specific interaction in the major groove of the double helix.

An antisense construct of the present invention can be delivered, for example, as an expression plasmid as described above. When the plasmid is transcribed in the cell it produces RNA that is complementary to a portion of the mRNA and/or DNA which encodes an HT susceptibility polypeptide. Alternatively, the antisense construct can be an oligonucleotide probe that is generated ex vivo and introduced into cells; it then inhibits expression by hybridizing with the mRNA and/or genomic DNA of an HT risk gene. In one embodiment, the oligonucleotide probes are modified oligonucleotides that are resistant to endogenous nucleases, e.g. exonucleases and/or endonucleases, thereby rendering them stable in vivo. Exemplary nucleic acid molecules for use as antisense oligonucleotides are phosphoramidate, phosphothioate and methylphosphonate analogs of DNA. Additionally, general approaches to constructing oligomers useful in antisense therapy are also described by van der Krol A R et al, 1988 and Stein C A and Cohen J S, 1988. With respect to antisense DNA, oligodeoxyribonucleotides derived from the translation initiation site, e.g. between the −10 and +10 regions of an HT risk gene sequence, are preferred.

To perform antisense therapy, oligonucleotides (mRNA, cDNA or DNA) are designed that are complementary to the mRNA encoding an HT susceptibility polypeptide. The antisense oligonucleotides bind to HT susceptibility mRNA transcripts and prevent translation. Absolute complementarity, although preferred, is not required. A sequence “complementary” to a portion of an RNA, as referred to herein, indicates that a sequence has sufficient complementarity to be able to hybridize with the RNA forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid, as described in detail above. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures.

The oligonucleotides used in antisense therapy can be DNA, RNA, or chimeric mixtures or derivatives or modified versions thereof, single-stranded or double-stranded. The oligonucleotides can be modified at the base moiety, sugar moiety, or phosphate backbone, for example, to improve stability of the molecule, hybridization, etc. The oligonucleotides can include other appended groups such as peptides (e.g. for targeting host cell receptors in vivo), or agents facilitating transport across the cell membrane (Letsinger R L et al, 1989; Lemaitre M et al, 1987) or the blood-brain barrier (Jaeger L B and Banks W A, 2004), or hybridization-triggered cleavage agents (van der Krol A R et al, 1988) or intercalating agents. (Zon G, 1988). To this end, the oligonucleotide may be conjugated to another molecule (e.g. a peptide, hybridization triggered cross-linking agent, transport agent, hybridization-triggered cleavage agent).

The antisense molecules are delivered to cells that express an HT risk gene in vivo. A number of methods can be used for delivering antisense DNA or RNA to cells; e.g. antisense molecules can be injected directly into the tissue site, or modified antisense molecules, designed to target the desired cells (e.g. antisense linked to peptides or antibodies that specifically bind receptors or antigens expressed on the target cell surface) can be administered systematically. Alternatively, in a preferred embodiment, a recombinant DNA construct is utilized in which the antisense oligonucleotide is placed under the control of a strong promoter (e.g. pol III or pol II). The use of such a construct to transfect target cells in the patient results in the transcription of sufficient amounts of single stranded RNAs that will form complementary base pairs with the endogenous HT risk gene transcripts and thereby prevent translation of the HT susceptibility mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art and described above. For example, a plasmid, cosmid, YAC or viral vector can be used to prepare the recombinant DNA construct that can be introduced directly into the tissue site. Alternatively, viral vectors can be used which selectively infect the desired tissue, in which case administration may be accomplished by another route (e.g. systemically).

An endogenous HT risk gene expression can be also reduced by inactivating or “knocking out” an HT risk gene or its promoter using targeted homologous recombination (Smithies O et al, 1985; Thomas K R and Capecchi M R, 1987; Thompson S et al, 1989). For example, a mutant, non-functional HT risk gene (or a completely unrelated DNA sequence) flanked by DNA homologous to the endogenous HT risk gene (either the coding regions or regulatory regions of an HT risk gene) can be used, with or without a selectable marker and/or a negative selectable marker, to transfect cells that express an HT risk gene in vivo. Insertion of the DNA construct, via targeted homologous recombination, results in inactivation of the HT risk gene. The recombinant DNA constructs can be directly administered or targeted to the required site in vivo using appropriate vectors, as described above. Alternatively, expression of nonmutant HT risk gene can be increased using a similar method: targeted homologous recombination can be used to insert a DNA construct comprising a nonmutant, functional HT risk gene (e.g. any gene shown in table 6 that may optionally comprise at least one polymorphism shown in tables 2 through 11), or a portion thereof, in place of a mutant HT risk gene in the cell as described above. In another embodiment, targeted homologous recombination can be used to insert a DNA construct comprising a nucleic acid that encodes an HT susceptibility polypeptide variant that differs from that present in the cell.

Alternatively, an endogenous HT risk gene expression can be reduced by targeting deoxyribonucleotide sequences complementary to the regulatory region of an HT risk gene (i.e. the HT risk gene promoter and/or enhancers) to form triple helical structures that prevent transcription of an HT risk gene in target cells in the body (Helene C, 1991; Helene C et al, 1992; Maher L J, 1992). Likewise, the antisense constructs described herein can be used in the manipulation of tissue, by antagonizing the normal biological activity of one of the HT proteins, e.g. tissue differentiation both in vivo and for ex vivo tissue cultures. Furthermore, the anti-sense techniques (e.g. microinjection of antisense molecules, or transfection with plasmids whose transcripts are anti-sense with regard to an HT mRNA or gene sequence) can be used to investigate the role of an HT risk gene in developmental events, as well as the normal cellular function of an HT risk gene in adult tissue. Such techniques can be utilized in cell culture, but can also be used in the creation of transgenic animals.

In yet another embodiment of the invention, other HT therapeutic agents as described herein can also be used in the treatment or prevention of HT. The therapeutic agents can be delivered in a composition, as described above, or by themshelves. They can be administered systemically, or can be targeted to a particular tissue. The therapeutic agents can be produced by a variety of means including chemical synthesis; recombinant production; in vivo production, e.g. a transgenic animal (Meade H et al, 1990) and can be isolated using standard means such as those described herein.

A combination of any of the above methods of treatment (e.g. administration of non-mutant HT susceptibility polypeptide in conjunction with antisense therapy targeting mutant HT susceptibility mRNA; administration of a first splicing variant encoded by an HT risk gene in conjunction with antisense therapy targeting a second splicing encoded by an HT risk gene), can also be used.

The invention will be further described by the following nonlimiting examples. The teachings of all publications cited herein are incorporated herein by reference in their entirety.

### EXPERIMENTAL SECTION

East Finnish HT Patients and Phenotype Characterization

The subjects were participants of the Kuopio Ischaemic Heart Disease Risk Factor Study (KIHD), which is an ongoing prospective population-based study designed to investigate risk factors for chronic diseases, including HT and CVD, in middle-aged men (Salonen J T 1988, Salonen J T et al 1998, 1999, Tuomainen T-P et al 1999). The study population was a random age-stratified sample of men living in Eastern Finland who were 42, 48, 54 or 60 years old at baseline examinations in 1984-1989. A total of 2682 men were examined during 1984-89. The male cohort was complemented by a random population sample of 920 women first examined during 1998-2001, at the time of the 11-year follow up of the male cohort. The recruitment and examination of the subjects has been described previously in detail (Salonen J T, 1988). The University of Kuopio Research Ethics Committee approved the study. All participants gave their written informed consent.

The analyses are based on logistic modeling in a case-control set of 81 cases with HT (SBP 140 mmHg or more or DBP 90 mmHg or more or antihypertensive medication) and HT in either sibling or parent, and 82 controls who had neither HT nor family history of HT, both from the KIHD cohort. Three of the subjects (two cases, one control) were women, 160 were men. Thirty-eight of the 81 cases had antihypertensive medication at the time of BP measurements in the KIHD baseline examination.

HT was defined as either systolic BP (SBP)≧140 mmHg or diastolic BP (DBP)≧90 mmHg or antihypertensive medication. Both BPs were measured in the morning by a nurse with a random-zero mercury sphygmomanometer. The measuring protocol included three measurements in supine, one in standing and two in sitting position with 5-minutes intervals. The mean of all six measurements were used as SBP and DBP (Salonen J T et al, 1998). The family history of HT was defined positive, if either father, the mother or a sibling of the study subject had reported a history or prevalent hypertension.

TABLE 1
Selected characteristics of the cases and controls

| | Hypertensive cases (n = 81) | | | Normotensive controls (n = 82) | | |
|---|---|---|---|---|---|---|
| | Mean | Min | Max | Mean | Min | Max |
| Age (years) | 54.6 | 42.1 | 71.9 | 54.6 | 42.2 | 61.1 |
| Cigarettes/day | 5.3 | 0 | 40 | 7.4 | 0 | 40 |
| S-Cholesterol (mmol/L) | 6.2 | 3.8 | 9.1 | 6.0 | 3.2 | 8.7 |
| S-HDL-Chol (mmol/L) | 1.21 | 0.82 | 2.15 | 1.34 | 0.76 | 2.77 |
| B-Glucose (mmol/L) | 5.13 | 3.3 | 12.6 | 4.55 | 3.5 | 5.9 |
| S-Insulin (U/L) | 14.7 | 4.7 | 59.6 | 9.33 | 1.7 | 22.5 |
| Mean SBP (mmHg) | 140.0 | 110.0 | 182.33 | 124.5 | 99.0 | 148.33 |
| Mean DBP (mmHg) | 92.1 | 63.3 | 122.3 | 81.3 | 66.0 | 94.3 |

In table 1 selected characteristics of the cases and controls are summarized. Age and tobacco smoking were recorded on a self-administered questionnaire checked by an interviewer. Fasting blood glucose was measured using a glucose dehydrogenase method after precipitation of proteins by trichloroacetic acid. Serum insulin was determined with a Novo Biolabs radioimmunoassay kit (Novo Nordisk). HDL fractions were separated from fresh serum by combined ultracentrifugation and precipitation. The cholesterol contents of lipoprotein fractions and serum triglycerides were measured enzymatically. Fibrinogen was measured based on the clotting of diluted plasma with excess thrombin.

Adulthood socioeconomical status (SES) is an index comprised of measures of education, occupation, income and material living conditions. The scale is inverse, low score corresponding to high SES. These data have been collected by a self administered questionnaire.

Serum ferritin was assessed with a commercial double antibody radioimmunoassay (Amersham International, Amersham, UK). Lipoproteins, including high density lipoprotein (HDL) and low density lipoprotein (LDL), were separated from fresh serum samples by ultracentrifugation followed by direct very low density lipoprotein (VLDL) removal and LDL precipitation (Salonen et al 1991). Cholesterol concentration was then determined enzymically. Serum C-reactive protein was measured by a commercial high-sensitive immunometric assay (Immulite High Sensitivity CR Assay, DPC, Los Angeles).

Genomic DNA Isolation and Quality Testing

High molecular weight genomic DNA samples were extracted from frozen venous whole blood using standard methods, and dissolved in standard TE buffer. The quantity and purity of each DNA sample was evaluated by measuring the absorbance at 260 and 280 nm and integrity of isolated DNA samples was evaluated with 0.9% agarose gel electrophoresis and Ethidiumbromide staining. A sample was qualified for genome wide scan (GWS) analysis if the A260/A280 ratio was >1.7 and the average size of isolated DNA was over 20 kb in agarose gel electrophoresis. Before GWS, analysis samples were diluted to a concentration of 50 ng/μl in reduced EDTA TE buffer (TEKnova).

Genome-Wide Scan

Genotyping of SNP markers was performed using the technology access version of Affymetrix GeneChip® human mapping 100 k system. The assay consisted of two arrays, Xba and Hind, which were used to genotype over 126,000 SNP markers from each DNA sample. The assays were performed according to the instructions provided by the manufacturer. A total of 250 ng of genomic DNA was used for each individual assay. The DNA sample was digested with either Xba I or Hind III enzyme (New England Biolabs, NEB) in the mixture of NE Buffer 2 (1 x; NEB), bovine serum albumin (1 x; NEB), and either Xba I or Hind III (0,5 U/μl; NEB) for 2h at +37° C. followed by enzyme inactivation for 20 min at +70° C. Xba I or Hind III adapters were then ligated to the digested DNA samples by adding Xba or Hind III adapter (0,25 μM, Affymetrix), T4 DNA ligase buffer (1 x; NEB), and T4 DNA ligase (250 U; NEB). Ligation reactions were allowed to proceed for 2h at +16° C. followed by 20 min incubation at +70° C. Each ligated DNA sample was diluted with 75 μl of molecular biology-grade water (BioWhittaker Molecular Applications/Cambrex).

Diluted ligated DNA samples were subjected to four identical 100 μl volume polymerase chain reactions (PCR) by implementing a 10 μl aliquot of DNA sample with Pfx Amplification Buffer (1 x; Invitrogen), PCR Enhancer (1 x; Invitrogen), MgSO₄ (1 mM; Invitrogen), dNTP (300 μM each; Takara), PCR primer (1 μM; Affymetrix), and Pfx Polymerase (0,05 U/μl; Invitrogen). The PCR was allowed to proceed for 3 min at +94° C., followed by 30 cycles of 15 sec at +94° C., 30 sec at +60° C., 60 sec at +68° C., and finally for the final extension for 7 min at +68° C. The performance of the PCR was checked by standard 2% agarose gel electrophoresis in 1×TBE buffer for 1 h at 120V.

PCR products were purified according to the Affymetrix manual using MinElute 96 UF PCR Purification kit (Qiagen) by combining all four PCR products of an individual sample into the same purification reaction. The purified PCR products were eluted with 40 μl of EB buffer (Qiagen), and the yields of the products were measured at the absorbance 260 nm. A total of 40 μg of each PCR product was then subjected to fragmentation reaction consisting of 0.2 U/μl fragmentation reagent (Affymetrix) in 1×Fragmentation Buffer. The fragmentation reaction was allowed to proceed for 35 min at +37° C. followed by 15 min incubation at +95° C. for enzyme inactivation. Completeness of fragmentation was checked by running an aliquot of each fragmented PCR product in 4% agarose 1×TBE (BMA Reliant precast) for 30-45 min at 120V.

Fragmented PCR products were then labeled using 1×Terminal Deoxinucleotidyl Transferase (TdT) buffer (Affymetrix), GeneChip DNA Labeling Reagent (0.214 mM; Affymetrix), and TdT (1,5 U/μl; Affymetrix) for 2 h at +37° C. followed by 15 min at +95° C. Labeled DNA samples were combined with hybridization buffer consisting of 0.056 M MES solution (Sigma), 5% DMSO (Sigma), 2.5×Denhardt's solution (Sigma), 5.77 mM EDTA (Ambion), 0.115 mg/ml Herring Sperm DNA (Promega), 1×Oligonucleotide Control reagent (Affymetrix), 11.5 μg/ml Human Cot-1 (Invitrogen), 0.0115% Tween-20 (Pierce), and 2.69 M Tetramethyl Ammonium Chloride (Sigma). DNA-hybridization buffer mix was denatured for 10 min at +95° C., cooled on ice for 10 sec and incubated for 2 min at +48° C. prior to hybridization onto corresponding Xba or Hind GeneChip® array. Hybridization was completed at +48° C. for 16-18 h at 60 rpm in an Affymetrix GeneChip Hybridization Oven. Following hybridization, the arrays were stained and washed in GeneChip Fluidics Station 450 according to fluidics station protocol Mapping10Kv1_{—}450 as recommended by the manufacturer. Arrays were scanned with GeneChip 3000 Scanner and the genotype calls for each of the SNP markers on the array were generated using Affymetrix Genotyping Tools (GTT) software. The confidence score in SNP calling algorithm was adjusted to 0.20.

Initial SNP Selection for Statistical Analysis

Prior to the statistical analysis, SNP quality was assessed on the basis of three values: the call rate (CR), minor allele frequency (MAF), and Hardy-Weinberg equilibrium (H-W). The CR is the proportion of samples genotyped successfully. It does not take into account whether the genotypes are correct or not. The call rate was calculated as: CR=number of samples with successful genotype call/total number of samples. The MAF is the frequency of the allele that is less frequent in the study sample. MAF was calculated as: MAF=min(p, q), where p is frequency of the SNP allele ‘A’ and q is frequency of the SNP allele ‘B’; p=(number of samples with “AA”-genotype+0.5*number of samples with “AB”-genotype)/total number of samples with successful genotype call; q=1-p. SNPs that are homozygous (MAF=0) cannot be used in genetic analysis and were thus discarded. H-W equilibrium is tested for controls. The test is based on the standard Chi-square test of goodness of fit. The observed genotype distribution is compared with the expected genotype distribution under H-W equilibrium. For two alleles this distribution is p², 2 pq, and q² for genotypes ‘AA’, ‘AB’ and ‘BB’, respectively. If the SNP is not in H-W equilibrium it can be due to genotyping error or some unknown population dynamics (e.g. random drift, selection).

Only the SNPs that had CR>50%, MAF>1%, and were in H-W equilibrium (Chi-square test statistic<23.93) were used in the statistical analysis. A total of 107,895 SNPs fulfilled the above criteria and were included in the statistical analysis.

Statistical Methods

Single SNP Analysis

Differences in allele distributions between cases and controls were screened for all 107,895 SNPs. The screening was carried out using the standard Chi-square independence test with 1 df (allele distribution, 2×2 table). SNPs that gave a P-value less than 0.005 (Chi-square distribution with 1 df of 7.88 or more) were considered as statistically significant and selected for further analysis. There were 529 SNPs that fulfilled this criterium.

Haplotype Analysis

The data set was analyzed with a haplotype pattern mining algorithm either with HPM-G software (Sevon P et al, 2004) or with HPM software (Toivonen HT et al, 2000). For HPM software, genotypes must be phase known to determine which alleles come from the mother and which from the father. Without family data, phases must be estimated based on population data. We used HaploRec-program (Eronen L et al, 2004) to estimate the phases. HPM-G and HPM are very fast and can handle a large number of SNPs in a single run

The difference between HPM and HPM-G is that HPM-G can use phase unknown genotypic data and HPM uses phase known (or estimated by HaploRec or similar program) data. HPM-G finds all haplotype patterns that fit the genotype configuration. For phase-known data HPM finds all haplotype patterns that are in concordance with the phase configuration. The length of the haplotype patterns can vary. As an example, if there are four SNPs and an individual has alleles A T for SNP1, C C for SNP2, C G for SNP3, and A C for SNP4, then HPM-G considers haplotype patterns (of length 4 SNPs): ACCA, TCGC, TCCA, ACGC, ACGA, TCCC, TCGA, ACCC. HPM considers only haplotype patterns that are in concordance with the estimated phase (done by HaploRec). If the estimated phase is ACGA (from the mother/father) and TCCC (from the father/mother) then HPM considers only two patterns (of length 4 SNPs): ACGA and TCCC.

A SNP is scored based on the number of times it is included in a haplotype pattern that differs between cases and controls (a threshold Chi-square value can be selected by the user). Significance of the score values is tested based on permutation tests.

Several parameters can be modified in the HPM-G and HPM programs including the Chi-square threshold value (−x), the maximum haplotype pattern length (−1), the maximum number of wildcards that can be included in a haplotype pattern (−w), and the number of permutation tests in order to estimate the P-value (−p). Wildcards allow gaps in haplotypes. The HPM-G program was run with the following parameter settings: haplotype analysis with 5 SNPs (−x9−15 −w1 −p10000). HaploRec+HPM was run with the following parameter settings: haplotype analysis with 5 SNPs (−x9−15 −w1 −p10000). HPM-G analysis was based on the order of the SNP given in dbSNP122 and HaploRec+HPM was based on the order of the SNP given in dbSNP123. Based on 10,000 replicates (−p10000) in the HPM-G analyses 570 SNPs were significant at P-value less than 0.005 and 642 SNPs were significant in the HPM analysis.

Definition of Terms Used in the Haplotype Analysis Results

The term “haplotype genomic region” or “haplotype region” refers to a genomic region that has been found significant in the haplotype analysis (HPM, HPMG or similar statistical method/program). The haplotype region is defined as 100 Kbp up/downstream from the physical position of the first/last SNP that was included in the statistical analysis (haplotype analysis) and was found statistically significant. This region is given in base pairs based on the given genome build e.g. SNP physical position (base pair position) according to NCBI Human Genome Build 35.

The term “haplotype” as described herein, refers to any combination of alleles e.g. A T C C that is found in the given genetic markers e.g rs2221511, rs4940595, rs1522723, rs1395266. A defined haplotype gives the name of the genetic markers (dbSNP rs-id for the SNPs) and the alleles. As it is recognized by those skilled in the art, the same haplotype can be described differently by determining alleles from different strands e.g. the haplotype rs2221511, rs4940595, rs1522723, rs1395266 (A T C C) is the same as haplotype rs2221511, rs4940595, rs1522723, rs1395266 (T A G G) in which the alleles are determined from the other strand, or haplotype rs2221511, rs4940595, rs1522723, rs1395266 (T T C C), in which the first allele is determined from the other strand.

The haplotypes described herein, e.g. having markers such as those shown in tables 3, 4, 5, 7 and 8, are found more frequently in individuals with HT than in individuals without HT. Therefore, these haplotypes have predictive value for detecting HT or a susceptibility to HT in an individual. Therefore, detecting haplotypes can be accomplished by methods known in the art for detecting sequences at polymorphic sites.

It is understood that the HT associated at-risk alleles and at-risk haplotypes described in this invention may be associated with other “polymorphic sites” located in HT associated genes of this invention. These other HT associated polymorphic sites may be either equally useful as genetic markers or even more useful as causative variations explaining the observed association of at-risk alleles and at-risk haplotypes of this invention to HT.

Multivariate Modeling

For modeling for hypertension as a binary outcome, the 734 strongest predicting SNP markers from the individual SNP analysis and 14 strongest haplotypes from the HPM analysis were tested for entry to the model. These were recoded as 0, if homozygote of the major allele, 1, if heterozygote and 2, if homozygote of the minor allele. A multivariate binary logistic function regression analysis was used to: a) Find the SNPs that were most predictive of HT and b) Construct a multivariate model that predicted HT the strongest. A forward step-up model construction was used with p-value to enter of 0.01 and p-value to exclude from the model of 0.02. The predictivity of the models was estimated by two methods: the Nagelkerke R square and the reclassification of the subjects to cases and controls on the basis of the logistic model contructed. The predicted probability used as cut-off was 0.5. A data reduction analysis was carried out by step-down and step-up logistic modeling.

Multivariate least-squares linear regression modeling was used to identify the SNP markers that were most strongly associated with the mean systolic and diastolic blood pressure as quantitative traits. A forward step-up model construction was used with p-value to enter of 0.001 and p-value to exclude from the model of 0.005.

The statistical software used was SPSS for Windows, version 11.5.

Results

In table 2 (on CD) are summarized the characteristics of the SNP markers with the strongest association with HT in the individual marker analysis (n=529). SNP identification numbers are according to NCBI dbSNP database build 124. Physical positions of SNP markers are according to NCBI Human Genome Build 35. Gene locus as reported by NCBI dbSNP database build 124. SNP flanking sequence provided by Affymetrix “csv” commercial access Human Mapping 100K array annotation files.

In table 3 (on CD) are summarized the characteristics of the haplotype genomic regions with the strongest association with HT in the HPM-G analysis with 5 SNPs. SNP identification numbers are according to NCBI dbSNP database build 124. Physical positions of SNP markers are according to NCBI Human Genome Build 35. Associated genes are those genes positioned within 100 Kbp up/downstream from the physical position of the SNPs bordering the haplotype genomic region found using NCBI MapViewer, based on NCBI Human Genome Build 35. SNP flanking sequence provided by Affymetrix “csv” commercial access Human Mapping 100K array annotation files.

In table 4 (on CD) are summarized the characteristics of the haplotype genomic regions with the strongest association with HT in the HaploRec+HPM analysis with 5 SNPs. SNP identification numbers are according to NCBI dbSNP database build 124. Physical positions of SNP markers are according to NCBI Human Genome Build 35. Associated genes are those genes positioned within 100 Kbp up/downstream from the physical position of the SNPs bordering the haplotype genomic region found using NCBI MapViewer, based on NCBI Human Genome Build 35. SNP flanking sequence provided by Affymetrix “csv” commercial access Human Mapping 100K array annotation files.

In table 5 (on CD) are listed haplotype blocks with the strongest association with HT based on HaploRec+HPM analysis (n=14). SNP identification numbers are according to NCBI dbSNP database build 124.

In table 6 are listed all genes found associated with HT according to point wise and haplotype analyses (n=722). Names of genes are according to HUGO Gene Nomenclature Committee (HGNC).

In table 7 are listed the SNP-markers and haplotypes that best predicted risk of familial HT in a multivariate logistic model. SNP identification numbers are according to NCBI dbSNP database build 124. The 8-variable model predicts 91.4% of familial HT correctly. The statistics are based on 81 KIHD participants who were hypertensive in the KIHD baseline examination (SBP 140 mmHg or more or DBP 90 mmHg or more or antihypertensive medication) and either sibling or parent had HT and 82 KIHD participants who neither had HT at KIHD baseline nor had family history of HT. The controls were matched according to age.

In table 8 are listed the SNP-markers, haplotypes and phenotypic data that best predicted risk of familial HT in a multivariate logistic model. SNP identification numbers are according to NCBI dbSNP database build 124. The 12-variable model, including two haplotypes, five SNP markers and two phenotypic variables, predicted 87.1% of familial HT correctly. The strongest loci pinpointed by the multivariate logistic models were SERPINs B3, B4, B7 and B11 and EPC1, OR1J4 and LOC401406, 439953, 441550 and 441551.

Table 9 presents a multivariate linear regression model of the strongest SNPs predicting the mean systolic and diastolic BP. Tables 10 and 11 show the means and standard deviations of the mean systolic (Table 10) and diastolic (Table 11) BP in the genotypes of the strongest SNP markers, which predicted BP the strongest in both the univariate single-SNP, haplotype and multivariate analyses. The rank order of markers is according to the strength of association with the diastolic BP. The strongest pinpointed genes concerning BP as quantitative trait were SERPINS B3, B7 and B11, A100A7, S100A6, FARS1, SPOCK3, and TLL1.

Implications and Conclusions

We have found 1365 SNP markers associated with the risk of HT and/or blood pressure in a population-based set of familial cases and healthy controls without family history. Of these, 529 were identified in the analysis of individual SNPs and 1080 in haplotype pattern mining or haplotype analysis. Of the 1365 markers, 244 predicted HT in both types of statistical analysis. We further identified SNP markers, which predict in a multivariate logistic model virtually fully the development of HT.

The results of the point wise and haplotype analyses identified a total of 722 genes associated with HT, of which 330 genes had at least one of the 1365 SNP markers physically linked to the gene.

Thus, we have discovered a total of 722 HT genes, in which any genetic markers can be used to predict HT, and thus these markers can be used as part of molecular diagnostic tests of HT predisposition. In addition, we have disclosed a set of 1365 SNP markers which are predictive of HT. The markers can also be used as part of pharmacogenetic tests predicting the efficacy and adverse reactions of antihypertensive agents and compounds. The genes discovered are also targets to new therapies of HT, such as drugs. Other therapies are molecular, including gene transfer. The new genes can also be used to develop and produce new transgenic animals for studies of antihypertensive agents and compounds.

While this invention has been particularly shown and described with reference to preferred embodiments thereof, it will be understood by those skilled in the art that various changes in form and details may be made therein without departing from the spirit and scope of the invention as defined by the appended claims.

### REFERENCES

American Heart Association. Heart Disease and Stroke Statistics—2004 Update. http://www.americanheart.org/downloadable/heart/1079736729696HDSStats2004UpdateRE V3-19-04.pdf (Accessed 14.07.04)

Beltran A, McVeigh G, Morgan D, Glasser S P, Neutel J M, Weber M, Finkelstein S M, Cohn J N. 2001. Arterial compliance abnormalities in isolated systolic hypertension. Am J Hypertens. 14:1007-11.

Berry C, Brosnan M J, Fennell J, Hamilton C A, Dominiczak A F. 2001. Oxidative stress and vascular damage in hypertension. Curr Opin Nephrol Hypertens. 10:247-55.

Bierer B, Coligan J E, Margulies D H, Shevach E M, Strober W. 2002. Current Protocols in Immunology. NY:John Wiley & Sons.

Blacher J, Asmar R, Djane S, London G M, Safar M E. 1999. Aortic pulse wave velocity as a marker of cardiovascular risk in hypertensive patients. Hypertension. 33:1111-7.

Brasier A R, Recinos A, Eledrisi M S. 2002. Vascular inflammation and the renin-angiotensin system. Arterioscler Thromb Vasc Biol 22:1257-1266.

Chobanian A V, Bakris G L, Black H R, Cushman W C, Green L A, Izzo J L Jr, Jones D W, Materson B J, Oparil S, Wright J T Jr, Roccella E J; National Heart, Lung, and Blood Institute Joint National Committee on Prevention, Detection, Evaluation, and Treatment of High Blood Pressure; National High Blood Pressure Education Program Coordinating Committee. 2003. The Seventh Report of the Joint National Committee on Prevention, Detection, Evaluation, and Treatment of High Blood Pressure: the JNC 7 report. JAMA. 289:2560-72. Chobanian A V, Hill M. 2000. National Heart, Lung, and Blood Institute workshop on sodium and blood pressure. Hypertension 35:858-863.

Cole S P, Campling B G, Atlaw T, Kozbor D, Roder J C. 1984. Human monoclonal antibodies. Mol Cell Biochem. 62:109-20.

Cowley A W Jr, Roman R J. 1996. The role of the kidney in hypertension. JAMA 275:1581-1589.

Cowley A W Jr. 1992. Long-term control of arterial blood pressure. Physiol Rev. 72:231-300.

Cusi D, Barlassina C, Azzani T, Casari G, Citterio L, Devoto M, Glorioso N, Lanzani C, Manunta P, Righetti M, Rivera R, Stella P, Troffa C, Zagato L, Bianchi G. 1997.

Polymorphisms of alpha-adducin and salt sensitivity in patients with essential hypertension. Lancet. 349:1353-7.

Dodt C, Breckling U, Derad I, Fehm H L, Born J. 1997. Plasma epinephrine and norepinephrine concentrations of healthy humans associated with nighttime sleep and morning arousal. Hypertension. 30:71-6.

Esler M, Rumantir M, Lambert G, Kaye D. 2001. The sympathetic neurobiology of essential hypertension: disparate influences of obesity, stress, and noradrenaline transporter dysfunction? Am J Hypertens 14:139S-146S.

European Society of Hypertension-European Society of Cardiology Guidelines Committee. 2003. 2003 European Society of Hypertension-European Society of Cardiology guidelines for the management of arterial hypertension. J Hypertens. 21:1011-53.

Feldman R D, Gros R. 1998. Impaired vasodilator function in hypertension. The role of alterations in receptor-G protein coupling. Trends Cardiovasc Med 8:297-305.

Folkow B, Hallack M, Lundgren Y, Weiss L. 1970. Background of increased flow resistance and vascular reactivity in spontaneously hypertensive rats. Acta Physiol Scand 80:93-106.

Fuentes R, Ilmaniemi N, Laurikainen E, Tuomilehto J, Nissinen A. 2000. Hypertension in developing economies: a review of population-based studies carried out from 1980 to 1998. J Hypertens.18:521-9.

Fuentes Rivera, Ricardo Marcelo. 2003. Familial aggregation and tracking of blood pressure, body mass index and serum total cholesterol during childhood. A prospective family study in eastern Finland. Doctoral Dissertation. Kuopio University Publications D. Medical Sciences 296: 139 p. ISBN 951-781-896-3; ISSN 1235-0303.

Griffiths A D, Malmqvist M, Marks J D, Bye J M, Embleton M J, McCafferty J, Baier M, Holliger K P, Gorick B D, Hughes-Jones N C, et al. 1993. Human anti-self antibodies with high specificity from phage display libraries. EMBO J. 12:725-34.

Guyton A C. 1961. Physiologic regulation of arterial pressure. Am J Cardiol 8:401-407.

Guyton A C. 1991. Blood pressure control-special role of the kidneys and body fluids. Science 252:1813-6.

Guyton A C. 1992. Kidneys and fluids in pressure regulation. Hypertension 19(Suppl 1):I2-18.

Hall J E, Brands M W, Henegar J R. 1999. Angiotensin II and long-term arterial pressure regulation. J Am Soc Nephrol 10:S258-S265.

Harrap S B, Cumming A D, Davies D L, Foy C J, Fraser R, Kamitani A, Connor J M, Lever A F, Watt G C. 2000. Glomerular hyperfiltration, high renin, and low- extracellular volume in high blood pressure. Hypertension. 35:952-7.

Hay B N, Sorge J A, Shopes B. 1992. Bacteriophage cloning and Escherichia coli expression of a human IgM Fab. Hum Antibodies Hybridomas. 3:81-5.

Hayashi N, Kipriyanov S, Fuchs P, Welschof M, Dorsam H, Little M. 1995. A single expression system for the display, purification and conjugation of single-chain antibodies. Gene. 160:129-30.

Helene C, Thuong N T, Harel-Bellan A. 1992. Control of gene expression by triple helix-forming oligonucleotides. The antigene strategy. Ann N Y Acad Sci. 660:27-36.

Helene C. 1991. The anti-gene strategy: control of gene expression by triplex-forming-oligonucleotides. Anticancer Drug Des. 6:569-84.

Hering S, Karawajew L, Pasternak G. 1988. Raji-K562 hybrids and their use for trioma production. Biomed Biochim Acta. 47:211-6.

Hopkins P N, Hunt S C. 2003. Genetics of hypertension. Genet Med. 5:413-29.

Hunt S C, Cook N R, Oberman A, Cutler J A, Hennekens C H, Allender P S, Walker W G, Whelton P K, Williams R R. 1998. Angiotensinogen genotype, sodium reduction, weight loss, and prevention of hypertension: trials of hypertension prevention, phase II. Hypertension. 32:393-401.

Hunt S C, Geleijnse J M, Wu L L, Witteman J C, Williams R R, Grobbee D E. 1999. Enhanced blood pressure response to mild sodium reduction in subjects with the 235T variant of the angiotensinogen gene. Am J Hypertens. 12:460-6.

Huse W D, Sastry L, Iverson S A, Kang A S, Alting-Mees M, Burton D R, Benkovic S J, Lemer R A. 1989. Generation of a large combinatorial library of the immunoglobulin repertoire in phage lambda. Science. 246:1275-81.

Intengan H D, Schiffrin E L. 2001. Vascular remodeling in hypertension. Role of apoptosis, inflammation and fibrosis. Hypertension 38:581-587.

Jaeger L B, Banks W A. 2004. Antisense therapeutics and the treatment of CNS disease. Front Biosci. 9:1720-7.

Johns D G, Dorrance A M, Leite R, Weber D S, Webb R C. 2000. Novel signaling pathways contributing to vascular changes in hypertension. J Biomed Sci. 7:431-43.

Julius S, Krause L, Schork N J, Mejia A D, Jones K A, van de Ven C, Johnson E H, Sekkarie M A, Kjeldsen S E, Petrin J, et al. 1991. Hyperkinetic borderline hypertension in Tecumseh, Michigan. J Hypertens. 9:77-84.

Kohler G, Milstein C. 1975. Continuous cultures of fused cells secreting antibody of predefined specificity. Nature 256:495-497.

Koivukoski L, Fisher S A, Kanninen T, Lewis C M, von Wowem F, Hunt S, Kardia S L, Levy D, Perola M, Rankinen T, Rao D C, Rice T, Thiel B A, Melander O. 2004. Meta-analysis of genome-wide scans for hypertension and blood pressure in Caucasians shows evidence of susceptibility regions on chromosomes 2 and 3. Hum Mol Genet. 13:2325-32.

Koller A. 2002. Signaling pathways of mechanotransduction in arterioloar endothelium and smooth muscle cells in hypertension. Microcirculation 9:277-294.

Kozbor D, Lagarde A E, Roder J C. 1982. Human hybridomas constructed with antigen-specific Epstein-Barr virus-transformed cell lines. Proc Natl Acad Sci U S A. 79:6651-5.

Kranzhofer R, Schmidt J, Pfeiffer C A, Hagl S, Libby P, Kubler W. 1999. Angiotensin induces inflammatory activation of human vascular smooth muscle cells. Arterioscler Thromb Vasc Biol. 19:1623-9.

Kwok P-Y. 2001. Methods for genotyping single nucleotide polymorphisms. Ann Rev Genomics Hum Genet. 2:235-258.

Lemaitre M, Bayard B, Lebleu B. 1987. Specific antiviral activity of a poly(L-lysine)-conjugated oligodeoxyribonucleotide sequence complementary to vesicular stomatitis virus N protein mRNA initiation site. Proc Natl Acad Sci U S A. 84:648-52.

Letsinger R L, Zhang G R, Sun D K, Ikeuchi T, Sarin P S. 1989. Cholesteryl-conjugated oligonucleotides: synthesis, properties, and activity as inhibitors of replication of human immunodeficiency virus in cell culture. Proc Natl Acad Sci U S A. 86:6553-6.

Liao D. 2000. Arterial stiffness and the development of hypertension. Ann Med. 32:383-5.

Lifton R P, Gharavi A G, Geller D S. 2001. Molecular mechanisms of human hypertension. Cell 104:545-56.

Luft F C. 2001. Mechanisms and cardiovascular damage in hypertension. Hypertension 37:594-598.

Maher L J 3rd. 1992. DNA triple-helix formation: an approach to artificial gene repressors? Bioessays. 14:807-15.

Majid D S, Said K E, Omoro S A, Navar L G. 2001. Nitric oxide dependency of arterial pressure-induced changes in renal interstitial hydrostatic pressure in dogs. Circ Res 88:347-351.

Meade H, Gates L, Lacy E, Lonberg N. 1990. Bovine alpha S1-casein gene sequences direct high level expression of active human urokinase in mouse milk. Biotechnology (New York). 8:443-6.

Moreno C, Maier K G, Hoagland K M, Yu M, Roman R J. 2001. Abnormal pressure-natriuresis in hypertension: role of cytochrome P450 metabolites of arachidonic acid. Am J Hypertens. 14:90S-97S.

Mulvany M J. 2002. Small artery remodelling in hypertension. Curr Hypertens Rep 4:49-55.

Murray C J L, Lopez A D. 1997. Global mortality, disability, and the contribution of risk factors: Global Burden of Disease Study. Lancet 349:1436-42.

Nielsen P E, Egholm M, Berg R H, Buchardt O. 1991. Sequence-selective recognition of DNA by strand displacement with a thymine-substituted polyamide. Science. 254:1497-500.

Oparil S, Zaman M A, Calhoun D A. 2003. Pathogenesis of hypertension. Ann Intern Med. 139:761-76.

Palatini P, Julius S. 1999. Relevance of heart rate as a risk factor in hypertension. Curr Hypertens Rep 3:219-224.

Palmer B F. 2001. Impaired renal autoregulation: implications for the genesis of hypertension and hypertension-induced renal injury. Am J Med Sci 321:388-400.

Peltonen L, Jalanko A, Varilo T. Molecular genetics of the Finnish disease heritage. 1999. Hum Mol Genet. 8:1913-23.

Sajantila A, Salem A H, Savolainen P, Bauer K, Gierig C, Paabo S. 1996. Paternal and maternal DNA lineages reveal a bottleneck in the founding of the Finnish population. Proc Natl Acad Sci U S A. 93:12035-9.

Salonen J T, Lakka T A, Lakka H-M, Valkonen V-P, Everson S A, Kaplan G A. 1998. Hyperinsulinemia is associated with the Incidence of hypertension and dyslipidemia in middle-aged men. Diabetes 47:270-275.

Salonen J T, Malin R, Tuomainen T P, Nyyssonen K, Lakka T A, Lehtimaki T. 1999. Polymorphism in high density lipoprotein paraoxonase gene and risk of acute myocardial infarction in men: prospective nested case-control study. BMJ. 319:487-9.

Salonen J T, Puska P, Kottke T E, Heinonen O P. 1981. Coronary risk factor clustering patterns in Eastern Finland. Int J Epidemiol 10:203-210.

Salonen J T, Salonen R, Seppanen K, Rauramaa R, Tuomilehto J. 1991. High density lipoprotein, HDL2 and HDL3 subfractions and the risk of acute myocardial infarction: a prospective population study in Eastern Finnish men. Circulation 84:129-139.

Salonen J T. 1988. Is there a continuing need for longitudinal epidemiologic research? The Kuopio Ischaemic Heart Disease Risk Factor Study. Ann Clin Res 20: 46-50. Schobel H P, Schmieder R E, Gatzka C D, Messerli F H. 1993. A centripetal shift in intravascular volume triggers the onset of early cardiac adaptation in hypertension. J Hypertens 11:S94-S95.

Segers P, Stergiopulos N, Westerhof N. 2000. Quantification of the contribution of cardiac and arterial remodeling to hyper-tension. Hypertension 36:760-765.

Sevon P, Ollikainen V, Onkamo P, Toivonen H T, Mannila H, Kere J. 2001. Mining associations between genetic markers, phenotypes, and covariates. Genet Epidemiol. 21 Suppl 1:S588-93.

Shimkets R A, Warnock D G, Bositis C M, Nelson-Williams C, Hansson J H, Schambelan M, Gill J R Jr, Ulick S, Milora R V, Findling J W, et al. 1994. Liddle's syndrome: heritable human hypertension caused by mutations in the beta subunit of the epithelial sodium channel. Cell. 79:407-14.

Slotwiner D J, Devereux R B, Schwartz J E, Pickering T G, de Simone G, Roman M J. 2001. Relation of age to left ventricular function and systemic hemodynamics in uncomplicated mild hypertension. Hypertension. 37:1404-9.

Smithies O, Gregg R G, Boggs S S, Koralewski M A, Kucherlapati R S. 1985. Insertion of DNA sequences into the human chromosomal beta-globin locus by homologous recombination. Nature. 317:230-4.

Staessen J A, Wang J, Bianchi G, Birkenhager W H. 2003. Essential hypertension. Lancet. 361:1629-41.

Staessen J A, Wang J G, Brand E, Barlassina C, Birkenhager W H, Herrmann S M, Fagard R, Tizzoni L, Bianchi G. 2001. Effects of three candidate genes on prevalence and incidence of hypertension in a Caucasian population. J Hypertens. 19:1349-58.

Stamler J, Caggiula A W, Gandits G A. 1997. Relation of body mass and alcohol, nutrient, fiber, and caffeine intakes to blood pressure in the special intervention and usual care groups in the Multiple Risk Factor Intervention Trial. Am J Clin Nutr 659(Suppl 1):338S-365S.

Stein C A, Cohen J S. 1988. Oligodeoxynucleotides as inhibitors of gene expression: a review. Cancer Res. 48:2659-68.

Su E J, Lombardi D M, Siegal J, Schwartz S M. 1998. Angiotensin II induces vascular smooth muscle cell replication independent of blood pressure. Hypertension 31:1331-1337.

Syvanen A-C. 2001. Accessing genetic variation: Genotyping single nucleotide polymorphisms. Nature Reviews Genetics. 2:930-942.

Thomas K R, Capecchi M R. 1987. Site-directed mutagenesis by gene targeting in mouse embryo-derived stem cells. Cell. 51:503-12.

Thompson S, Clarke A R, Pow A M, Hooper M L, Melton D W. 1989. Germ line transmission and expression of a corrected HPRT gene produced by gene targeting in embryonic stem cells. Cell. 56:313-21.

Tobian L. Salt and hypertension. 1991. Lessons from animal models that relate to human hypertension. Hypertension 17(Suppl 1):152-158.

Toivonen H T, Onkamo P, Vasko K, Ollikainen V, Sevon P, Mannila H, Herr M, Kere J. 2000. Data mining applied to linkage disequilibrium mapping. Am J Hum Genet. 67:133-45.

Touyz R M. 2000. Molecular and cellular mechanisms regarding vascular function and structure. Can J Cardiol 16:1137-1146.

Touyz R M. 2003. Recent advances in intracellular signalling in hypertension. Curr Opin Nephrol Hypertens. 12:165-74.

Tummala P E, Chen X L, Sundell C L, Laursen J B, Hammes C P, Alexander R W, Harrison D G,

Medford R M. 1999. Angiotensin II induces vascular cell adhesion molecule-1 expression in rat vasculature: A potential link between the renin-angiotensin system and atherosclerosis. Circulation. 100:1223-9.

Tuomainen T P, Kontula K, Nyyssonen K, Lakka T A, Helio T, Salonen J T. 1999. Increased risk of acute myocardial infarction in carriers of the hemochromatosis gene Cys282Tyr mutation: a prospective cohort study in men in eastern Finland. Circulation. 100:1274-9.

Turner S T, Schwartz G L, Chapman A B, Boerwinkle E. 2001. C825T polymorphism of the G protein beta(3)-subunit and antihypertensive response to a thiazide diuretic. Hypertension. 37:739-43.

van der Krol A R, Mol J N, Stuitje A R. 1988. Modulation of eukaryotic gene expression by complementary RNA or DNA sequences. Biotechniques 6:958-76.

van Paassen P, de Zeeuw D, de Jong P E, Navis G. 2000. Renin inhibition improves pressure natriuresis in essential hyper-tension. J Am Soc Nephrol 11:1813-1818.

Weinberger M H. Salt sensitivity of blood pressure in humans. 1996. Hypertension 27:481-490.

Wilcox C S. 2002. Reactive oxygen species: roles in blood pressure and kidney function. Curr Hypertens Rep 4:160-166.

Wilson F H, Disse-Nicodeme S, Choate K A, Ishikawa K, Nelson-Williams C, Desitter I, Gunel M, Milford D V, Lipkin G W, Achard J M, Feely M P, Dussol B, Berland Y, Unwin R J, Mayan H, Simon D B, Farfel Z, Jeunemaitre X, Lifton R P. 2001. Human hypertension caused by mutations in WNK kinases. Science. 293:1107-12.

World Health Organization. Cardiovascular diseases: prevention and control. Information sheet. http://www.who.int/dietphysicalactivity/media/en/gsfs_cvd.pdf (Accessed 14.07.04)

Zon G. 1988. Oligonucleotide analogues as potential chemotherapeutic agents. Pharm Res. 5:539-49.

1. A method for identification of an individual who has an altered risk of or susceptibility for developing HT, the method comprising the steps of:
a) providing a biological sample taken from said individual;
b) collecting personal and clinical information of said individual;
c) determining the nucleotides present in one or several of the polymorphic sites as set forth in tables 2 to 5 and 7 to 11 in said individual's nucleic acid; and
d) combining the SNP marker data with personal and clinical information to assess the risk of an individual to develop HT.

2. The method according to claim 1, wherein the altered risk is an increased risk of HT.

3. The method according to claim 1, wherein the altered risk is a decreased risk of HT.

4. The method according to claim 1, wherein the polymorphic sites are those present in the haplotypes presented in tables 3, 4, 5, 7 and 8.

5. The method according to claim 1, wherein the polymorphic sites are associated with the SNP markers set forth in tables 2 to 5 and 7 to 11.

6. The method according to claim 5, wherein the polymorphic sites are in complete linkage disequilibrium with the SNP markers set forth in tables 2 to 5 and 7 to 11.

7. The method according to claim 6, wherein the polymorphic sites are in complete linkage disequilibrium in the population in which the said method is used.

8. A method for identification of an individual who has an altered risk of or susceptibility for developing HT, the method comprising the steps of
a) providing a biological sample taken from a subject
b) determining the nucleotides present in one or several of the polymorphic sites as set forth in tables 2 to 5 and 7 to 11 in said individual's nucleic acid
c) combining the SNP marker data to assess the risk of an individual to develop HT

9. The method according to claim 8, wherein the altered risk is an increased risk of HT.

10. The method according to claim 8, wherein the altered risk is a decreased risk of HT.

11. The method according to claim 8, wherein the polymorphic sites are those present in the haplotypes presented in tables 3, 4, 5, 7 and 8.

12. The method according to claim 8, wherein the polymorphic sites are associated with the SNP markers set forth in tables 2 to 5 and 7 to 11.

13. The method according to claim 12, wherein the polymorphic sites are in complete linkage disequilibrium with the SNP markers set forth in tables 2 to 5 and 7 to 11.

14. The method according to claim 13, wherein the polymorphic sites are in complete linkage disequilibrium in the population in which the said method is used.

15. The method according to claim 1, wherein said one or several polymorphic sites reside within a HT risk gene or genes as set forth in table 6.

16. The method according to claim 1, wherein the HT risk genes reside in the genome regions which are defined by the haplotype pattern mining analysis, the genes set forth in tables 3, 4, 5, 7 and 8.

17. The method according to claim 1, wherein the polymorphic sites are associated with the haplotype regions, haplotypes or SNP markers defining the haplotypes set forth in tables 3, 4, 5, 7 and 8.

18. The method according to claim 17, wherein the polymorphic sites are in complete linkage disequilibrium with the haplotype regions, haplotypes or SNP markers defining the haplotypes set forth in tables 3, 4, 5, 7 and 8.

19. The method according to claim 18, wherein the polymorphic sites are in complete linkage disequilibrium in the population in which the said method is used.

20. The method according to claim 1, wherein one or several of the SNP markers are selected from the group consisting of the following haplotypes or individual SNPs:
a) rs1521409 (A/G) (SEQ ID NO: 544), rs10511365 (C/T) (SEQ ID NO: 316) and rs10511366 (C/T) (SEQ ID NO: 317) defining the haplotype ACT (or nucleotides from the complementary strand);
b) rs10508771 (A/T) (SEQ ID NO: 286), rs3006608 (C/T) (SEQ ID NO: 854), rs10508773 (C/T) (SEQ ID NO: 287) and rs950132 (C/T) (SEQ ID NO: 1325) defining the haplotype TCCC (or nucleotides from the complementary strand);
c) rs2221511 (A/G) (SEQ ID NO: 733), rs4940595 (G/T) (SEQ ID NO: 986), rs1522723 (C/T) (SEQ ID NO: 548) and rs1395266 (C/T) (SEQ ID NO: 476) defining the haplotype ATCC (or nucleotides from the complementary strand);
d) rs1992906 (A/G) (SEQ ID NO: 655) defining the risk allele G;
e) rs10270360 (A/G) (SEQ ID NO: 10) defining the risk allele G;
f) rs1318392 (A/G) (SEQ ID NO: 438) defining the risk allele G;
g) rs2209672 (A/G) (SEQ ID NO: 730) defining the risk allele A;
h) rs503208 (C/G) (SEQ ID NO: 989) defining the risk allele G

21. The method according to claim 1, wherein one or several of the SNP markers are selected from the group consisting of the following haplotypes or individual SNPs:
a) rs1521409 (A/G) (SEQ ID NO: 544), rs10511365 (C/T) (SEQ ID NO: 316) and rs10511366 (C/T) (SEQ ID NO: 317) defining the haplotype ACT (or nucleotides from the complementary strand);
b) rs2221511 (A/G) (SEQ ID NO: 733), rs4940595 (G/T) (SEQ ID NO: 986), rs1522723 (C/T) (SEQ ID NO: 548) and rs1395266 (C/T) (SEQ ID NO: 476) defining the haplotype ATCC (or nucleotides from the complementary strand);
c) rs1997454 (A/G) (SEQ ID NO: 656) defining the risk allele G;
d) rs10270360 (A/G) (SEQ ID NO: 10) defining the risk allele G;
e) rs1318392 (A/G) (SEQ ID NO: 438) defining the risk allele G;
f) rs2209672 (A/G) (SEQ ID NO: 730) defining the risk allele A;
g) rs503208 (C/G) (SEQ ID NO: 989) defining the risk allele G

22. The method according to claim 1, wherein one or several of the SNP markers are selected from the group consisting of the following haplotypes:
a) rs4845303 (A/T) (SEQ ID NO: 980), rs6428195 (C/G) (SEQ ID NO. 1030) and rs1935659 (A/G) (SEQ ID NO: 637) defining the haplotype ACG (or nucleotides from the complementary strand);
b) rs1997454 (A/G) (SEQ ID NO: 656), rs2139502 (A/G) (SEQ ID NO: 709) and rs1519991 (A/C) (SEQ ID NO: 542) defining the haplotype AGC (or nucleotides from the complementary strand);
c) rs1521409 (A/G) (SEQ ID NO: 544), rs10511365 (C/T) (SEQ ID NO: 316) and rs10511366 (C/T) (SEQ ID NO: 317) defining the haplotype ACT (or nucleotides from the complementary strand);
d) rs7679959 (C/G) (SEQ ID NO: 1178), rs10517338 (C/G) (SEQ ID NO: 381) and rs959297 (A/T) (SEQ ID NO: 1338) defining the haplotype CGA (or nucleotides from the complementary strand);
e) rs2278677 (A/G) (SEQ ID NO: 749), rs3886091 (C/G) (SEQ ID NO: 899), rs1998167 (A/G) (SEQ ID NO: 657), rs1998168 (A/G) (SEQ ID NO: 658) and rs2235280 (A/G) (SEQ ID NO: 740) defining the haplotype GCAGG (or nucleotides from the complementary strand);
f) rs10521062 (A/C) (SEQ ID NO: 404), rs10512296 (A/G) (SEQ ID NO: 331), rs1924001 (C/G) (SEQ ID NO: 633) and rs2417359 (A/G) (SEQ ID NO: 784) defining the haplotype AACG (or nucleotides from the complementary strand);
g) rs10508933 (C/G) (SEQ ID NO: 289), rs10509071 (A/G) (SEQ ID NO: 295) and rs10490967 (A/G) (SEQ ID NO: 94) defining the haplotype GGA (or nucleotides from the complementary strand);
h) rs10508771 (A/T) (SEQ ID NO: 286), rs3006608 (C/T) (SEQ ID NO: 854), rs10508773 (C/T) (SEQ ID NO: 287) and rs950132 (C/T) (SEQ ID NO: 1325) defining the haplotype TCCC (or nucleotides from the complementary strand);
i) rs1386486 (C/T) (SEQ ID NO: 472), rs1386485 (A/C) (SEQ ID NO: 471), rs1386483 (A/G) (SEQ ID NO: 470) and rs7977245 (C/T) (SEQ ID NO: 1212) defining the haplotype CAGT (or nucleotides from the complementary strand);
j) rs276002 (A/G) (SEQ ID NO: 814) and rs274460 (A/G) (SEQ ID NO: 810) defining the haplotype AA (or nucleotides from the complementary strand);
k) rs1245383 (A/G) (SEQ ID NO: 430), rs2133829 (C/T) (SEQ ID NO: 707), rs2173738 (C/T) (SEQ ID NO: 722), rs2050528 (C/T) (SEQ ID NO: 677) and rs202970 (C/T) (SEQ ID NO: 671) defining the haplotype GCTTC (or nucleotides from the complementary strand);
l) rs1395266 (C/T) (SEQ ID NO: 476), rs931850 (A/G) (SEQ ID NO: 1303) and rs1522722 (C/T) (SEQ ID NO: 547) defining the haplotype TAC (or nucleotides from the complementary strand);
m) rs2221511 (A/G) (SEQ ID NO: 733), rs4940595 (G/T) (SEQ ID NO: 986), rs1522723 (C/T) (SEQ ID NO: 548) and rs1395266 (C/T) (SEQ ID NO: 476) defining the haplotype ATCC (or nucleotides from the complementary strand);
n) rs2825555 (A/G) (SEQ ID NO: 819), rs2825583 (C/T) (SEQ ID NO: 820), rs2825601 (A/G) (SEQ ID NO: 821), rs2825610 (G/T) (SEQ ID NO: 822) and rs1489734 (A/G) (SEQ ID NO: 532) defining the haplotype ATGGA (or nucleotides from the complementary strand)

23. A method for assessing susceptibility or predisposition to HT in an individual, the method comprising determining alteration of expression levels of one or several of the genes of table 6 in the individual, wherein a difference in expression is indicative of susceptibility to HT.

24. The method according to claim 23, wherein alteration of expression levels is determined by assessing transcription levels of one or several of the genes of table 6 in the individual.

25. The method according to claim 23, wherein alteration of expression levels is determined by assessing translation of mRNAs encoded by one or several of the genes of table 6 in the individual.

26. A method for assessing susceptibility or predisposition to HT in an individual, the method comprising determining alteration of biological activity of one or several ot the polypeptides encoded by one or several of the genes of table 6 in the individual, wherein a difference in biological activity of one or several of the polypeptides is indicative of susceptibility to HT.

27. The method according to claim 26, wherein alteration of biological activity is determined by assessing structure of one or several ot the polypeptides encoded by one or several of the genes of table 6 in the individual.

28. The method according to claim 26, wherein alteration of biological activity is determined by assessing amount of one or several of the metabolites of a polypeptide or polypeptides encoded by one or several of the genes of table 6 in the individual.

29. The method according to claim 1, wherein the personal and clinical information, i.e. non-genetic information concerns age, gender, behaviour patterns and habits, biochemical measurements, clinical measurements, obesity, the family history of HT, cerebrovascular disease, other cardiovascular disease, hypercholesterolemia, obesity and diabetes, waist-to-hip circumference ratio (cm/cm), socioeconomic status, psychological traits and states, and the medical history of the subject.

30. The method according to claim 29, wherein the behaviour patterns and habits include tobacco smoking, physical activity, dietary intakes of nutrients, alcohol intake and consumption patterns and coffee consumption and quality.

31. The method according to claim 29, wherein the biochemical measurements include determining blood, serum or plasma VLDL, LDL, HDL, total cholesterol, triglycerides, apolipoprotein (a), fibrinogen, ferritin, transferrin receptor, C-reactive protein, glucose or insulin concentration.

32. The method according to claim 29, wherein the non-genetic measurements are those presented in table 8.

33. The method according to claim 29, wherein the non-genetic information contains BMI and history of obesity in the family of the subject.

34. The method according to claim 29 further comprising a step of calculating the risk of HT using a logistic regression equation as follows:
Risk of HT=[1+e^{-(a+Σ(bi*Xi)}]⁻¹, where e is Napier's constant, Xᵢ are variables associated with the risk of HT, bᵢ are coefficients of these variables in the logistic function, and a is the constant term in the logistic function.

35. The method according to claim 34, wherein a and bᵢ are determined in the population in which the method is to be used.

36. The method according to claim 34, wherein Xi are selected among the variables that have been measured in the population in which the method is to be used.

37. The method according to claim 34, wherein Xi are selected among the SNP markers of tables 2 to 5 and 7 to 11, among haplotype regions and haplotypes of tables 3, 4, 5, 7 and 8 and among non-genetic variables of the invention.

38. The method according to claim 34, wherein bᵢ are between the values of −20 and 20 and/or wherein Xᵢ can have values between −99999 and 99999 or are coded as 0 (zero) or 1 (one).

39. The method according to claim 34, wherein i are between the values 0 (none) and 100,000.

40. The method according to claim 1, wherein subject's short term, median term, and/or long term risk of HT is predicted.

41. A method for identifying compounds useful in prevention or treatment of HT comprising determining the effect of a compound on biological networks and/or metabolic pathways related to one or several polypeptides encoded by HT risk genes of table 6 in living cells; wherein a compound altering activity of one or several said biological networks and/or metabolic pathways is considered useful in prevention or treatment of HT.

42. The method according to claim 41 comprising determining the effect of a compound on a biological activity of one or several polypeptides encoded by HT risk genes of table 6 in living cells; wherein a compound altering biological activity of a polypeptide is considered useful in prevention and/or treatment of HT.

43. A method for prevention or treatment of HT comprising administering to a mammalian subject in need of such treatment an effective amount of a compound in a pharmaceutically acceptable carrier enhancing or reducing biological activity of one or several polypeptides encoded by HT risk genes of table 6; and/or enhancing or reducing activity of one or several biological networks and/or metabolic pathways related to said polypeptides.

44. The method according to claim 43 comprising administering to a mammalian subject in need of such treatment an effective amount of a compound in a pharmaceutically acceptable carrier enhancing or reducing expression of one or several HT risk genes of table 6; and/or enhancing or reducing the expression of one or several genes in biological networks and/or metabolic pathways related to polypeptides encoded by said HT risk genes.

45. The method according to claim 43 comprising administering to a mammalian subject in need of such treatment an effective amount of a compound in a pharmaceutically acceptable carrier enhancing or reducing activity of one or several pathophysiological pathways involved in cardiovascular diseases and related to polypeptides encoded by HT risk genes of table 6.

46. The method according to claim 43, said method comprising the steps of:
a) providing a biological sample taken from a subject;
b) determining the nucleotides present in one or several of the polymorphic sites associated with altered expression and/or biological activity and present in HT risk genes of table 6 in said individual's nucleic acid; and
c) combining polymorphic site genotype data to select effective therapy for treating HT in said subject.

47. The method according to claim 43, said method comprising the steps of:
a) providing a biological sample taken from a subject;
b) determining expression of one or several HT risk genes of table 6 and/or determining biological activity of one or several polypeptides encoded by the HT risk genes of table 6 in said individual's sample; and
c) combining the expression and/or biological activity data to select effective therapy for treating HT in said subject.

48. The method according to claim 43, wherein said treatment is gene therapy or gene transfer.

49. The method according to claim 48, wherein said treatment comprises the transfer of one or several HT risk genes of table 6 or variants, fragments or derivatives thereof.

50. The method according to claim 48, wherein said HT risk genes of table 6 or variants, fragments or derivatives thereof are associated with reduced risk of HT.

51. The method according to claim 48, wherein said treatment comprises treating regulatory regions and/or gene containing region of one or more HT risk genes of table 6 or variants, fragments or derivatives thereof in somatic cells of said subject.

52. The method according to claim 48, wherein said treatment comprises treating regulatory regions and/or gene containing region of one or more HT risk genes of table 6 or variants, fragments or derivatives thereof in stem cells.

53. The method according to claim 52, wherein said treatment comprises treating regulatory regions and/or gene containing region of one or more HT risk genes of table 6 or variants, fragments or derivatives thereof in stem cells in tissues affected by cardiovascular diseases.

54. The method according to claim 43, wherein said compound is a recombinant polypeptide encoded by an HT risk gene of table 6 or variant, fragment or derivative thereof.

55. The method according to claim 43, wherein said treatment is based on siRNA hybridising to mRNA and/or to hnRNA of a HT risk gene of table 6.

56. The method according to claim 43, wherein said treatment is based on siRNA hybridising to mRNA and/or to hnRNA of one or several genes in biological networks and/or metabolic pathways related to polypeptides encoded by said HT risk genes of table 6.

57. The method according to claim 43, wherein said method of treating is a dietary treatment or a vaccination.

58. The method according to claim 43 comprising a therapy restoring, at least partially, the observed alterations in biological activity of one or several polypeptides encoded by HT risk genes of table 6 in said subject, when compared with HT free healthy subjects.

59. The method according to claim 43 comprising a therapy restoring, at least partially, the observed alterations in expression of one or several HT risk genes of table 6 in said subject, when compared with HT free healthy subjects.

60. A method for monitoring the effectiveness of treatment of HT in a human subject the method comprising measuring mRNA levels of HT risk genes of table 6, and/or levels of polypeptides encoded by said HT risk genes, and/or biological activity of polypeptides encoded by said HT risk genes in a biological sample taken from said subject; alteration of mRNA levels or polypeptide levels or biological activity of a polypeptide following treatment being indicative of the efficacy of the treatment.

61. A method for predicting the effectiveness of a given therapeutic for HT in a given individual comprising screening for the presence or absence of the HT associated SNP markers, haplotypes or haplotype regions in one or several of the HT risk genes of claim 15.

62. A method for predicting the effectiveness of a given therapeutic for HT in a given individual, the method comprising the steps of:
a) providing a biological sample taken from a subject
b) determining the nucleotides present in one or several of the polymorphic sites as set forth in tables 2 to 5 and 7 to 11 in said individual's nucleic acid; and
c) combining the SNP marker data to predict the effectiveness of a given therapeutic in an individual for HT.

63. A method for diagnosing of a subtype of HT in an individual having HT, the method comprising the steps of:
a) providing a biological sample taken from a subject;
b) determining the nucleotides present in one or several of the SNP markers as set forth in tables 2 to 5 and 7 to 11 in said individual's nucleic acid; and
d) combining the SNP marker data to assess the subtype of HT of an individual.

64. The method according to claim 63, wherein said one or several SNP markers reside within a HT risk gene or genes as set forth in table 6.

65. The method according to claim 63, wherein the HT risk genes reside in the genome region which is defined by the haplotype pattern mining analysis, the genes and regions set forth in tables 3, 4, 5, 7 and 8.

66. The method according to claim 63, wherein the polymorphic sites are associated with the haplotype regions, haplotypes or SNP markers defining the haplotypes set forth in tables 3, 4, 5, 7 and 8.

67. The method according to claim 63, wherein the polymorphic sites are in complete linkage disequilibrium with the haplotype regions, haplotypes or SNP markers defining the haplotypes set forth in 3, 4, 5, 7 and 8.

68. The method according to claim 63, wherein the polymorphic sites are in complete linkage disequilibrium in the population in which the said method is used.

69. The method according to claim 61 further comprising a step of combining non-genetic information with the results obtained.

70. The method according to claim 69, wherein the non-genetic information concerns age, gender, behaviour patterns and habits, biochemical measurements, clinical measurements, obesity, the family history of HT, cerebrovascular disease, other cardiovascular disease, hypercholesterolemia, obesity and diabetes, waist-to-hip circumference ratio (cm/cm), socioeconomic status, psychological traits and states, and the medical history of the subject.

71. The method according to claim 69, wherein the behaviour patterns and habits include tobacco smoking, physical activity, dietary intakes of nutrients, alcohol intake and consumption patterns and coffee consumption and quality.

72. The method according to claim 69, wherein the biochemical measurements include determining blood, serum or plasma VLDL, LDL, HDL or total cholesterol or triglycerides, apolipoprotein (a), fibrinogen, ferritin, transferrin receptor, C-reactive protein, glucose, serum or plasma insulin concentration.

73. The method according to claim 69, wherein the non-genetic measurements are those presented in table 8.

74. The method according to claim 69, wherein the non-genetic information contains the BMI and history of obesity in the family of the subject.

75. A method for measuring HT risk gene product protein expression, production or concentration in a biological sample taken from a subject, wherein said HT risk gene is as defined in table 6, the method comprising the steps of:
a) providing a biological sample taken from a subject to be tested; and
b) detecting the expression, production or concentration of said protein in said sample, wherein altered expression, production or concentration indicates an altered risk of cardiovascular disease in said subject

76. A test kit based on a method according to claim 1 for assessment of an altered risk of or susceptibility for HT in a subject.

77. A test kit for determining the nucleotides present in one or several of the SNP markers as set forth in tables 2 to 5 and 7 to 11 in said individual's nucleic acid for assessment of an altered risk of HT in a subject.

78. A test kit for determining the nucleotides present in one or several of the SNP markers as set forth in tables 2 to 5 and 7 to 11 in said individual's nucleic acid for assessment of an altered risk of HT in a subject, containing:
a) reagents and materials for assessing nucleotides present in one or several SNP markers as set forth in tables 2 to 5 and 7 to 11; and
b) software to interpret the results of the determination.

79. The test kit according to claim 76 further comprising PCR primer set for amplifying nucleic acid fragments containing one or several SNP markers as set forth in tables 2 to 5 and 7 to 11 from the nucleic acids of the subject.

80. The test kit according to claim 76 comprising a capturing nucleic acid probe set specifically binding to one or several SNP markers present in HT associated markers and haplotype regions as set forth in tables 2 to 5 and 7 to 11.

81. The test kit according to claim 76 comprising a microarray or multiwell plate to assess the genotypes.

82. The test kit according to claim 76 comprising a questionnaire for obtaining patient information concerning age, gender, height, weight, waist and hip circumference, skinfold and adipose tissue thicknesses, the proportion of adipose tissue in the body, the family history of diabetes and obesity, the medical history concerning HT.

83. A test kit for detecting the presence of SNP markers in one or several of HT risk genes as set forth in table 6 in a biological sample, wherein said SNP markers are more frequently present in a biological sample of a subject susceptible to HT compared to a sample from a subject not susceptible to HT, the kit comprising:
a) reagents and materials for assessing nucleotides present in SNP markers in one or several of HT risk genes as set forth in table 6; and
b) software to interpret the results of the determination.

84. The test kit of claim 83 further comprising PCR primer set for amplifying nucleic acid fragments containing said SNP markers from HT risk genes as set forth in table 6 from the nucleid acids of the subject.

85. The test kit of claim 83 comprising a capturing nucleic acid probe set specifically binding to one or several SNP markers present in HT risk genes as set forth in table 6.

86. The test kit of claim 83 comprising a microarray or multiwell plate to assess the genotypes.

87. The test kit of claim 83 comprising a questionnaire for obtaining patient information concerning age, gender, height, weight, waist and hip circumference, skinfold and adipose tissue thicknesses, the proportion of adipose tissue in the body, the family history of diabetes and obesity, the medical history concerning HT.

88. A test kit based on a method according to claim 46.

89. The test kit of claim 88 further comprising PCR primer set for amplifying nucleic acid fragments containing said SNP markers from HT risk genes as set forth in tables 2 to 5 and 7 to 11 from the nucleid acids of the subject.

90. The test kit of claim 88 comprising a capturing nucleic acid probe set specifically binding to one or several SNP markers present in HT risk genes as set forth in tables 2 to 5 and 7 to 11.

91. The test kit of claim 88 comprising a microarray or multiwell plate to assess the genotypes.

92. The test kit of claim 88 comprising a questionnaire for obtaining patient information concerning age, gender, height, weight, waist and hip circumference, skinfold and adipose tissue thicknesses, the proportion of adipose tissue in the body, the family history of diabetes and obesity, the medical history concerning HT.

93. The test kit of claim 76, further comprising a marker set to assess the ancestry of an individual.

94. The test kit of claim 93 comprising a SNP marker set to assess the ancestry of an individual.

95. The test kit of claim 93 comprising a microsatellite marker set to assess the ancestry of an individual.

96. The method of claim 1 further comprising a marker set to assess the ancestry of an individual.

97. The method of claim 1 comprising a SNP marker set to assess the ancestry of an individual.

98. The method of claim 1 comprising a microsatellite marker set to assess the ancestry of an individual.

99. The method according to claim 1, wherein one or several of the SNP markers are selected from the group consisting of the following individual SNPs:
a) rs1860933 (AT) (SEQ ID NO:1366) defining the risk allele A
b) rs4236780 (CG) (SEQ ID NO:1367) defining the risk allele C
c) rs2000112 (CT) (SEQ ID NO:660) defining the risk allele C
d) rs931850 (AG) (SEQ ID NO:1303) defining the risk allele A
e) rs2192947 (AG) (SEQ ID NO:728) defining the risk allele G
f) rs9328292 (AG) (SEQ ID NO:1316) defining the risk allele A
g) rs1409367 (CT) (SEQ ID NO:490) defining the risk allele C
h) rs1893814 (CT) (SEQ ID NO:622) defining the risk allele T
i) rs2263356 (CT) (SEQ ID NO:746) defining the risk allele T
j) rs6826647 (CT) (SEQ ID NO:1368) defining the risk allele C
k) rs1913157 (CG) (SEQ ID NO:630) defining the risk allele C

100. The method according to claim 99 further comprising a step of combining information from hypertension drug treatment of the subject to the genetic information of the subject.

101. The method according to claim 1, wherein one or several of the SNP markers are selected from the group consisting of the following individual SNPs:
a) rs6826647 (CT) (SEQ ID NO:1368) defining the risk allele C
b) rs1409367 (CT) (SEQ ID NO:490) defining the risk allele C
c) rs9328292 (AG) (SEQ IS NO:1316) defining the risk allele A
d) rs1395266 (CT) (SEQ ID NO:476) defining the risk allele T
e) rs1893814 (CT) (SEQ ID NO:622) defining the risk allele T
f) rs931850 (AG) (SEQ ID NO:1303) defining the risk allele A
g) rs1860933 (AT) (SEQ ID NO:1366) defining the risk allele A
h) rs1386483 (AG) (SEQ ID NO:470) defining the risk allele A
i) rs4236780 (CG) (SEQ ID NO:1367) defining the risk allele C
j) rs1913157 (CG) (SEQ ID NO:630) defining the risk allele C
k) rs2263356(CT) (SEQ ID NO:746) defining the risk allele T
l) rs2000112 (CT) (SEQ ID NO:660) defining the risk allele C
